(19) United States
(12) Patent Application Publication
(10) Pub. No.: US 2026/0315723 A1
(43) Pub. Date: Oct. 15, 2026

(54) SYSTEMS AND METHODS FOR XENON CAPTURE, CONCENTRATION, ADMINISTRATION, RECAPTURE, REGENERATION, AND REUSE

(71) Applicant: Barrett Jon Larson, Palo Alto, CA (US)

(72) Inventor: Barrett Jon Larson, Palo Alto, CA (US)

(21) Appl. No.: 19/642,625

(22) Filed: Apr. 9, 2026

Related U.S. Application Data
(60) Provisional application No. 63/786,213, filed on Apr. 9, 2025.

(51) Int. Cl. A61M 16/10; A61M 16/00; B01D 8/00; B01D 53/22; B01D 53/26; B01D 53/30

(52) U.S. Cl. CPC A61M 16/105; A61M 16/022; B01D 8/00; B01D 53/22; B01D 53/26; B01D 53/30; A61M 2016/1025; A61M 2202/0291; A61M 2209/088; B01D 2256/18; B01D 2257/80; B01D 2259/4533

(57) ABSTRACT

Systems, devices, modules, and methods are provided for recovering, concentrating, purifying, administering, recapturing, regenerating, certifying, storing, blending, and reusing xenon from xenon-containing gas mixtures including ambient air, conditioned ambient air, recirculated or locally enriched atmospheres, scavenging streams, exhaust streams, and other waste or process streams. In various embodiments, a xenon recovery platform includes xenon-selective separation, optional pretreatment, staged concentration, fraction-cut collection, regeneration or other release processing, and storage or blending. Administration embodiments define controlled breathing zones, including semi-open room-scale and personal wearable breathing microenvironments, without requiring a fully closed recirculating breathing loop, and may use local capture, delayed environmental recapture, dehumidification, portable regeneration, active atmosphere-concentration management, or combinations thereof. Facility, biological-conditioning, article, site-utility, and distributed-service embodiments include xenon recovery modules, inserts, regeneration stations, product-handling systems, xenon-bearing gas vessels, exchange-vessel workflows, and local or off-site regeneration, certification, redistribution, and reuse.

### CROSS-REFERENCE TO RELATED APPLICATIONS

This application claims the benefit of U.S. Provisional Patent Application No. 63/786,213, filed Apr. 9, 2025, the entire disclosures of which are incorporated herein by reference to the fullest extent permitted by law.

### BACKGROUND OF THE INVENTION

### Field of the Invention

The present disclosure relates generally to systems, devices, modules, media, and methods for capturing, concentrating, purifying, storing, blending, administering, conditioning, recapturing, regenerating, certifying, and reusing xenon. More particularly, the disclosure relates to xenon capture from ambient air and other xenon-containing gas mixtures, including conditioned air, recirculated air, localized atmospheres, scavenging streams, ventilation pathways, exhaust streams, exhaled-gas pathways, storage or transport atmospheres, and other process or facility streams; to delivery, administration, or atmospheric-conditioning environments for therapeutic, medical, wellness, veterinary, agricultural, horticultural, livestock, aquaculture, post-harvest, produce-preservation, transport, research, industrial, aerospace, imaging, and scientific applications; and to associated infrastructure including pretreatment, structured contactors, regeneration systems, quality-control systems, storage systems, blending systems, monitoring systems, and logistics systems.

In certain embodiments, the disclosure relates to decentralized or point-of-use xenon infrastructure. In certain embodiments, the disclosure relates to facility-scale xenon recapture and reuse. In certain embodiments, the disclosure relates to semi-open or controlled-environment xenon administration or biological conditioning with delayed local environmental recapture. In certain embodiments, the disclosure relates to renewable-powered, grid-powered, hybrid-powered, or otherwise energy-managed xenon systems.

In certain embodiments, the disclosure relates to personal, wearable, portable, body-worn, user-carried, or garment-integrated xenon delivery, recapture, regeneration, and reuse systems configured to create a localized breathing microenvironment without requiring conventional anesthesia-machine architecture.

### Background

Xenon is a rare atmospheric noble gas present at very low concentration, approximately 0.087 parts per million by volume. Xenon has a wide range of valuable applications, including medical, therapeutic, wellness, scientific, imaging, aerospace, electronics, industrial, and specialty applications. In medicine and related human-use settings, xenon has long been recognized as a uniquely valuable gas because of its anesthetic, organ-protective, neurological, imaging, and other physiological properties. However, the cost, availability, and logistics of xenon have constrained broader adoption across many such applications.

Commercial xenon has traditionally been obtained primarily as a minor byproduct of large cryogenic air-separation processes. Such processes are highly centralized, capital intensive, and energy intensive. Xenon obtained in this manner is typically purified, compressed, distributed, and transported through conventional industrial-gas supply chains. This centralized model contributes to price volatility, limited accessibility, logistical burden, and environmental cost, particularly for users who would benefit from point-of-use or near-point-of-use xenon availability.

Conventional xenon-use workflows also tend to be inefficient in the sense that xenon is frequently lost after use or during use. In anesthesia and related settings, xenon may be lost from breathing circuits, seals, interfaces, scavenging paths, room air, exhaust streams, ventilation systems, or other facility pathways. In other use settings, xenon cost has historically favored tightly sealed, highly conservative delivery paradigms and has discouraged broader development of comfortable or semi-open administration environments, local recapture systems, reusable facility infrastructure, and delayed recapture strategies. There remains a need for systems that reduce xenon loss and that make xenon economically reusable across a broader range of environments.

Capturing xenon from true ambient outdoor air presents additional challenges because xenon partial pressure in ambient air is extremely low. Working capacity under ambient conditions can be constrained by low xenon partial pressure, humidity, carbon dioxide, co-adsorption, regeneration burden, void-volume dilution, and purity-management issues. At the same time, xenon-containing gas mixtures encountered in other regimes—including conditioned air, recirculated air, localized treatment environments, scavenging streams, and higher-concentration waste streams—can provide substantially more favorable capture and recapture conditions. Accordingly, there remains a need for xenon infrastructure that is not limited to a single feed regime and that can operate across multiple xenon-containing gas mixtures using appropriate pretreatment, staged concentration, recapture, and purification strategies.

There also remains a need for xenon administration systems that can improve user comfort, workflow, and accessibility without depending exclusively on tightly sealed masks or closed breathing loops. In many treatment, wellness, dental, transport, recovery, and home-use contexts, it is desirable to define a controlled breathing zone that can feel substantially ambient-pressure and low-resistance to a user while still allowing xenon to be recaptured locally or environmentally. Related needs include monitoring of oxygen, carbon dioxide, xenon concentration, occupancy, and purge status; protection of clinicians, staff, and bystanders; and coordination of delivery hardware with recapture, regeneration, and quality assurance.

There also remains a need for a distinct category of personal, wearable, portable, body-worn, or user-carried xenon devices outside conventional anesthesia-machine architecture. Such devices may be desirable for home use, sleep or relaxation support, recovery, travel, outdoor or expedition activity, social or hospitality settings, retreat settings, and supervised clinical use outside traditional operating-room workflows. Existing xenon paradigms centered on sealed masks, bulky closed breathing circuits, or room-scale installations do not adequately address this need. There remains a need for systems that can create a localized xenon breathing microenvironment around an ambulatory or resting user, recover exhaled xenon before full ambient dilution, and optionally regenerate and reuse recovered xenon through portable, dockable, or distributed body-worn architectures.

There also remains a need for xenon systems applicable to non-human biological targets and biological-conditioning environments. Agricultural, horticultural, veterinary, livestock, aquaculture, hatchery, post-harvest, specimen-preservation, seed-handling, transport, and storage settings may benefit from xenon-conditioned atmospheres or localized xenon exposure, yet the cost of xenon has similarly discouraged development of non-pressurized or near-ambient-pressure systems for greenhouses, growth chambers, animal enclosures, veterinary treatment or transport environments, produce-storage rooms, pallet enclosures, truck trailers, shipboard containers, and related biological settings. There remains a need for architectures that permit xenon delivery, recirculation, recapture, regeneration, and reuse across such settings.

In some human or animal care contexts, xenon delivery may be desired not only for treatment, recovery, or wellness, but also for comfort-centered care in palliative, hospice, veterinary, or other legally authorized end-of-life settings. There remains a need for calm, low-resistance administration architectures that can support such settings, together with safety, monitoring, purge, clinician-or caregiver-access, and recapture capabilities, without limiting the disclosed platform to curative or performance-oriented uses.

There remains a further need for a xenon platform that is robust to changes in materials, geometry, workflow, venue, and business model. Future systems may employ different sorbents, membranes, structured contactors, polishing techniques, regeneration modalities, removable or fixed capture modules, on-site or off-site regeneration, and different patterns of facility integration. An effective platform disclosure should therefore not be limited to any single contactor geometry, media chemistry, regeneration mechanism, or deployment setting. It should support future improvements in material science, including newly developed or AI-discovered xenon-selective materials, while preserving coverage over the process architecture, recapture workflows, environments of use, and ecosystem interfaces that create the underlying commercial value.

The present disclosure addresses these and other needs by providing systems and methods for multi-regime xenon capture, concentration, administration, recapture, regeneration, and reuse.

### BRIEF SUMMARY OF THE INVENTION

The present disclosure provides systems, devices, modules, and methods for capturing, concentrating, purifying, storing, blending, administering, recapturing, regenerating, certifying, and reusing xenon from xenon-containing gas mixtures. The disclosure is expressly multi-regime and, in various embodiments, can operate on or with true ambient air, conditioned ambient air, recirculated or semi-enclosed atmospheres, localized enriched atmospheres, exhaled gas, anesthesia waste gas, scavenging gas, purifier flow, ventilation return flow, exhaust flow, industrial off-gas, and combinations thereof.

In one aspect, the disclosure provides a xenon capture platform comprising an intake or gas-routing subsystem, an optional pretreatment subsystem, one or more xenon-selective separation stages, one or more regeneration stages, and a collection subsystem configured to produce a xenon-enriched gas, a further purified xenon product, or a xenon-containing mixture. The platform may be embodied as a standalone outdoor unit, a modular point-of-use system, a facility-integrated system, a mobile system, or a distributed network of systems. In certain embodiments, power is supplied by renewable sources, grid sources, stored electrical energy, waste heat, or combinations thereof. In certain embodiments, the platform is configured for decentralized use near the point of administration, near the point of waste generation, or at a facility recapture point.

In another aspect, the disclosure provides process-intensified concentration architectures. In certain embodiments, an input gas is subjected to pretreatment configured to reduce or manage moisture, carbon dioxide, particulates, biological contaminants, volatile contaminants, or combinations thereof. The pretreated gas is then contacted with one or more xenon-selective separation stages. In certain embodiments, the system further includes staged concentration, including a primary collector, a secondary concentrator, and an optional polishing stage. In certain embodiments, the system includes reduced dead-volume manifolds, microvolume collection regions, deeper vacuum applied to relatively small volumes, fraction-cut collection, analyzer-guided collection, threshold-eligible capture, or multi-bed and/or continuous or semi-continuous swing operation. In certain embodiments, regeneration may be achieved by thermal, vacuum, pressure-swing, purge, Joule, induction, microwave, radiofrequency, electrothermal, field-assisted, cryogenic, or hybrid methods.

In another aspect, the disclosure provides xenon administration environments configured to improve comfort, usability, safety, and recapture economics. In certain embodiments, a system defines a controlled breathing zone for a user at substantially ambient pressure and low inspiratory resistance, while still enabling local capture and/or delayed local environmental recapture. Such environments may include canopies, pods, recliners, chairs, hoods, booths, bays, bedsides, operatories, cabins, vehicles, rooms, home-use systems, wellness environments, dental environments, clinical environments, and combinations thereof. In certain embodiments, delivery is provided without requiring a fully closed recirculating breathing loop or a tight face seal. In certain embodiments, the system further comprises purge logic, oxygen assurance, carbon dioxide management, exposure mitigation, emergency egress features, and ambient or local monitoring. In certain embodiments, xenon concentration within a controlled breathing zone or xenon-enabled space is modulated under feedback control based at least in part on one or more user states associated with one or more users, including physiological, neurological, electrophysiological, biometric, behavioral, positional, activity-related, or supervisory inputs.

In another aspect, the disclosure provides personal, wearable, portable, body-worn, user-carried, or garment-integrated xenon systems configured to create a localized xenon-enriched breathing microenvironment adjacent to a user's nose and/or mouth without requiring a fully closed recirculating breathing loop or a tight face seal. In certain embodiments, exhaled or leaked xenon is intercepted in a facial, neck, collar, chest, textile, or other personal microenvironment region before full ambient dilution, routed through one or more dehumidification and xenon-selective recovery stages, optionally regenerated in a portable or docked subsystem, and reused, blended, stored, or recirculated.

In another aspect, the disclosure provides facility recapture infrastructure configured to recover xenon from facility or process pathways. In certain embodiments, xenon is captured from scavenging lines, anesthesia exhaust, procedure-room air, purifier recirculation, room return air, return ducts, nonrecirculating exhaust, HVAC pathways, dental pathways, clinic recirculation, mechanical-room systems, or other facility capture paths. In certain embodiments, xenon may be recovered near the source, after partial local dilution, after session completion, or through delayed environmental recapture from a xenon-enabled space. In certain embodiments, the recovered xenon is routed to concentration, regeneration, polishing, quality-control, blending, storage, or redistribution subsystems.

In another aspect, the disclosure provides removable and/or permanently installed capture modules and associated regeneration ecosystems. In certain embodiments, a capture module is configured as a removable cartridge, a replaceable cartridge, a reloadable vessel, a fixed bed, a duct module, a purifier module, a pod module, a scavenging module, a wearable module, or a permanently installed contactor. In certain embodiments, one or more such modules are received by a regeneration station configured to regenerate the module, route xenon-bearing fractions, measure one or more quality attributes, and produce a certified xenon product or xenon-enriched gas. In certain embodiments, the system further includes keyed or controlled interfaces, compatibility management, blending control, source tracking, regeneration history, saturation tracking, service logistics, off-site regeneration loops, or combinations thereof.

In another aspect, the disclosure provides systems and methods configured to produce pure xenon, xenon-enriched gas, certified xenon product, or a non-hypoxic xenon-containing mixture. In certain embodiments, such products are suitable for medical, wellness, research, industrial, imaging, aerospace, electronics, propulsion, lighting, or other applications. In certain embodiments, the product may be blended with oxygen, air, or one or more other gases and stored in cylinders, cartridges, tanks, reservoirs, intermediate vessels, distribution lines, or point-of-use devices. In certain embodiments, one or more interfaces may be physically or logically keyed according to concentration, certification status, intended use, or compatibility class.

In another aspect, the disclosure provides xenon biological-conditioning environments for one or more biological targets. In certain embodiments, xenon is delivered to or recaptured from greenhouse zones, growth chambers, hatcheries, incubators, veterinary holding areas, livestock enclosures, produce-storage rooms, pallet enclosures, refrigerated containers, truck trailers, shipboard containers, or other biological-conditioning environments operated at ambient or near-ambient pressure. In certain embodiments, xenon is used for preservation, storage, transport, cultivation, stress mitigation, recovery, or other biological handling, and recovered xenon is routed to regeneration, storage, blending, certification, or reuse.

In another aspect, the disclosure contemplates xenon-selective capture media and separation stages based on adsorption, absorption, membrane transport, condensation, freezing, dissolution, diffusion, electrothermal or field-assisted release, and hybrids thereof. The disclosure is not limited to any named media class or chemistry and expressly contemplates future media, including future porous solids, structured media, mixed-matrix systems, and AI-discovered or later-developed xenon-selective materials, provided such materials are configured to enrich, recover, or otherwise selectively handle xenon relative to an input stream.

In another aspect, the disclosure contemplates co-processing and shared-infrastructure embodiments in which xenon capture is integrated with air purification, moisture handling, atmospheric water harvesting, carbon-dioxide handling, oxygen generation or oxygen assurance, ventilation systems, environmental control systems, or combinations thereof. In such embodiments, the benefit may derive from thermodynamic integration, shared airflow handling, shared pretreatment, shared heat management, shared quality control, shared service logistics, or combinations thereof.

In another aspect, the disclosure provides distributed control and fleet coordination architectures tied to physical system state. In certain embodiments, rule-based, model-based, machine-learning, or hybrid controllers may manage one or more system states including adsorption state, regeneration state, saturation state, quality state, environmental safety state, inventory state, energy state, or logistics state. Such control may be local, distributed, facility-level, fleet-level, or combinations thereof.

The foregoing summary is non-limiting and is provided only to introduce certain example aspects of the disclosure. Additional features, combinations, embodiments, and advantages are described elsewhere herein.

### BRIEF DESCRIPTION OF THE SEVERAL VIEWS OF THE DRAWINGS

FIG. 1 is a schematic system overview showing a xenon capture platform configured to receive xenon-containing gas streams from multiple feed regimes including ambient air, conditioned ambient air, recirculated or locally enriched atmospheres, and waste or scavenging streams, and further showing pretreatment, xenon-selective separation, regeneration, concentration, storage, blending, administration, and recapture subsystems.

FIG. 2 is a schematic view of a process core showing pretreatment, a xenon-selective separation stage, regeneration, a collection region, a secondary concentrator, and an optional polishing stage.

FIG. 3 is a family sheet showing representative structured contactor geometries including tray, laminate or foil, mesh or screen, monolith or honeycomb, radial-flow cartridge, and rotating wheel or drum forms, with additional moving-contactor and foam or fiber forms contemplated but omitted for clarity.

FIG. 4 is a schematic view showing fraction-cut collection, a smaller collection region, and a pressure-reduction subsystem configured to apply greater pressure reduction to the smaller collection region than to an upstream volume.

FIG. 5 is a schematic view of a wind-powered xenon capture embodiment including a rotor or turbine structure, an airflow path, a xenon-selective separation stage, regeneration, and downstream collection or storage.

FIG. 6 is a schematic cutaway view of a modular non-turbine xenon harvester including a body or shell, a xenon-selective separation stage, regeneration, intermediate collection, and storage.

FIG. 7 is a schematic view of a facility xenon recovery architecture showing multiple facility gas paths feeding a shared regeneration and quality-assurance backbone.

FIG. 8 is a schematic view showing a xenon recovery module in a scavenging or waste-gas path and the same xenon recovery module docked into a regeneration station.

FIG. 9 is an exploded schematic view of a representative xenon recovery insert and associated layered construction, the insert being representative of insert forms usable in a return grille, return duct, room recirculator, air purifier, canopy return, booth return, HVAC return module, ventilation exhaust duct, or related airflow path.

FIG. 10 is a schematic view of a xenon-enabled space recovery system including a local capture path, a delayed local environmental recapture path, a xenon recovery module, and a purge or recovery control subsystem.

FIG. 11 is a schematic view of a semi-open xenon administration environment including a canopy or partial headspace structure, a controlled breathing zone, a delivery subsystem, a local capture subsystem, and a delayed local environmental recapture subsystem.

FIG. 12 is a schematic view of a xenon atmosphere-conditioning appliance including a housing, a gas-moving subsystem, a xenon delivery subsystem, a xenon recovery subsystem, and a controller, with sensing located in, on, or external to the appliance.

FIG. 13 is a schematic view of an air-curtain or headspace xenon administration apparatus including at least one delivery plenum and at least one extraction path.

FIG. 14 is a schematic view of a dental or clinic xenon administration and recovery system including a patient-zone delivery subsystem, a first capture path, and a second delayed local environmental recapture path.

FIG. 15 is a family sheet showing representative xenon recovery module article forms.

FIG. 16 is a schematic view of a regeneration station for xenon capture modules.

FIG. 17 is a schematic view of a xenon product handling system including a source vessel or source manifold, an analyzer, and a compatibility-controlled interface.

FIG. 18 is a schematic view of a xenon-bearing gas vessel including a compatibility-controlled outlet and at least one external indicium.

FIG. 19 is a schematic view of a site xenon utility system including a plurality of xenon-enabled spaces, a central or semi-central subsystem, and one or more source vessels, source manifolds, or exchange-vessel interfaces.

FIG. 20 is a schematic view of a distributed xenon recovery ecosystem including multiple sites, a transport or handoff pathway, and a chain-of-custody subsystem.

FIG. 21 is a schematic view of a continuous or quasi-continuous xenon recovery architecture using multiple modules or a moving contactor.

FIG. 22 is a schematic view of a local reuse workflow in which recovered xenon is assigned to a local reuse class and routed for same-site reuse.

FIG. 23 is a schematic view of a centralized mechanical-room xenon recovery skid.

FIG. 24 is a schematic view of a hybrid deployment including both a wind-powered capture node and a facility recapture node feeding a shared downstream subsystem.

FIG. 25 is a family sheet showing representative personal xenon delivery and capture geometries including nasal interfaces, nostril interfaces, circumoral frames, partial face structures, transparent lower-face shells, collar-like boundaries, gaiter-integrated interfaces, and pendants or chest pods, with scarf-, wrap-, and textile-integrated variants contemplated but omitted for clarity.

FIG. 26 is a schematic view of a distributed body-worn xenon system showing a face-adjacent delivery interface, a local capture subsystem positioned adjacent a neck, collar, or chest region, one or more flexible conduits, and a body-worn or user-carried subsystem containing a source vessel, xenon recovery module, pump, battery, controller, or combinations thereof.

FIG. 27 is a schematic process view of a personal xenon recovery loop showing interception of exhaled gas from a personal breathing microenvironment, dehumidification, xenon-selective recovery, portable or dockable regeneration, a reduced-volume collection region, and optional humidification of delivered gas using recovered moisture.

FIG. 28 is a schematic view of a personal exhalation-receiver embodiment showing passive and user-directed exhalation capture into a chest pod, pendant, collar receiver, or similar local receiver, together with a dock, cradle, or station configured for charging, regeneration, refill, or data exchange.

### DETAILED DESCRIPTION OF THE INVENTION

### Definitions

As used in this disclosure, unless the context clearly indicates otherwise:

“Xenon-containing gas mixture” means any gas, vapor, atmosphere, airflow, or process stream containing xenon in any concentration, including ambient air, conditioned ambient air, recirculated air, room air, headspace air, booth air, pod air, vehicle or cabin air, purifier flow, return-duct air, exhaust air, scavenging gas, exhaled gas, anesthesia waste gas, industrial off-gas, and mixtures derived from any of the foregoing.

“Xenon-selective capture medium” means any material, structure, medium, layer, surface, membrane, sorbent, absorbent, adsorbent, condensative element, phase-change element, or hybrid separation element that, under at least one operating condition, preferentially retains, slows, partitions, enriches, transmits, condenses, freezes, dissolves, or otherwise causes xenon to become relatively concentrated with respect to at least one other gas component.

“Xenon-selective separation stage” means a stage or subsystem configured to generate a xenon-enriched gas, xenon-bearing fraction, xenon-depleted fraction, or more purified xenon product from a xenon-containing gas mixture, including without limitation adsorption stages, absorption stages, membrane stages, condensation stages, freezing stages, diffusion-based stages, electrothermal stages, field-assisted stages, or hybrids thereof.

“Xenon-enriched gas” means a gas stream having a xenon concentration greater than that of the corresponding input stream from which it was derived, regardless of whether such stream is suitable for direct end use.

“Xenon-bearing fraction” means a gas stream or released stream portion containing xenon at a concentration above the xenon concentration of the corresponding input stream or above a selected detection or collection threshold, including forecut, product-cut, tail, recycle, or other released portions unless expressly excluded by context.

“Product fraction” or “product cut” means a selected portion of a gas stream or desorbed stream, defined by time, temperature, pressure, composition, detector output, valve state, or any combination thereof, that is retained, routed, discarded, polished, blended, or stored differently from another portion of the same stream.

“Pretreatment” means any upstream or intermediate conditioning step configured to alter one or more properties of a xenon-containing gas mixture prior to or between xenon-selective stages, including without limitation removal or reduction of water, carbon dioxide, particulates, aerosols, volatile compounds, reactive gases, biological matter, temperature excursions, pressure excursions, or combinations thereof. Non-limiting examples of pretreatment include filtration, cyclonic separation, inertial separation, electrostatic precipitation, coalescing, refrigerative condensation, desiccant adsorption (including solid, liquid, and membrane-based desiccants), pressure-swing drying, vacuum-swing drying, chemical scrubbing, catalytic conversion, guard-bed adsorption, electrochemical dehumidification, acoustic droplet coalescence, thermal conditioning, and combinations thereof. Pretreatment may further include atmospheric water harvesting in which water removed from the feed gas is collected for beneficial reuse as a co-product of the xenon capture process.

“Structured contactor” means a support geometry or process-contact geometry configured to promote gas contact with a xenon-selective capture medium, including without limitation trays, meshes, screens, foils, laminates, monoliths, honeycombs, pleated media, radial-flow modules, wheels, drums, rotating elements, fibers, felts, cloths, foams, beads, pellets, washcoats, coated supports, and combinations thereof. For the avoidance of doubt, a bed of beads, pellets, granules, or particulate media contained within a housing, vessel, cartridge, or other bounded flow region is considered a structured contactor within the meaning of this disclosure.

“Staged concentration” means concentration of xenon through two or more successive or coordinated concentration, enrichment, purification, or polishing steps, including without limitation a primary collector, a secondary concentrator, and an optional polishing stage.

“Collection region” means a vessel, manifold region, intermediate volume, microvolume chamber, micro-bed, or other fluid region configured to receive at least a portion of a xenon-bearing fraction during or after regeneration, fractionation, concentration, or transfer.

“Internal free-gas volume” means the gas-occupiable volume of a housing, vessel, collector, stage, chamber, or other fluid region, excluding the solid volume of media, supports, structural internals, or other non-gas-occupiable material within that region.

“Pressure-reduction subsystem” means any subsystem configured to reduce pressure in a selected volume, including without limitation a vacuum pump, dry pump, ejector, Venturi device, compressor operated to create reduced pressure, pressure-swing apparatus, or combinations thereof.

“Xenon-release processing” means any step or sequence of steps that releases retained xenon from a capture medium, trap, module, or article, including warming, thawing, pressure reduction, purge-assisted release, dissolution, chemical displacement, cryogenic extraction, or other processing, whether or not the medium is immediately restored to capture-ready state. For the avoidance of doubt, xenon-release processing may be regenerative or non-regenerative.

“Regeneration” means processing of a xenon recovery module or xenon-selective capture medium to release retained xenon and, in many embodiments, restore at least part of its subsequent capture capacity for reuse, including without limitation thermal swing, vacuum swing, pressure swing, purge gas, cooling, heating, Joule heating, induction, microwave, radiofrequency, electromagnetic energy, electrothermal energy, field-assisted release, mechanical agitation, or chemical displacement.

“Controlled breathing zone” means a localized gaseous region adjacent to, surrounding, or directed toward a user's nose and/or mouth and configured for intended inhalation, whether or not bounded by a sealed interface.

“Pressure-neutral” means configured such that a user breathes at or near ambient pressure without requiring a high-resistance sealed breathing interface.

“Semi-open administration environment” means an administration setting in which xenon is delivered for user inhalation without requiring a fully closed recirculating breathing loop or a tight face seal, and in which xenon may be recaptured locally, environmentally, or both.

“Delayed local environmental recapture” means recovery of xenon after xenon has entered a localized environment, including without limitation recovery through local exhaust, perimeter capture, canopy return, headspace capture, room recirculation, purifier recirculation, return-duct capture, exhaust capture, floor or low-point return, overhead return, or other defined local pathway.

“Xenon-enabled space” means a room, pod, booth, bay, chair, vehicle, cabin, operatory, treatment space, workstation area, greenhouse zone, growth chamber, incubator, kennel, stall, pen, coop, storage room, pallet enclosure, shipping container, refrigerated container, or other environment configured to administer, condition, preserve, recapture, monitor, or otherwise intentionally handle xenon.

“Biological target” means a human, non-human animal, plant, harvested produce, seed, egg, embryo, tissue, organ, culture, colony, biologic sample, or other biological material for which xenon exposure, xenon-conditioned atmosphere, xenon-associated preservation, calming, recovery, storage, cultivation, transport, or other biological handling is intended.

“Biological-conditioning environment” means an environment configured to expose one or more biological targets to a xenon-containing atmosphere or to recapture xenon from such exposure, including without limitation a greenhouse zone, growth chamber, hatchery, incubator, veterinary holding environment, livestock enclosure, produce-storage room, pallet enclosure, truck trailer, shipboard container, or other storage or transport volume.

“Personal xenon device” means a device or system configured for use by or on a single user and distinct from a conventional anesthesia machine or full-room installation, including wearable, portable, body-worn, user-carried, bedside, dockable, or garment-integrated forms.

“Personal breathing microenvironment” means a localized gaseous region adjacent to a user's nose and/or mouth that is intentionally enriched or otherwise conditioned with xenon and that is shaped or maintained without requiring saturation of an entire room or a fully closed breathing circuit.

“Personal delivery interface” means any interface, structure, subsystem, diffuser, conduit termination, boundary geometry, or combination thereof configured to deliver xenon to a user's personal breathing microenvironment, including without limitation nasal interfaces, nostril interfaces, nasal clips, cannulas, circumoral frames, partial face structures, transparent lower-face shells, collar-like boundaries, gaiter-integrated interfaces, scarf-or wrap-integrated interfaces, textile-integrated interfaces, pendant-adjacent diffusers, headrest-adjacent structures, or combinations thereof.

“Personal microenvironment region” means a localized region adjacent to a user's lower face, neck, collar, chest, shoulder, garment, accessory, or another body-adjacent structure through which exhaled or leaked xenon may be intercepted before full ambient dilution.

“Wearable or body-worn subsystem” means any subsystem configured to be worn on the head, face, neck, torso, waist, shoulder, limb, or garment of a user, or integrated into apparel, jewelry, accessories, load-bearing gear, or combinations thereof.

“Distributed body-worn architecture” means an arrangement in which a first xenon-related subsystem is positioned adjacent a user's face and a second xenon-related subsystem is positioned elsewhere on the user's body or carried by the user.

“Exhalation receiver” means a structure positioned to receive natural exhalation or user-directed exhalation from a user for later capture, conditioning, xenon recovery, or combinations thereof.

“Respiration-phase state” means an actual, inferred, or predicted inhalation state, exhalation state, transition state, pause state, or respiratory pattern state.

“Facility capture path” means any purposeful pathway within a building, vehicle, treatment environment, or process environment through which xenon-containing gas may be routed, contained, measured, delayed, recaptured, regenerated, or discharged, including without limitation scavenging lines, exhaust lines, return ducts, room recirculators, air purifiers, mechanical-room systems, and nonrecirculating exhaust pathways. As used herein, “facility gas path” refers to a gas-bearing portion of a facility capture path. In many embodiments, “facility gas path” and “facility capture path” refer to the same routed xenon-containing pathway unless the context indicates otherwise.

“Local capture subsystem” means any subsystem configured to capture xenon adjacent to or within a controlled breathing zone during administration.

“Delayed local environmental recapture subsystem” means any subsystem configured to perform delayed local environmental recapture as defined herein.

“Xenon recovery module” means any contactor, cartridge, cassette, bed, monolith, purifier-integrated element, duct-integrated element, scavenging element, recirculation element, wearable element, or other apparatus configured to increase xenon concentration in, or to isolate xenon from, a xenon-containing gas mixture, and capable of regeneration, refreshing, replacement, servicing, or combinations thereof for subsequent use.

As used herein, “xenon recovery module” is the broader term encompassing removable, replaceable, reloadable, serviceable, and permanently installed forms configured to recover xenon from a xenon-containing gas mixture.

“Capture module” means a xenon recovery module configured primarily for capture, concentration, or pretreatment of xenon-containing gas, as distinguished in some embodiments from modules used primarily for polishing, blending, storage transfer, or downstream product handling.

“Xenon recovery insert” means an article configured for placement in an airflow path of a return grille, return duct, room recirculator, air purifier, canopy return, booth return, HVAC return module, ventilation exhaust duct, or another recirculation or exhaust path, and carrying or associated with one or more xenon-selective media.

“Regeneration station” means any device or system configured to receive, connect to, regenerate, service, analyze, certify, refill, reload, or otherwise process one or more xenon recovery modules, capture modules, or xenon-containing cartridges or vessels.

“Service station” means any station, subsystem, or apparatus configured to inspect, service, refresh, replace, verify, or otherwise handle a xenon recovery module, xenon recovery insert, or related article without necessarily regenerating xenon therefrom.

“Extraction station” means any station, subsystem, or apparatus configured to recover xenon from a loaded xenon recovery module, xenon recovery insert, or related article by thermal, vacuum, cryogenic, chemical, dissolution, or hybrid processing.

“Capture node” means a standalone unit, module, subsystem, appliance, facility-integrated subsystem, or other localized element configured to perform at least one of xenon capture, concentration, recapture, regeneration, storage, blending, or quality assurance and to share one or more downstream subsystems with one or more other nodes.

“Eligibility-gated capture” means capture, regeneration, concentration, collection, polishing, blending, storage, or release initiated or modified only when one or more conditions are satisfied, including without limitation xenon concentration threshold, saturation threshold, power availability, safety status, environmental conditions, demand forecast, or quality-control criteria.

“Xenon state” means a measured, inferred, or predicted xenon concentration, xenon inventory, xenon flow condition, xenon distribution condition, xenon delivery condition, xenon capture condition, or combination thereof associated with a gas stream, vessel, module, breathing microenvironment, xenon-enabled space, or user-adjacent region.

“Xenon-state estimator” means any model-based, sensor-fusion-based, rule-based, statistical, computational, or hybrid subsystem configured to estimate xenon concentration, xenon inventory, xenon flow state, or xenon distribution state in a gas stream, vessel, module, xenon-enabled space, or portion thereof based on one or more measured variables including without limitation delivery flow, recovery flow, pressure, temperature, timing, source depletion, ventilation state, occupancy state, or combinations thereof. For the avoidance of doubt, a xenon-state estimator may be implemented as software executed by a controller, as dedicated circuitry, as a distributed sensor-fusion subsystem, or as a combination thereof, and need not directly measure xenon if configured to infer xenon state from one or more correlated measured variables.

“Oxygen state” means a measured, inferred, or predicted oxygen concentration, oxygen sufficiency condition, oxygen delivery condition, user oxygenation condition, or combination thereof associated with a delivered gas, breathing microenvironment, surrounding space, or user.

“User state” means a measured, inferred, predicted, selected, prescribed, or targeted physiological, neurological, electrophysiological, biometric, behavioral, positional, activity-related, supervisory, authorization, or contextual state of a user, including without limitation sleep state, relaxation state, exertion state, motion state, activity state, presence state, oxygen saturation state, respiratory state, heart-rate state, heart-rate-variability state, blood-pressure state, perfusion state, electroencephalography (EEG) state, electroencephalography-derived state, arousal state, sedation state, level-of-consciousness state, clinician-specified target state, supervisory mode, or combinations thereof.

“Controller” means any electronic, electrical, electromechanical, pneumatic, hydraulic, mechanical, analog, digital, software-mediated, rule-based, model-based, or hybrid subsystem configured to manage operation of one or more system elements, including by sensing, determining, actuating, interlocking, scheduling, or otherwise controlling system behavior.

“Quality attribute” means a measured, inferred, verified, or recorded property of a xenon-bearing fraction, xenon-containing gas, xenon recovery module, vessel, batch, or product, including without limitation xenon concentration, oxygen concentration, moisture content, impurity content, pressure, source class, regeneration history, intended use class, certification state, leak status, or combinations thereof.

“Certified xenon product” means any xenon-containing gas, mixture, fraction, storage state, or delivery state for which at least one quality attribute has been measured, inferred, verified, or recorded, including without limitation xenon concentration, oxygen concentration, impurity profile, moisture content, pressure, source history, or regeneration history.

“Non-hypoxic xenon-containing mixture” or “non-hypoxic xenon mixture” means a breathable xenon-containing gas mixture configured to maintain oxygen at or above a selected threshold.

“Source class” means a classification associated with an origin of a xenon-containing gas mixture, xenon-bearing fraction, or loaded module, including without limitation ambient air, conditioned ambient air, recirculated air, locally enriched atmosphere, purifier flow, return-air flow, scavenging gas, anesthesia waste gas, exhaled gas, exhaust gas, industrial off-gas, or combinations thereof.

“Certification state” means a condition or classification indicating whether a xenon-bearing fraction, product, module, vessel, or batch has satisfied one or more selected quality-assurance, release, compatibility, or intended-use criteria.

“Service state” means a state corresponding to an operational, maintenance, authorization, compatibility, payment, inspection, calibration, or other service-related condition of a module, vessel, interface, appliance, or system.

“Identity or state element” means any physical, optical, magnetic, electrical, electronic, logical, or hybrid identifier associated with a module, cartridge, vessel, or batch and configured to indicate, encode, store, or communicate at least one operational, historical, compatibility, or certification state.

“Compatibility-controlled interface” means any mechanical, dimensional, magnetic, optical, electrical, electronic, logical, software-mediated, fluidic, or hybrid interface configured to permit, restrict, or condition coupling, filling, regeneration, blending, storage, dispensing, or release based on one or more compatibility criteria.

“Intended use class” means a class associated with a xenon-bearing fraction, product, module, vessel, or interface and corresponding to a designated downstream use, including without limitation medical use, human-use therapeutic use, wellness use, research use, industrial use, imaging use, aerospace use, or another designated use.

“Compatibility class” means a class associated with a module, vessel, interface, product, or workflow and used to determine whether two or more system elements may be coupled, processed, blended, stored, dispensed, regenerated, or otherwise used together.

“Chain-of-custody state” means a state associated with a module, vessel, or xenon-bearing batch that identifies at least one stage in a workflow selected from capture, loading, storage, transport, regeneration, analysis, certification, blending, dispensing, reuse, or retirement.

“Chain-of-custody subsystem” means any physical, electronic, optical, mechanical, logical, software-mediated, or hybrid subsystem configured to indicate, store, update, verify, transfer, or otherwise manage one or more chain-of-custody states of a module, vessel, xenon-bearing batch, source vessel, exchange-vessel workflow, or combinations thereof.

“Local reuse class” means a class of xenon-bearing gas, xenon-containing mixture, vessel, batch, or module corresponding to suitability for same-site or same-environment reuse when one or more selected concentration, oxygen, impurity, or certification criteria are met, without requiring the highest available purity class.

“Higher-purity class” means a class of xenon-bearing gas, xenon-containing mixture, vessel, batch, or module corresponding to a higher purity, stricter impurity specification, or broader downstream suitability than a local reuse class.

“Source vessel” means a vessel, cylinder, cartridge, tank, buffer vessel, or other container configured to hold xenon-bearing gas, a certified xenon product, or a non-hypoxic xenon-containing mixture for later blending, distribution, administration, or reuse.

“Source manifold” means a conduit system, manifold block, distribution header, or other fluid-distribution arrangement configured to receive, route, blend, distribute, or otherwise convey xenon-bearing gas, a certified xenon product, or a non-hypoxic xenon-containing mixture to one or more vessels, interfaces, or xenon-enabled spaces.

“Exchange-vessel interface” means an interface configured to receive, connect, disconnect, replace, swap, or otherwise manage a source vessel, xenon-bearing gas vessel, recovery vessel, or other vessel within a local or distributed xenon utility workflow.

“Xenon-bearing gas vessel” means any pressure vessel, cylinder, bottle, cartridge, tank, reservoir, buffer vessel, intermediate vessel, or other container configured to contain a xenon-bearing gas or xenon-containing mixture for storage, transport, blending, dispensing, reuse, or combinations thereof.

“External indicium” means any visible, externally accessible, or externally detectable mark, pattern, shape, key, label, band, code, tag, or other outwardly presented feature corresponding to a class, state, compatibility condition, intended use, or certification condition.

“Gas mover” means any device, subsystem, or arrangement configured to cause gas to flow through a process path, including without limitation fans, blowers, compressors, ejectors, venturi devices, pumps, wind-driven structures, natural-draft chimneys, turbine-driven ducts, bellows, diaphragm pumps, peristaltic mechanisms, pressure differentials, or combinations thereof. A gas mover may be active (electrically, mechanically, or pneumatically powered) or passive (wind-driven, buoyancy-driven, or pressure-differential-driven).

“Routing subsystem” means any arrangement of conduits, valves, manifolds, distribution blocks, rotary valves, diverters, check valves, directional flow elements, or other flow-directing structures configured to selectively direct gas to one or more of a collection region, downstream concentrator, storage vessel, polishing stage, analyzer, vent, recycle path, or other destination. A routing subsystem may be actively controlled (by solenoid valves, pneumatic actuators, or electronic controllers) or passively directed (by flow geometry, pressure differential, or gravitational effects).

“Capture interval” means a period of time during which a xenon-containing gas mixture is in contact with a xenon-selective separation stage under conditions promoting selective xenon retention, enrichment, or concentration. A capture interval may be continuous or intermittent, may last from less than one minute to multiple days, and may be initiated, terminated, or modulated by a controller, by environmental conditions, by saturation state, or by demand.

“Xenon-release condition” means a state or set of conditions applied to a xenon-selective separation stage, xenon-selective capture medium, or xenon recovery module that promotes the release, desorption, evaporation, sublimation, displacement, or extraction of retained xenon, including without limitation elevated temperature (at least approximately 50 degrees Celsius above the adsorption temperature), reduced absolute pressure (below approximately 0.5 atmospheres), purge gas flow, electrical energy input, electromagnetic field application, mechanical agitation, chemical displacement, cryogenic extraction, or any combination thereof.

“Primary xenon collector” or “Primary collector” means a first-stage collection or enrichment element within a staged concentration cascade, configured to receive a xenon-containing gas mixture and produce a xenon-enriched intermediate, whether in the form of a loaded sorbent, a gas fraction, or a pressurized or vacuum-released stream. A primary collector is typically larger, higher-throughput, and lower-purity than a downstream secondary concentrator or polishing stage.

“Secondary concentrator” means a second-stage or downstream concentration element within a staged concentration cascade, configured to receive a xenon-enriched intermediate from a primary collector and produce a further enriched or further purified xenon-bearing fraction. A secondary concentrator may be physically smaller and configured for higher purity or higher concentration than the primary collector.

Unless expressly stated otherwise, terms such as “comprising,” “including,” “having,” and similar terms are non-exclusive and permit additional, unrecited elements, stages, or features. Terms such as “about,” “substantially,” and “approximately” encompass normal engineering tolerances, measurement uncertainty, and functionally equivalent variation. Any embodiment described in connection with one subsystem, geometry, material class, or setting may be combined with any other embodiment unless the context expressly indicates otherwise.

### Figure Walkthroughs and Reference Numerals

The drawings are schematic and not necessarily to scale. Like reference numerals generally identify like or analogous elements throughout the figures, although a given numeral need not appear in every figure and a particular figure may omit elements shown elsewhere for clarity. The figures illustrate representative embodiments and operational relationships and are not intended to limit the disclosure to any particular geometry, sequence, material, installation arrangement, or control mode unless expressly stated.

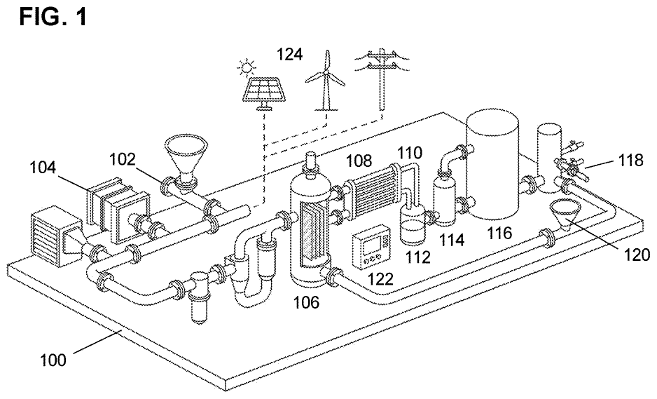

Referring now to FIG. 1, a representative xenon platform 100 includes an inlet flow path 102 configured to receive one or more xenon-containing gas mixtures from one or more feed regimes, an optional pretreatment stage 104, at least one xenon-selective separation stage 106, a regeneration subsystem 108, a collection subsystem 110, an optional secondary concentrator 112, an optional polishing stage 114, one or more storage or source-vessel elements 116, one or more blending or administration subsystems 118, one or more recapture subsystems 120, and a controller 122. In certain embodiments, the platform 100 is powered by one or more power subsystems 124 selected from solar, wind, stored electrical energy, grid power, waste heat, recovered process energy, or combinations thereof.

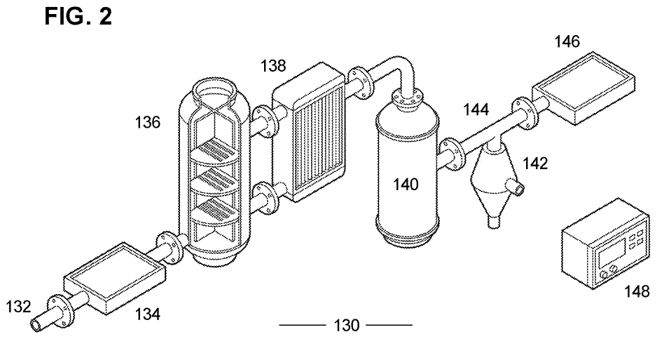

Referring now to FIG. 2, a representative process core 130 receives a xenon-containing gas mixture through an inlet path 132 and routes the mixture through a pretreatment stage 134 and a xenon-selective separation stage 136. In a representative release mode, xenon-bearing fraction is routed through a regeneration path 138 to a collection region 140, then optionally to a secondary concentrator 142, an optional polishing stage 144, and one or more storage, blending, or output destinations 146 under supervision of controller 148.

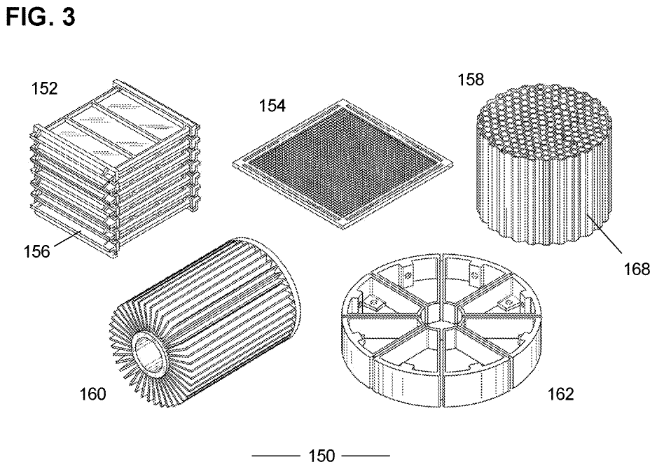

Referring now to FIG. 3, representative structured contactors 150 may be embodied, by way of example, as trays 152, meshes or screens 154, laminates or foils 156, monoliths or honeycombs 158, radial-flow cartridges 160, and rotating wheels or drums 162, as shown. Other contemplated structured contactor forms include moving contactors and fibrous, felt, cloth, foam, bead, pellet, or particulate structures, which may be omitted from the figure for clarity. In certain embodiments, one or more such contactors support a xenon-selective capture medium 168 and are configured for low pressure drop, high mass transfer, improved thermal conduction, improved electrical heating, or combinations thereof.

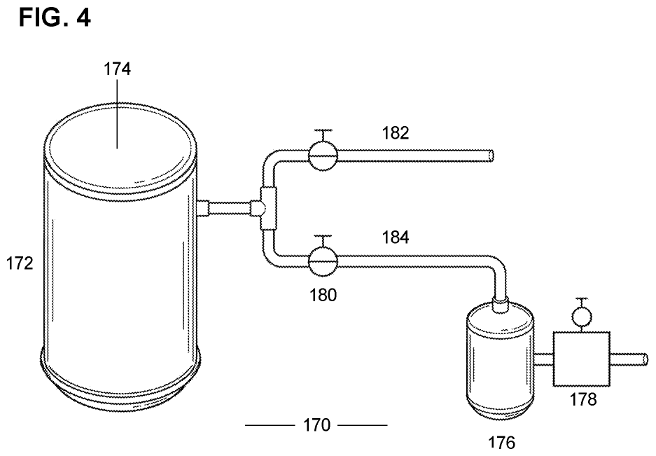

Referring now to FIG. 4, a representative fraction-cut and staged concentration architecture 170 includes a xenon-selective separation stage 172, a first larger internal free-gas volume 174, a smaller collection region 176, a pressure-reduction subsystem 178, and one or more routing valves 180. In certain embodiments, a first non-product fraction 182 is removed, vented, recycled, or otherwise separately handled before a later xenon-bearing fraction 184 is collected in the smaller collection region 176, thereby reducing dead-volume dilution and supporting higher downstream xenon concentration.

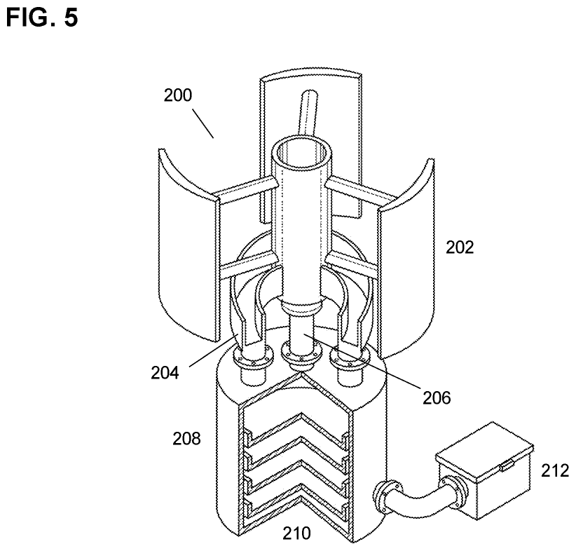

Referring now to FIG. 5, a representative wind-powered xenon capture embodiment 200 includes a rotor or turbine structure 202, one or more blade-adjacent or blade-integrated airflow paths 204, a central collection region 206, at least one xenon-selective separation stage 208, at least one regeneration subsystem 210, and one or more downstream collection, storage, blending, or output subsystems 212. In certain embodiments, the rotor or turbine structure 202 also supplies mechanical power, electrical power, or both to one or more other portions of the xenon capture platform.

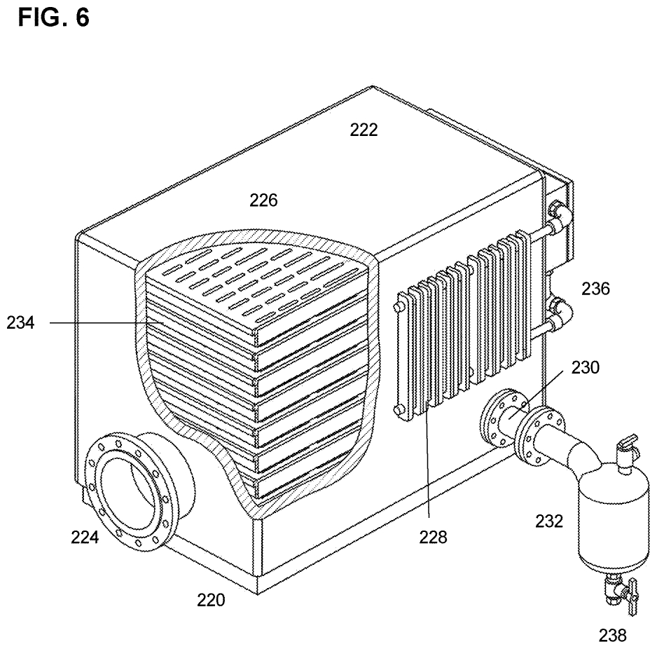

Referring now to FIG. 6, a representative modular non-turbine xenon harvester 220 includes a body or shell 222, an intake or gas-routing subsystem 224, a xenon-selective separation stage 226 disposed within or fluidly coupled to the body or shell 222, a regeneration subsystem 228, an intermediate collection subsystem 230, and an optional compression or storage subsystem 232. In certain embodiments, the xenon-selective separation stage 226 comprises tray-supported or otherwise structured xenon-selective media 234, and regeneration is assisted by one or more heater elements 236 and a vacuum or pressure-reduction path 238.

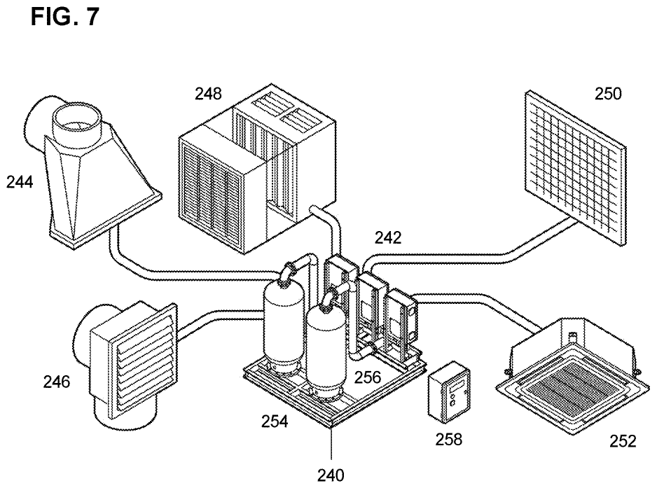

Referring now to FIG. 7, a representative facility xenon recovery architecture 240 includes a plurality of facility capture paths 242, which may include one or more local source-adjacent capture paths 244, delayed local environmental recapture paths 246, room recirculators or purifiers 248, return grilles or return ducts 250, nonrecirculating exhaust paths 252, or mechanical-room skids 254. In certain embodiments, xenon-bearing gas recovered from the plurality of facility capture paths 242 is routed to a shared regeneration and quality-assurance backbone 256 under control of a controller or ventilation interlock subsystem 258.

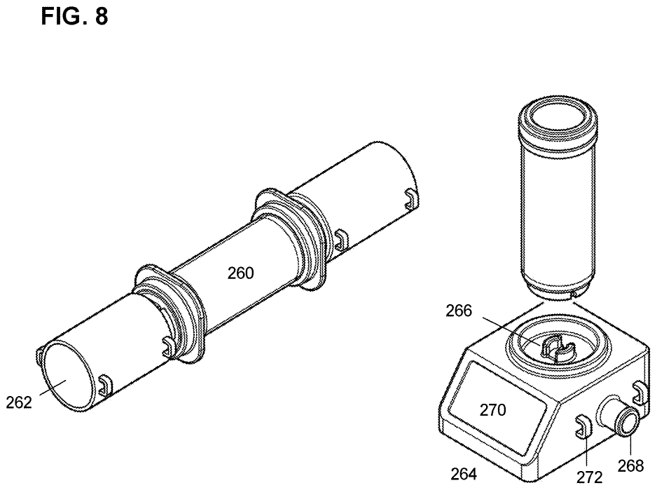

Referring now to FIG. 8, a representative xenon recovery module 260 is positioned in a scavenging, waste-gas, or other capture path 262 and is configured to receive xenon-bearing gas during a capture interval. The xenon recovery module 260 may then be removed and docked into a regeneration station 264 having a docking interface 266, a gas-routing subsystem 268, an analyzer or data subsystem 270, and a release-control subsystem 272, such that gas released from the xenon recovery module 260 may be routed to storage, blending, certification, discard, or other destinations.

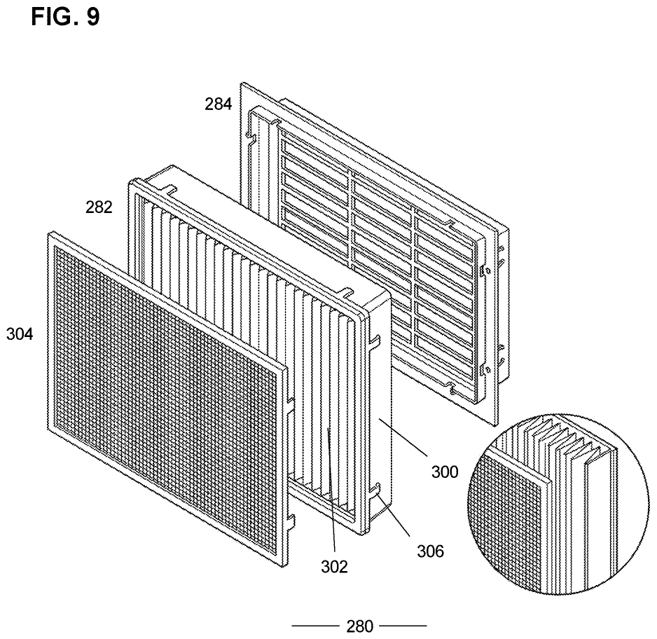

Referring now to FIG. 9, a representative xenon recovery insert 280 includes an insert 282 received within or associated with a return grille or similar airflow structure 284 and representative of insert forms usable in a return duct, room recirculator, air purifier, canopy return, booth return, HVAC return module, ventilation exhaust duct, or combinations thereof. The insert 282 may include a frame or housing 300, a xenon-selective capture medium 302, an optional pretreatment region 304, and one or more service, docking, replacement, or interface features 306.

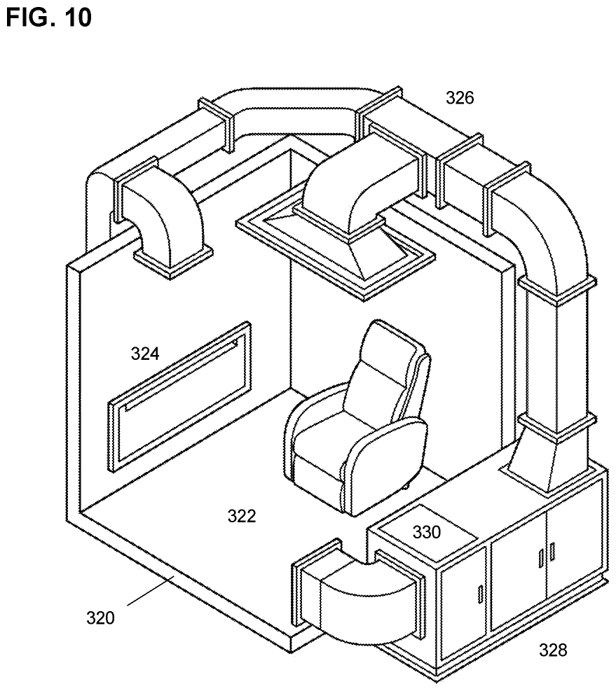

Referring now to FIG. 10, a representative xenon-enabled space recovery system 320 includes a xenon-enabled space 322 associated with a xenon administration session, a local capture path 324, a delayed local environmental recapture path 326, a xenon recovery module 328 positioned in at least one recirculation or recovery path, and a purge or recovery control subsystem 330. In certain embodiments, the system performs recovery during administration, after administration, in a session-end purge mode, or in response to occupancy, concentration, or safety conditions.

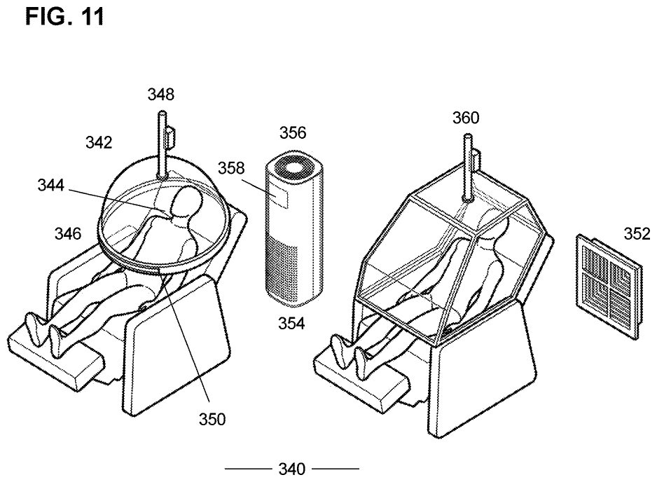

Referring now to FIG. 11, a representative semi-open xenon administration environment 340 includes a canopy or partial headspace structure 342, a controlled breathing zone 344 adjacent to a user 346, a delivery subsystem 348, a local capture subsystem 350, a delayed local environmental recapture subsystem 352, and a safety subsystem 354. In certain embodiments, the delayed local environmental recapture subsystem 352 is external to the canopy or partial headspace structure and is represented schematically by a room-level return or recapture element positioned adjacent the administration environment. In certain embodiments, the safety subsystem 354 cooperates with one or more oxygen sensors 356, xenon sensors or state estimators 358, and a purge path 360 to maintain pressure-neutral or low-resistance breathing while reducing xenon loss to the surrounding environment.

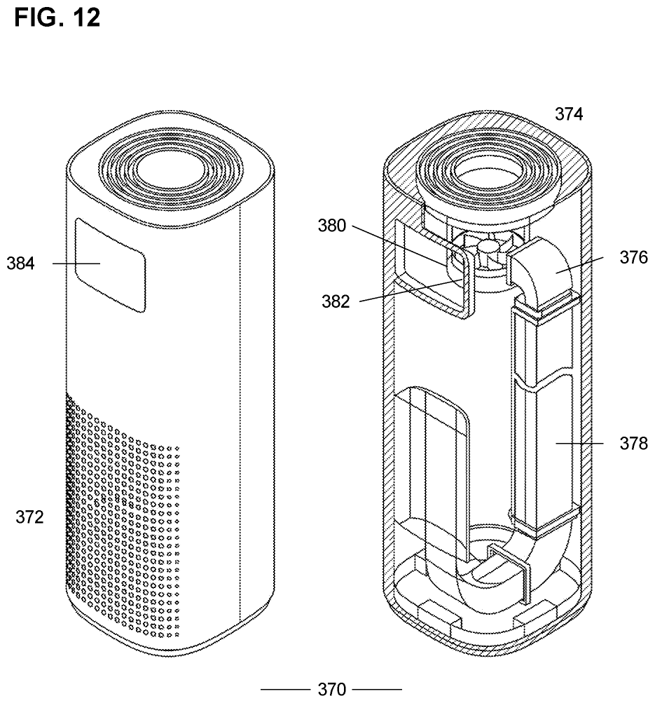

Referring now to FIG. 12, a representative xenon atmosphere-conditioning appliance 370 includes a housing 372, a gas-moving subsystem 374 configured to circulate air from a xenon-enabled space, a xenon delivery subsystem 376, a xenon recovery subsystem 378, an oxygen sensor 380, a xenon sensor or xenon-state estimator 382, and a controller 384. In certain embodiments, the oxygen sensor 380 and the xenon sensor or xenon-state estimator 382 are positioned within, on, or external to the appliance.

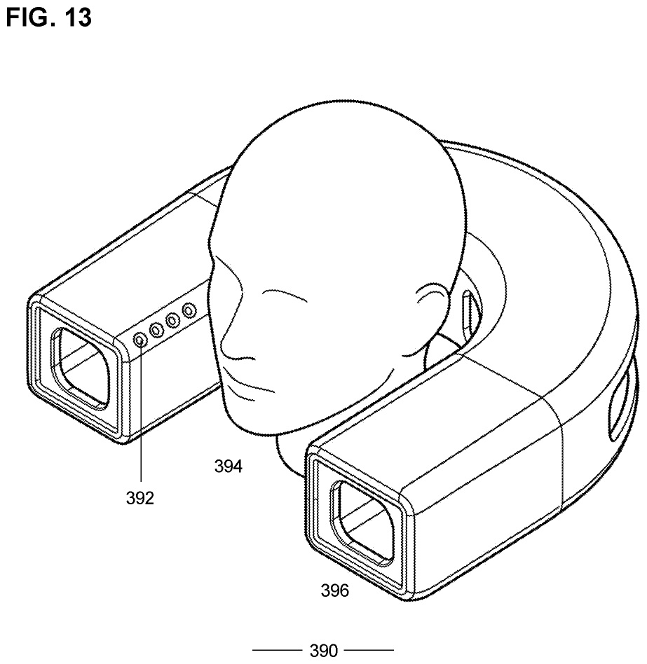

Referring now to FIG. 13, a representative air-curtain or headspace xenon administration apparatus 390 includes at least one delivery plenum, nozzle, diffuser, or manifold 392 configured to form a controlled breathing zone 394 around a user's face and at least one extraction plenum, slot, ring, or return path 396 positioned to capture at least part of the shaped flow field after passage through the controlled breathing zone 394. A xenon recovery subsystem or delayed local environmental recapture path may be fluidly coupled downstream and may be omitted from the figure for clarity.

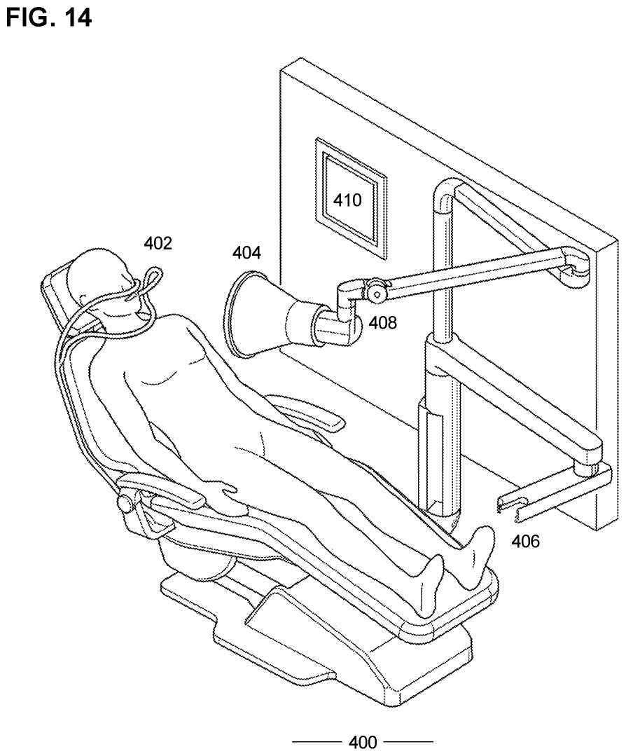

Referring now to FIG. 14, a representative dental or clinic xenon administration and recovery system 400 includes a patient-zone delivery subsystem 402 configured to deliver a xenon-containing gas mixture while leaving an oral treatment field accessible to a clinician, a first capture path 404 positioned adjacent to the patient zone, and a second delayed local environmental recapture path 406 positioned in an operatory or room. In certain embodiments, the system further includes a staff-zone barrier or suction subsystem 408 and one or more monitoring or purge subsystems 410.

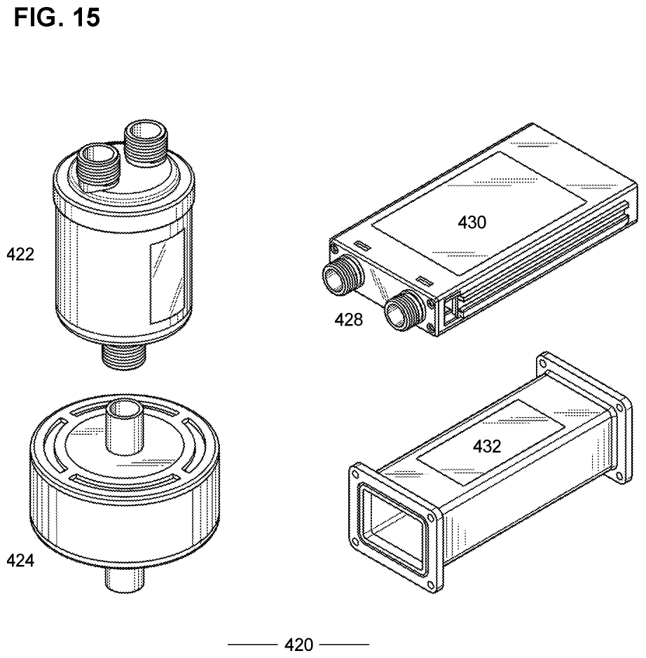

Referring now to FIG. 15, a representative xenon recovery module article family 420 includes representative module forms 422. Representative family features shown across the illustrated forms include a housing 424, an interface 428, and optionally an identity or state element 430 or an external indicium 432 corresponding to source class, service state, compatibility class, saturation state, certification state, or combinations thereof. One or more xenon-selective capture media may be disposed internally and omitted from the figure for clarity.

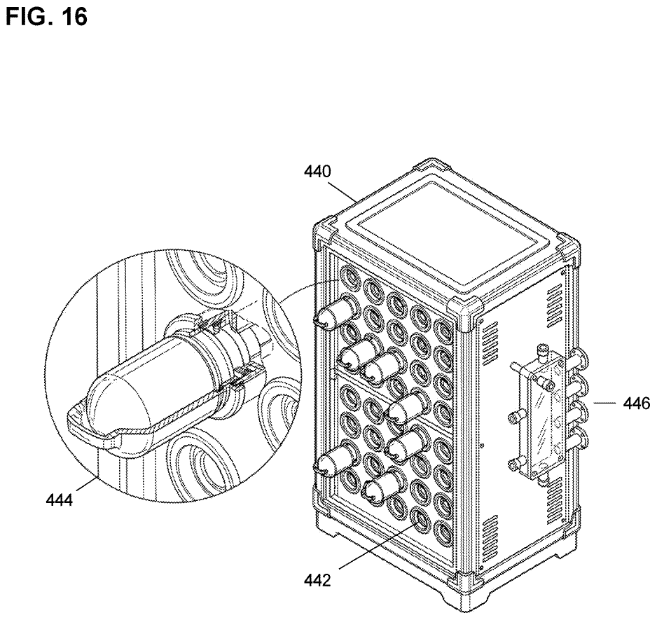

Referring now to FIG. 16, a representative regeneration station 440 includes a docking interface 442 configured to receive one or more xenon recovery modules, a regeneration subsystem 444, a gas-routing subsystem 446, and one or more analyzer, data, or release-control subsystems integrated within or coupled to the station. In certain embodiments, the regeneration station 440 is configured to separate early and later fractions, route recovered gas to storage, blending, certification, discard, or reuse, and update a state of a docked module based on leak testing, regeneration, analysis, or service operations.

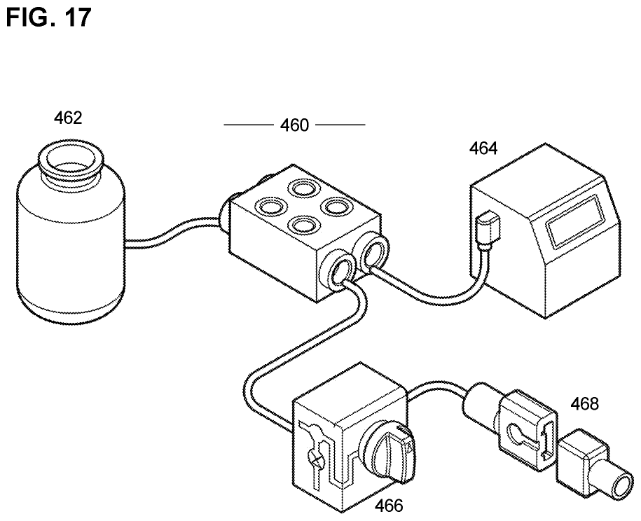

Referring now to FIG. 17, a representative xenon product handling system 460 includes a source vessel or source manifold 462, an analyzer or data subsystem 464 configured to determine or access one or more quality attributes, a release-control subsystem 466, and a compatibility-controlled interface 468. In certain embodiments, the xenon product handling system 460 classifies recovered xenon-bearing gas according to concentration class, intended-use class, local reuse class, higher-purity class, certification state, service state, or combinations thereof and controls filling, storage transfer, blending, or dispensing accordingly.

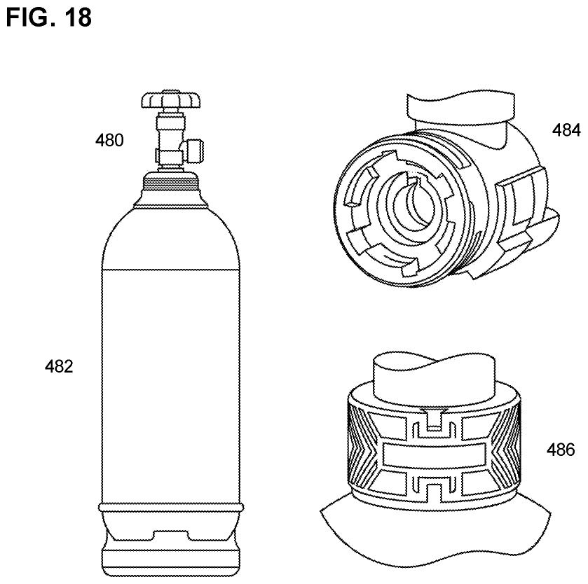

Referring now to FIG. 18, a representative xenon-bearing gas vessel 480 includes a pressure vessel 482, an outlet interface 484 having a compatibility-controlled geometry or other compatibility-controlled feature, and at least one external indicium 486 corresponding to a concentration class, intended-use class, certification state, local reuse class, higher-purity class, service state, or combinations thereof. In certain embodiments, the at least one external indicium 486 cooperates with the outlet interface 484 to differentiate permitted from non-permitted coupling.

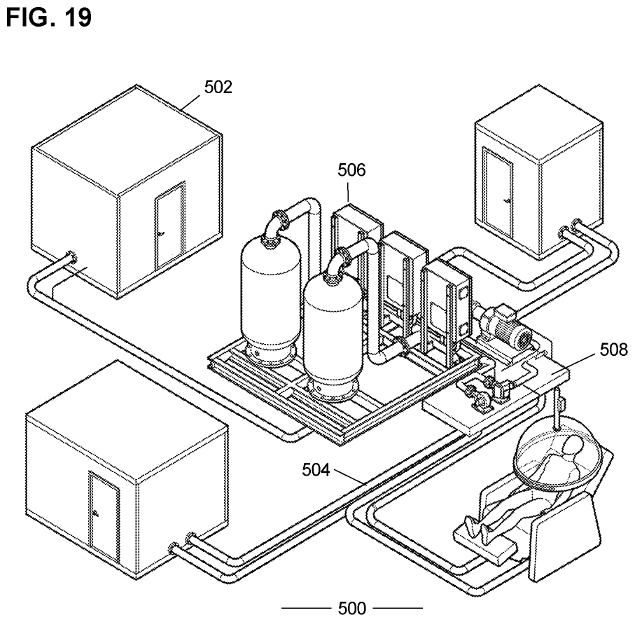

Referring now to FIG. 19, a representative site xenon utility system 500 includes a plurality of xenon-enabled spaces 502, a plurality of local capture paths, delayed local environmental recapture paths, facility capture paths, or combinations thereof 504 associated with the plurality of xenon-enabled spaces 502, a central or semi-central subsystem 506, and one or more source vessels, source manifolds, or exchange-vessel interfaces 508 configured to deliver xenon-bearing gas back to one or more of the xenon-enabled spaces 502. In certain embodiments, the central or semi-central subsystem 506 performs regeneration, concentration, purification, certification, blending, storage, redistribution, or combinations thereof.

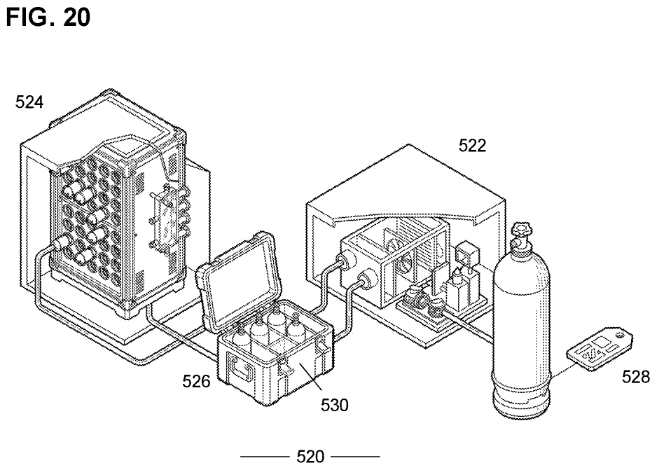

Referring now to FIG. 20, a representative distributed xenon recovery ecosystem 520 includes a first site 522 comprising one or more xenon recovery modules, a second site 524 comprising one or more regeneration or quality-assurance stations, a transport or handoff pathway 526, and a chain-of-custody subsystem 528 configured to manage one or more chain-of-custody states. In certain embodiments, one or more identity or state elements 530 are physically associated with one or more xenon recovery modules, transport housings, vessels, or combinations thereof.

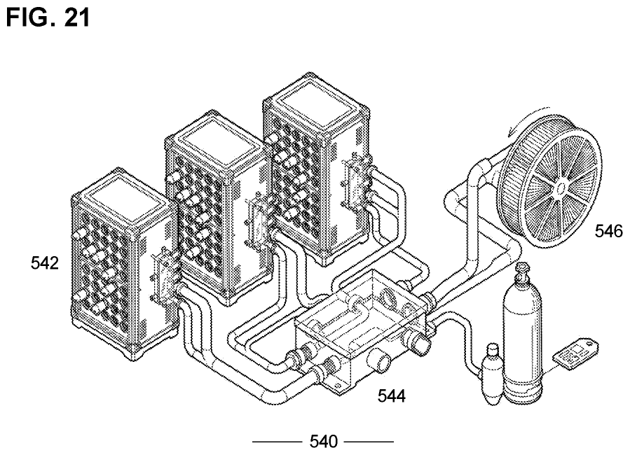

Referring now to FIG. 21, a representative continuous or quasi-continuous xenon recovery architecture 540 includes multiple xenon-selective modules 542 operated in staggered adsorption and regeneration states, a shared utility subsystem 544 configured to provide one or more of heat, vacuum, purge gas, cooling, or combinations thereof, and optionally a moving or rotating contactor 546. In certain embodiments, at least one module functions as a front-end collector and another functions as a secondary concentrator or microvolume collector.

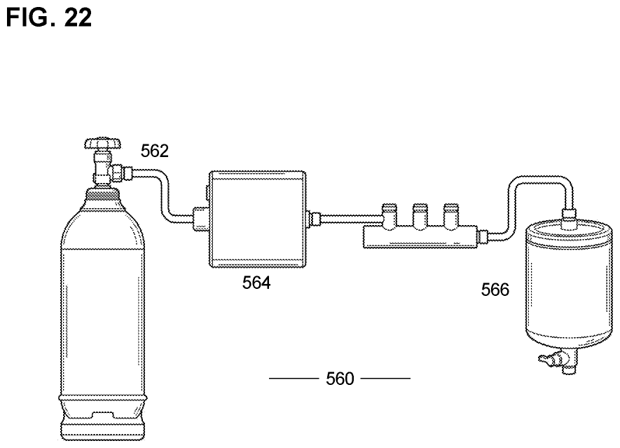

Referring now to FIG. 22, a representative local reuse workflow 560 includes a recovery path 562 configured to receive xenon-bearing gas from a local capture path, a delayed local environmental recapture path, a facility capture path, or combinations thereof, a quality-assurance or classification subsystem 564, and one or more physical destinations 566 associated with local reuse. In certain embodiments, recovered xenon-bearing gas is assigned to a local reuse class and is routed to a local reuse vessel, a local reuse manifold branch, a local reuse dispenser, a local reuse appliance inlet, or combinations thereof.

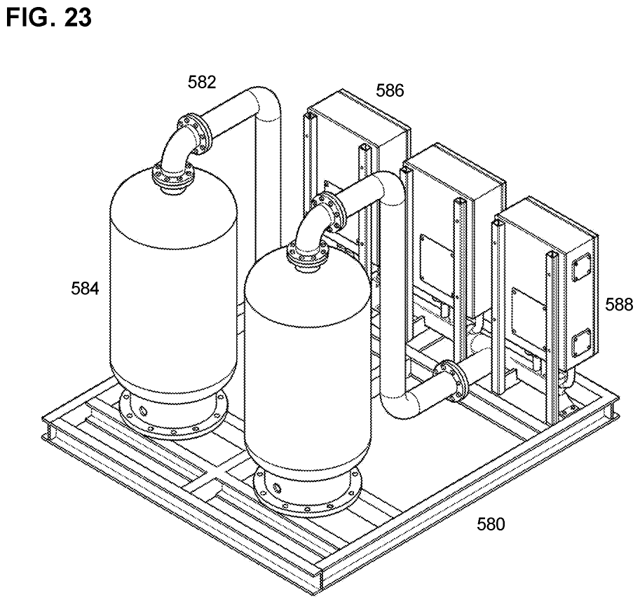

Referring now to FIG. 23, a representative centralized mechanical-room xenon recovery skid 580 includes one or more intake or return paths 582 coupled to room, duct, exhaust, or recirculation infrastructure, one or more xenon recovery modules or stages 584, one or more regeneration or xenon-release processing subsystems 586, and one or more downstream quality-assurance, storage, or blending subsystems 588. In certain embodiments, the skid 580 serves a plurality of treatment rooms, bays, operatories, or other xenon-enabled spaces. In certain embodiments, the regeneration subsystem 586, gas-moving subsystem, vacuum subsystem, compressor, or combinations thereof are positioned in a mechanical room, utility closet, basement, adjacent room, rooftop enclosure, or separate building that is physically separated from the administration environment, such that noise, vibration, heat rejection, or other operational disturbances generated by regeneration or gas-processing equipment are substantially isolated from the user, patient, or occupant during administration. In certain embodiments, gas piping, flexible conduits, penetration seals, vibration-isolating mounts, resilient hangers, acoustic enclosures, acoustic lagging, or combinations thereof are used to convey gas between the administration environment and the remotely located equipment while maintaining acceptable noise, vibration, and environmental conditions in the administration environment. In certain embodiments, the physical separation between the administration environment and the regeneration or gas-processing equipment is at least approximately 3 meters, or at least approximately 10 meters, or at least approximately 30 meters, or is separated by at least one wall, floor, ceiling, or building envelope, such that acoustic transmission and vibration transmission are attenuated to levels acceptable for clinical, wellness, or comfort-centered administration environments.

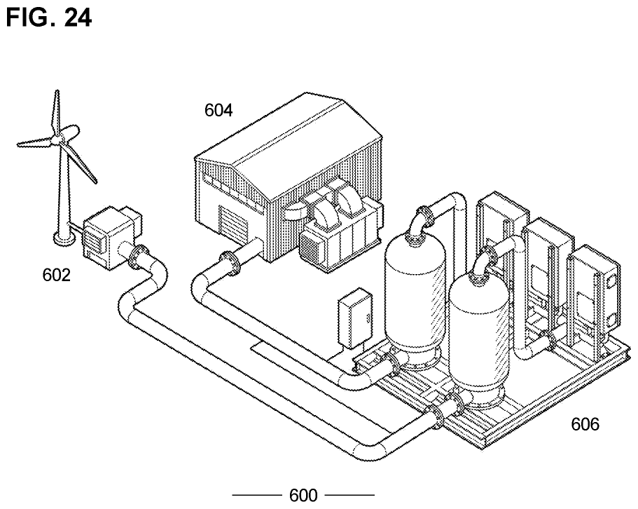

Referring now to FIG. 24, a representative hybrid deployment 600 includes at least one wind-powered capture node 602, at least one facility recapture node 604, and a shared downstream subsystem 606 configured for regeneration, concentration, certification, storage, blending, redistribution, or combinations thereof. In certain embodiments, xenon-bearing fractions from different feed regimes are aggregated, kept separate by source class, or selectively combined based on one or more quality attributes, compatibility classes, intended-use classes, or combinations thereof.

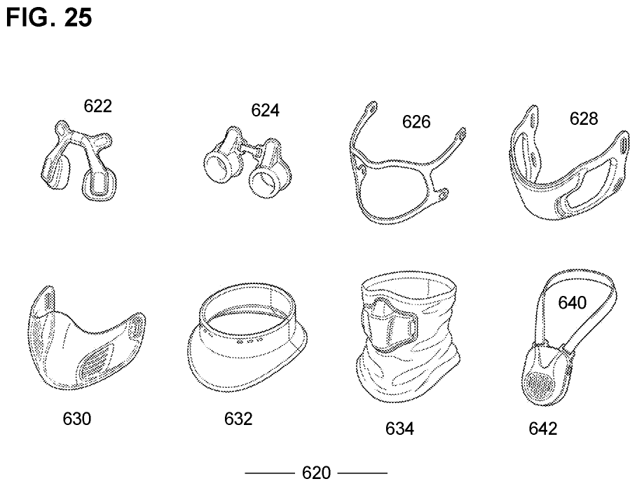

Referring now to FIG. 25, a representative personal interface family 620 includes one or more nasal interfaces 622, nostril interfaces 624, circumoral frames 626, partial face structures 628, transparent lower-face shells 630, collar-like boundaries 632, gaiter-integrated interfaces 634, and pendants 640 or chest pods 642, or combinations thereof. In certain embodiments, scarf-, wrap-, or textile-integrated variants are also contemplated and may be omitted from the figure for clarity. In certain embodiments, any member of the family is configured as a personal delivery interface, a local capture subsystem, an exhalation receiver, a sensing structure, or a hybrid thereof.

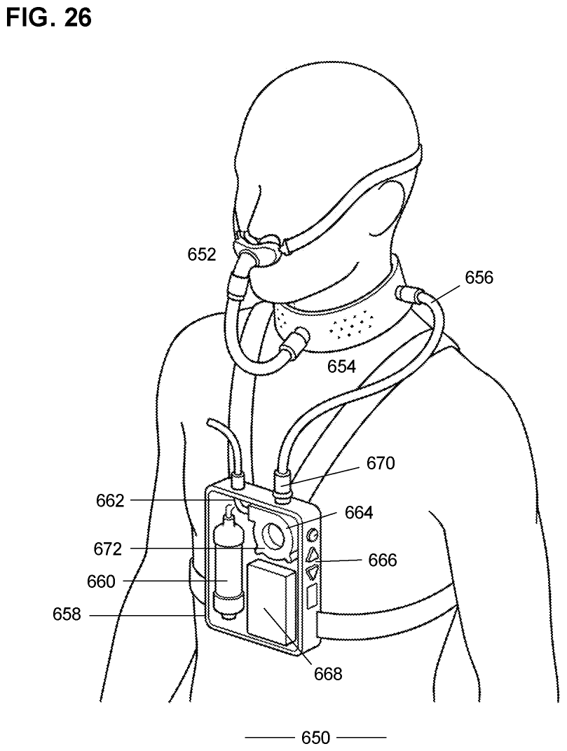

Referring now to FIG. 26, a representative distributed body-worn xenon system 650 includes a face-adjacent delivery interface 652, a local capture subsystem 654 positioned adjacent a neck, collar, or chest region, one or more flexible conduits 656, and a body-worn or user-carried subsystem 658 containing one or more of a source vessel 660, xenon recovery module 662, gas mover 664, controller 666, power subsystem 668, dehumidification subsystem 670, or regeneration subsystem 672. In certain embodiments, the system 650 establishes a personal breathing microenvironment adjacent a user's face while shifting heavier subsystems away from the face to improve comfort, thermal management, mobility, and wearability.

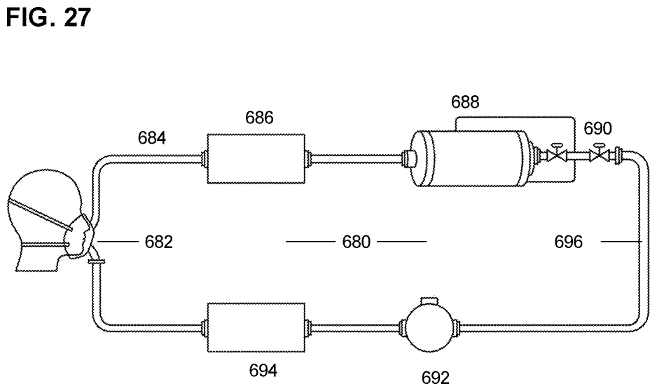

Referring now to FIG. 27, a representative personal xenon recovery loop 680 includes a personal breathing microenvironment 682, an interception or exhalation-receipt path 684, a dehumidification subsystem 686, a xenon-selective recovery subsystem 688, a portable or dockable regeneration subsystem 690, a reduced-volume collection region 692, an optional humidification subsystem 694 configured to return recovered moisture to delivered gas, and a gas mover 696 configured to circulate gas through the recovery loop. In certain embodiments, recovered xenon is routed to immediate reuse, blending, temporary storage, recirculation, or combinations thereof.

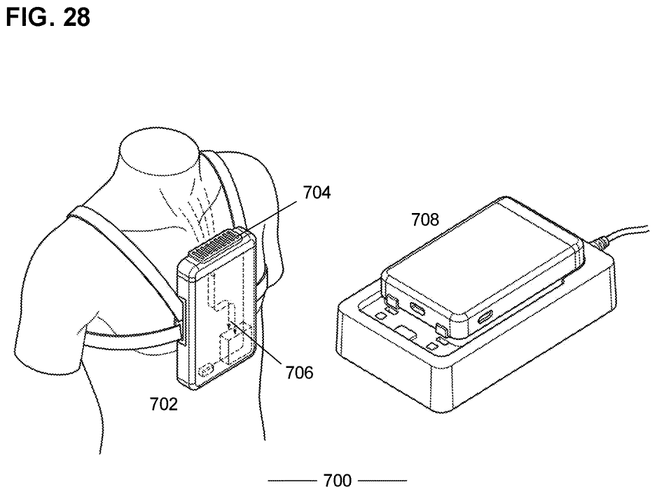

Referring now to FIG. 28, a representative personal exhalation-receiver embodiment 700 includes a chest pod, pendant, collar receiver, or similar local receiver 702 configured to receive passive or user-directed exhalation through one or more receiver inlets 704 and to route received gas through a conduit or internal flow path 706 toward xenon recovery or conditioning hardware. The receiver 702 may be docked with a cradle, station, or bedside unit 708 configured for charging, regeneration, refill, data exchange, synchronization, or combinations thereof. In certain embodiments, the local receiver 702 is removable from the dock 708 for ambulatory use and later reconnected for service or reuse.

### General Platform Overview

In certain embodiments, the invention comprises a xenon platform configured to obtain xenon from one or more xenon-containing gas mixtures and to route such xenon through one or more stages of capture, enrichment, regeneration, purification, storage, blending, delivery, recapture, or reuse. The platform is expressly multi-regime and is not limited to capture from true outdoor ambient air alone. Rather, the platform may receive, process, or otherwise interact with one or more of true ambient air, conditioned ambient air, recirculated air, return air, semi-enclosed or locally enriched atmospheres, scavenging gas, anesthesia waste gas, exhaled gas, purifier flow, ventilation return flow, exhaust flow, industrial off-gas, or other xenon-containing gas mixtures.

In certain embodiments, the platform is embodied as a standalone outdoor unit. In certain embodiments, it is embodied as a modular indoor or outdoor harvester. In certain embodiments, it is integrated with room recirculation, facility return air, purifier loops, or process waste streams. In certain embodiments, it is coupled to a regeneration station, facility recapture infrastructure, or a comfort-first administration environment. In certain embodiments, different feed regimes are processed by a common concentration core and common downstream quality-assurance, storage, or blending infrastructure, even though front-end capture geometry or pretreatment differs by regime.

### Controller Modes and Operational States

In certain embodiments, the controller is configured to operate the xenon capture platform in a plurality of operational modes, including without limitation a capture mode (also referred to as an adsorption mode or first mode) in which one or more xenon-selective stages are actively contacting a xenon-containing gas mixture, a regeneration mode (also referred to as a release mode or second mode) in which one or more xenon-selective stages are undergoing xenon-release processing, a cooling mode in which a recently regenerated stage is cooled toward adsorption temperature, a standby mode in which the system is in an idle or low-power state awaiting a trigger condition, a purge mode in which residual void gas is evacuated from one or more stages or manifold segments, a collection mode in which a xenon-bearing fraction is routed to a collection region or downstream concentrator, and a diagnostic mode in which one or more sensors, analyzers, or self-test routines evaluate system state. In certain embodiments, the controller transitions between modes based on one or more measured or inferred signals, including without limitation elapsed time, bed temperature, desorption temperature ramp, pressure, xenon concentration (measured or estimated), breakthrough detection, saturation estimation, demand signal, safety signal, or environmental condition. In certain embodiments, the controller implements a state machine, a rule-based scheduler, a model-predictive controller, or a hybrid thereof to manage mode transitions across two or more beds, modules, or stages. In certain embodiments, the transition between modes is actuated by one or more solenoid valves, pneumatic actuators, motor-driven valves, rotary valve assemblies, or electronically controlled flow diverters.

### Module Interface and Coupling Structures

In certain embodiments, a xenon recovery module, capture module, or other removable or serviceable element includes an interface configured to couple the module to a regeneration station, extraction station, service station, capture path, or other system element. In certain embodiments, the interface comprises one or more fluidic ports (such as threaded NPT fittings, quick-disconnect couplers, barbed fittings, O-ring sealed flanges, or compression fittings) configured to convey gas into or out of the module under capture, regeneration, or transfer conditions. In certain embodiments, the interface further comprises one or more mechanical engagement features such as keyed geometries, alignment pins, latch mechanisms, bayonet-style connectors, rail-and-slide assemblies, or magnetic alignment aids configured to ensure correct orientation, prevent misalignment, and facilitate repeatable docking. In certain embodiments, the interface further comprises one or more electrical, electronic, optical, or radio-frequency contacts configured to communicate identity, state, saturation history, regeneration count, certification status, or other data between the module and the station. In certain embodiments, the interface is compatibility-controlled such that a module of one class (for example, a medical-grade module) cannot be physically or logically coupled to a station or vessel of an incompatible class. In certain embodiments, the interface is designed for tool-free engagement and disengagement by a user, technician, or robotic handler, enabling rapid module swap without requiring specialized tooling or training.

### Feed Regimes

In many embodiments, the xenon capture platform operates across four principal feed regimes: true ambient air; conditioned ambient air; recirculated, semi-enclosed, or locally enriched atmospheres; and higher-concentration waste or process streams.

In true ambient air, xenon is present at atmospheric background concentration. In conditioned ambient air, the air remains atmosphere-derived but has been dried, filtered, cooled, compressed, pressure-conditioned, or otherwise modified before xenon-selective capture. In recirculated, semi-enclosed, or locally enriched atmospheres, xenon has already been intentionally introduced into a space or has been retained in a controlled environment such that xenon concentration exceeds ordinary atmospheric background. In higher-concentration waste or process streams, xenon is present at materially elevated concentration, for example in anesthesia waste gas, scavenging gas, exhaled-gas pathways, nonrecirculating exhaust, industrial off-gas, or other process flows. In certain embodiments, higher-concentration waste or process streams include krypton-xenon enriched streams from cryogenic or noncryogenic air-separation plants, noble-gas process tail gas, or other industrial gas streams.

The same physical and economic logic does not apply equally to all feed regimes. In true ambient air, the platform may be working-capacity-limited, humidity-limited, and dilution-limited. In locally enriched or waste-stream regimes, the same concentration core may become substantially more productive because xenon partial pressure is materially higher and the total air volume per unit xenon recovered is materially lower. The present disclosure therefore treats these regimes as members of one platform family rather than as separate inventions.

In true ambient-air embodiments, the amount of xenon recovered per cycle may be limited not only by the amount of xenon passing through the system, but also by the working capacity of the xenon-selective separation stage at the prevailing xenon partial pressure, by humidity and carbon-dioxide competition, by regeneration completeness, and by dilution in downstream void volumes. Accordingly, in many embodiments, a first atmospheric stage functions as a primary collector or primary enrichment stage that produces a loaded module or a xenon-enriched gas for later concentration, polishing, blending, certification, or reuse, rather than a final product in a single pass. This platform therefore expressly includes atmospheric embodiments in which throughput, pretreatment, stage count, or cycle design is selected to trade off yield, purity, energy use, and operational convenience without requiring that any one atmospheric stage alone provide a final product. In certain atmospheric embodiments, capture may be opportunistic, intermittent, campaign-based, or threshold-eligible and may aggregate xenon across multiple modules, multiple cycles, multiple days, or multiple nodes before later regeneration, concentration, polishing, certification, or reuse.

Any centrifugal, gravitational, swirl-induced, or stratification-based enrichment effect described herein may serve as an auxiliary enrichment, routing, or feed-conditioning mechanism and need not constitute the sole or primary xenon separation mechanism.

### Pretreatment and Feed Conditioning

In certain embodiments, the platform includes a pretreatment subsystem positioned upstream of, between, or downstream of one or more xenon-selective stages. The pretreatment subsystem may be configured to reduce or manage one or more of particulate matter, aerosols, droplets, biological contaminants, volatile organic compounds, reactive gases, sulfur compounds, ozone, nitrogen oxides, carbon monoxide, water, carbon dioxide, hydrocarbons, or combinations thereof. Pretreatment may include one or more filters, cyclones, inertial separators, electrostatic precipitators, coalescers, sorption beds, membrane dryers, refrigeration or condensation stages, desiccant wheels, regenerable desiccant cartridges, layered guard beds, reactive scrubbers, catalytic converters, or combinations thereof.

In many embodiments, moisture management is treated as enabling architecture rather than as an optional refinement. This is particularly true for true ambient-air capture and other dilute-feed embodiments, where water can compete strongly with xenon for active sites, alter breakthrough behavior, reduce working capacity, increase regeneration burden, and increase impurity load in the desorbed fraction. Accordingly, in certain embodiments the pretreatment subsystem is configured to produce a partially dried, substantially dried, or deeply dried feed prior to contact with a xenon-selective stage. In certain embodiments, a water-removal stage is paired with a carbon-dioxide guard stage, either sequentially or in an integrated layered module.

The sensitivity of many xenon-selective sorbents to water is quantitatively significant. Published data on silver-exchanged zeolites indicate that water co-adsorption at relative humidities of approximately 40 to 60 percent can reduce xenon working capacity by a factor of approximately 10 to 40 or more compared to dry conditions, depending on the zeolite framework, silver loading, and exposure history. In some materials, water loading can cause previously adsorbed xenon to be displaced in a phenomenon sometimes termed roll-up, where a transient increase in xenon effluent concentration is observed as advancing water displaces xenon from active sites. This behavior underscores the importance of upstream moisture management in embodiments targeting high xenon recovery from humid feeds.

In certain embodiments, the pretreatment subsystem is configured to reduce the water content of the feed gas to below approximately 100 parts per million by volume, or below approximately 10 parts per million, or below approximately 1 part per million, prior to contact with a xenon-selective stage, with the target moisture level selected according to the sensitivity of the chosen xenon-selective medium and the acceptable impact on xenon working capacity. In certain embodiments, the pretreatment subsystem includes one or more of a condensation stage, a desiccant wheel, a regenerable desiccant bed, a membrane dryer, a refrigeration dryer, or combinations thereof, and may employ waste heat, solar thermal energy, or recovered regeneration heat to manage the energy cost of drying. In certain embodiments involving enriched-feed or recirculated-atmosphere capture, the moisture burden may be further managed by a carbon dioxide scrubber that also removes water, or by a combined CO2-and-moisture management module positioned upstream of the xenon-selective stage.

In some embodiments, pretreatment is itself staged and regenerable. For example, a first layer may reduce particulate loading, a second layer may remove or reduce water, a third layer may remove or reduce carbon dioxide or other competing species, and a fourth layer may act as a sacrificial or protective zone to preserve the performance of a xenon-selective stage. The invention expressly contemplates pretreatment cartridges or beds that are removable, reloadable, serviceable, permanently installed, field-regenerated, off-site regenerated, or regenerated automatically in situ.

The thermodynamic significance of dehydration in dilute-feed xenon capture merits particular emphasis. At standard conditions of approximately 25 degrees Celsius and approximately 50 percent relative humidity, the water content of atmospheric air is approximately 12 to 16 grams per cubic meter, compared to a xenon content of approximately 0.5 milligrams per cubic meter. The mass ratio of water to xenon in ambient air is therefore approximately 25,000 to 30,000 to one. At a processing rate of approximately 1,000 cubic feet per minute, the water removal rate required to produce deeply dried feed gas may be approximately 15 to 25 kilograms per hour, representing a thermal load of approximately 10 to 20 kilowatts depending on the dehydration depth and staging arrangement. This dehydration energy load may exceed the energy required for xenon-selective adsorption and regeneration by a factor of approximately 30 to 100 or more, depending on the feed humidity, target dew point, and regeneration strategy. Dehydration is therefore not merely a pretreatment convenience but rather the thermodynamically dominant subsystem in dilute-feed xenon capture, and the energy management of dehydration is a primary determinant of overall system economics.

In certain embodiments, the dehydration subsystem employs a cascaded multi-stage architecture in which a first refrigeration or condensation stage removes the bulk of the water load to a dew point of approximately 2 to 10 degrees Celsius, a second desiccant or desiccant-wheel stage further reduces moisture to a dew point of approximately minus 20 to minus 40 degrees Celsius, and an optional third molecular-sieve stage achieves deep drying to a dew point of approximately minus 60 to minus 80 degrees Celsius, as required by the sensitivity of the selected xenon-selective medium. This cascaded approach is thermodynamically more efficient than single-stage deep drying because each stage operates across a smaller humidity differential and can be optimized independently for energy input, regeneration temperature, and duty cycle.

In certain embodiments, the dehydration subsystem is thermally integrated with the xenon regeneration subsystem such that waste heat generated during xenon sorbent regeneration—typically at temperatures of approximately 150 to 250 degrees Celsius—is cascaded to supply thermal energy for desiccant regeneration, which typically requires lower temperatures of approximately 80 to 150 degrees Celsius. In certain embodiments, this thermal cascade reduces the total external energy input for dehydration by approximately 20 to 60 percent compared to a system in which the dehydration subsystem is powered entirely by external energy. In certain embodiments, the scheduling of xenon-selective regeneration cycles and desiccant regeneration cycles is coordinated by a controller to maximize waste-heat utilization, for example by performing desiccant regeneration during or immediately after xenon sorbent regeneration so that high-grade waste heat from the xenon sorbent is available to the desiccant stage. In certain embodiments, rich-loop recirculation sessions produce concentrated waste heat that may be time-shifted to support ambient-harvesting dehydration during or after administration sessions, thereby coupling the two operating modes in a thermodynamically synergistic schedule.

The dehydration subsystem disclosed herein expressly contemplates a broad genus of moisture-removal technologies, either individually or in any staged combination, including without limitation: refrigerative condensation using vapor-compression or absorption-cycle heat pumps operating at coefficients of performance greater than approximately 2.5; solid desiccant adsorption using silica gel, activated alumina, molecular sieves (including 3A, 4A, and 13X zeolites), metal-organic frameworks (including MOF-303, MOF-801, and related water-harvesting frameworks), or other regenerable solid desiccants in fixed beds, rotating wheels, or desiccant-coated heat exchangers; liquid desiccant absorption using hygroscopic solutions such as lithium chloride, calcium chloride, triethylene glycol, or other liquid desiccants with regeneration at temperatures of approximately 45 to 70 degrees Celsius; membrane drying using water-selective polymeric membranes such as Nafion, sulfonated polyethersulfone, hollow-fiber membrane modules, flat-sheet membrane modules, or isothermal membrane-based air dehumidification (IMAD) configurations capable of achieving dew points of approximately minus 40 to minus 60 degrees Celsius; electrochemical or electrolytic dehumidification using proton-exchange-membrane (PEM) cells or electrohydrodynamic (EHD) enhanced condensation; thermoelectric or Peltier-cooled condensation stages; acoustic or ultrasonic droplet coalescence operating at frequencies of approximately 300 hertz to ultrasonic ranges to enhance droplet capture ahead of inertial separators; inertial separation including cyclonic, vortex, chevron-baffle, or wave-plate mist eliminators; supersonic or expansion-based dehumidification using Laval nozzle or Joule-Thomson expansion to achieve rapid cooling and condensation; vortex tube (Ranque-Hilsch) dehydration; passive or radiative cooling condensation; deliquescent salt beds for zero-energy passive bulk moisture reduction; heat-pump-assisted liquid desiccant (HP-LDD) systems; active membrane energy exchangers (AMX) with cross-flow cooling; pressure-swing or vacuum-swing drying cycles; and any hybrid or cascaded combination of the foregoing.

In certain embodiments, the dehydration subsystem is selected or adapted according to the prevailing environmental conditions, feed regime, available energy source, and target dew point. In arid or cold environments, one or more upstream dehydration stages may be omitted or reduced in capacity. In tropical or humid environments, additional staging depth or higher-capacity dehydration hardware may be employed. In certain embodiments, a controller selects among available dehydration modes or adjusts the operating setpoints of one or more dehydration stages based on measured or predicted inlet humidity, ambient temperature, energy availability, or xenon demand. In certain embodiments, the dehydration subsystem is modular such that individual stages may be added, removed, bypassed, or replaced according to site conditions, seasonal variation, or technology upgrade without modifying the downstream xenon-selective capture, regeneration, or administration subsystems.

Because dehydration is the dominant energy load, advances in dehydration technology—including but not limited to improved desiccant materials, more efficient membrane dryers, lower-energy refrigeration cycles, electrochemical dehumidification, heat-pump-assisted drying, or novel moisture-selective media—are expected to directly and proportionally improve the overall energy efficiency and economic viability of xenon capture from humid ambient air. The dehydration architecture disclosed herein is designed to accommodate such future improvements without requiring changes to the downstream xenon-selective stages, regeneration workflows, recapture strategies, or administration environments.

### Carbon Dioxide Management

In certain embodiments, carbon dioxide management is treated as a second enabling pretreatment architecture alongside dehydration. Atmospheric air contains carbon dioxide at approximately 420 parts per million by volume, and many xenon-selective adsorbent materials also exhibit appreciable CO₂ adsorption capacity, such that carbon dioxide can compete with xenon for active sites, reduce xenon working capacity, and appear as an impurity in the desorbed xenon-bearing fraction. In enriched-feed or recirculated-atmosphere regimes, metabolic CO₂ from human respiration may be present at concentrations of approximately 2 to 5 percent by volume, representing a substantially greater competitive burden than atmospheric levels. In certain embodiments, the platform therefore includes one or more carbon dioxide management stages, which may be positioned upstream of the xenon-selective stage, between sequential xenon-selective stages, within a recirculation loop, or as a downstream polishing step.

Carbon dioxide management stages may include without limitation: soda lime or other hydroxide-based chemical scrubbers; regenerable amine scrubbers; solid CO₂ adsorbents such as zeolite 13X, activated alumina, hydrotalcites, amine-functionalized silica, or amine-functionalized metal-organic frameworks; CO₂-selective membranes including polymeric, facilitated-transport, or mixed-matrix membranes; CO₂-selective porous materials such as CUPMOF-5, DD3R, or related frameworks that preferentially retain or transmit CO₂ relative to xenon; pressure-swing or vacuum-swing CO₂ removal; cryogenic CO₂ condensation or anti-sublimation; electrochemical CO₂ separation; or combinations thereof.

In certain embodiments, CO₂ management is integrated with dehydration in a layered guard-bed arrangement such that a first region removes water and a second region removes carbon dioxide, or a combined sorbent simultaneously manages both species. In certain embodiments involving recirculated or enriched atmospheres, a CO₂-selective material that preferentially retains CO₂ while allowing xenon to pass through—such as certain narrow-pore zeolites, DDR-type membranes, or CO₂-philic MOFs—may be employed as an alternative or complement to xenon-selective capture, effectively purifying xenon by removing the contaminant rather than by capturing the product. This “keep xenon, remove CO₂” approach is an alternative architectural strategy that may be advantageous in enriched-feed regimes where xenon constitutes a substantial fraction of the gas stream.

### Xenon-Selective Separation Stages and Structured Contactors

In certain embodiments, xenon is captured, enriched, slowed, partitioned, retained, or otherwise selectively handled in one or more xenon-selective separation stages. Such stages may be adsorption stages, absorption stages, membrane stages, condensation stages, freezing stages, chromatography stages, diffusion-based stages, field-assisted stages, electrothermal release stages, or combinations thereof. In preferred embodiments, one or more adsorption stages are used because of their modularity and suitability for cyclical capture and regeneration; however, the invention is not limited to adsorption-first embodiments and expressly includes membrane-first, hybrid adsorption-membrane, adsorption-cryogenic, adsorption-chromatographic, and other multi-mode combinations.

In some embodiments, one or more xenon-selective separation stages may be membrane-based, cryogenic, condensation-based, freezing-based, diffusion-based, chromatographic, or hybrid, either as a primary stage, a secondary concentrator, or a polishing stage. In certain embodiments, an adsorption stage performs a first enrichment and a membrane or cryogenic stage performs a later polishing or class-specific product preparation step. In certain embodiments, a membrane, cryogenic, or other non-adsorptive stage may instead precede an adsorption stage or operate in parallel with an adsorption stage. The invention is therefore not limited to adsorption-first workflows and expressly includes staged combinations of adsorptive and non-adsorptive xenon-selective separation stages.

The xenon-selective medium may comprise any suitable xenon-selective material or later-developed material class, including without limitation zeolites, silver-exchanged zeolites, non-silver zeolites, cation-exchanged zeolites, chabazites, ETS-family materials, silica-aluminophosphates, metal-organic frameworks, hydrogen-bonded frameworks, porous organic cages, covalent organic frameworks, porous carbons, activated carbon, carbon molecular sieves, mixed-matrix materials, supported nanoparticles, structured composite media, polymeric membrane materials, microporous crystalline membranes, or combinations thereof. Non-limiting preferred examples for disclosure purposes include Ag-SSZ-13, Ag-ZSM-5, Ag-ETS-10, natural or synthetic chabazite variants, SBMOF-1 and structured SBMOF-1 embodiments, transition-metal-free zeolite variants, and SAPO-34 or related membrane stages. No claim to the broader system is intended to require any one named material unless explicitly recited. Because the system architecture, process workflows, staged concentration cascades, fraction-cut collection methods, regeneration protocols, recapture strategies, and administration environments disclosed herein are defined primarily by their functional and thermodynamic relationships rather than by the identity of any particular sorbent, membrane, or separation medium, the disclosed systems and methods are expressly intended to encompass xenon-selective materials that are subsequently discovered, developed, computationally designed, or otherwise identified after the filing date of this application, including without limitation materials identified through machine-learning-guided or artificial-intelligence-guided materials discovery, high-throughput computational screening, combinatorial synthesis, post-synthetic modification of known frameworks, or other future materials-development methodologies. As the performance of xenon-selective materials improves—for example, through higher working capacities, faster kinetics, lower regeneration temperatures, improved moisture tolerance, higher selectivity, or longer service life—the systems and methods disclosed herein are expected to become correspondingly more productive, more energy-efficient, more compact, and more economically favorable, because the architecture imposes no upper limit on the performance of the xenon-selective medium and is designed to benefit from any such improvement without requiring structural modification of the system, process, or workflow architecture.

The xenon working capacity of an adsorbent material—defined as the usable difference in xenon loading between adsorption and regeneration conditions—depends strongly on xenon partial pressure, temperature, humidity, co-adsorption of other species, and regeneration completeness. For silver-exchanged zeolites such as Ag-SSZ-13, Ag-ZSM-5, and Ag-ETS-10, published adsorption data indicate equilibrium xenon capacities in the range of approximately 0.1 to 2.5 millimoles per gram under dry conditions at room temperature, with the actual value depending on xenon partial pressure, silver loading, zeolite framework topology, and activation conditions. At the very low xenon partial pressures characteristic of true ambient air at approximately 87 parts per billion, working capacity may be in the Henry's law regime, where loading scales approximately linearly with partial pressure, and practical working capacities on the order of approximately 0.01 to 0.3 millimoles per kilogram of sorbent may be expected for well-prepared silver-exchanged zeolites under dry conditions, with the wide range reflecting differences in Henry's law constants among candidate materials, silver loading, framework topology, and activation history. This extremely low per-cycle loading is a direct consequence of the ultra-dilute feed and is the fundamental driver of the high air-throughput requirement. At the intermediate xenon partial pressures characteristic of partially enriched streams, such as approximately 1 to 100 parts per million, working capacity increases approximately in proportion to the partial pressure increase, reaching on the order of 1 to 30 millimoles per kilogram under dry conditions. At the higher xenon partial pressures characteristic of recirculated or enriched atmospheres, such as approximately 0.1 to 50 percent by volume, working capacities may approach or reach several millimoles per gram, depending on material choice and operating conditions, as the isotherm transitions from the Henry's law regime toward a Langmuir plateau.

In certain embodiments, a xenon-selective bed comprising approximately 5 to 20 kilograms of active sorbent material in a structured contactor geometry may yield approximately 10 to 50 milliliters of xenon at standard temperature and pressure per adsorption-regeneration cycle when operating on true ambient air, and substantially more when operating on enriched-feed streams. In certain embodiments, the system is configured to perform approximately 2 to 12 adsorption-regeneration cycles per hour, with cycle duration adapted to feed concentration, bed size, and acceptable breakthrough fraction. Selectivities of xenon over nitrogen, oxygen, argon, and other major air components may range from approximately 100 to over 4000, depending on material class and conditions, with silver-exchanged zeolites generally exhibiting higher selectivities than non-silver alternatives at low xenon partial pressures.

The isosteric heat of xenon adsorption on silver-exchanged zeolites is typically in the range of approximately 35 to 50 kJ per mole, which determines the thermal energy input required for regeneration and influences the temperature differential needed for effective thermal swing operation. In certain embodiments, the regeneration temperature for zeolite-based media is selected in the range of approximately 150 to 250 degrees Celsius, with deeper regeneration at higher temperatures yielding more complete xenon release but requiring more energy input per cycle.

In certain embodiments, the xenon-selective medium is embedded in or supported by a structured contactor rather than a simple bulk powder bed. Non-limiting examples include trays, meshes, woven screens, perforated sheets, foils, laminates, heat-exchanger plates, pleated sheets, monoliths, honeycombs, radial-flow cartridges, axial-flow cartridges, rotating wheels, drums, moving beds, beads, pellets, fibers, felts, cloths, foams, washcoated substrates, coated metal supports, coated ceramic supports, coated carbon supports, or combinations thereof. In certain embodiments, the support is substantially thermally conductive to improve heating and cooling during swing operation. In certain embodiments, the support is substantially electrically conductive such that it may function as, or in conjunction with, a resistive heating element. In certain embodiments, multiple contactor geometries are combined in series, parallel, nested, or layered arrangements.

The selection of structured contactor geometry involves a trade-off between pressure drop, mass-transfer rate, adsorbent density, thermal management, and manufacturability. In high-throughput embodiments processing hundreds or thousands of cubic feet per minute, pressure drop across the xenon-selective stage may be a dominant contributor to overall energy consumption. For a packed bed of granular media, pressure drop per unit length scales approximately inversely with the square of the particle diameter in the viscous-flow regime, as described by established correlations such as the Ergun equation. This scaling means that fine granular media offering high mass-transfer rates also impose high pressure drops, which can dominate fan energy consumption at elevated flow rates.

Structured contactors such as monoliths, laminates, and parallel-channel geometries offer substantially lower pressure drop per unit of adsorbent mass compared to packed granular beds, because gas flow is directed through open channels rather than through a tortuous interstitial path. In certain embodiments, a structured contactor achieves a pressure drop of less than approximately 500 pascals, or less than approximately 200 pascals, or less than approximately 50 pascals, at the design flow rate, while maintaining sufficient xenon-selective surface area and mass-transfer rate for the intended cycle time. In certain embodiments, the structured contactor support is thermally conductive, enabling rapid heating during regeneration and rapid cooling during the return to adsorption mode, which may reduce cycle dead time and improve average productivity.

While the preferred embodiments utilize selective adsorption on microporous sorbents (e.g., silver-exchanged zeolites), the xenon recovery system is not limited to adsorptive separation. In a membrane-based embodiment, the separation module comprises a polymeric or mixed-matrix membrane exhibiting a xenon-to-oxygen selectivity of at least 5 and a xenon permeance of at least 50 GPU (gas permeation units), operated at a transmembrane pressure ratio between 2:1 and 6:1. A sweep gas on the permeate side carries the xenon-enriched fraction to the collection vessel. In a cryogenic micro-trap embodiment, the separation module comprises a compact cold surface maintained at 120 to 150 K with an internal volume of less than 100 mL, onto which xenon preferentially condenses from a pre-dried feed stream while lighter gases (N₂, O₂, Ar) pass through. These non-adsorptive embodiments may be used independently or in combination with adsorptive stages in a hybrid separation architecture, broadening the genus of separation mechanisms claimed herein.

### Adsorption-Mode Operation and Feed-Regime Adaptation

During adsorption mode, a xenon-containing gas mixture is brought into contact with one or more xenon-selective stages under conditions selected to promote selective xenon retention, enrichment, or other desired separation behavior. Gas motion may be provided by one or more fans, blowers, compressors, ejectors, pumps, turbines, facility returns, negative-pressure systems, positive-pressure systems, natural draft, or combinations thereof. The system may operate at ambient pressure, subambient pressure, modestly elevated pressure, cyclic pressure, or other selected pressure regimes. Temperature during adsorption may be ambient, subambient, actively conditioned, or derived from another system process such as cooling or heat exchange.

In true ambient-air embodiments, the system may be operated with shortened adsorption intervals, multiple parallel beds, or rapid swing sequences because such feeds may be working-capacity-limited rather than feed-limited. In such embodiments, the concentration core may be configured to switch a bed or module before substantial xenon breakthrough, thereby increasing average recovery over time. In recirculated-room, booth, hood, cabin, purifier-loop, HVAC-return, or waste-stream embodiments, the same concentration core may be operated at lower volumetric throughput and/or higher effective xenon partial pressure, enabling longer adsorption intervals, different regeneration timing, different purity targets, or different downstream handling strategies.

In certain embodiments, the system is eligibility-gated, meaning one or more actions are initiated, delayed, intensified, or inhibited based on measured or inferred system state. By way of example, adsorption may begin or continue only when at least one of xenon concentration, humidity, temperature, power availability, return-on-regeneration expectation, saturation state, or user demand meets a selected criterion.

### Thermodynamic Considerations and Feed-Regime Scaling

The thermodynamic constraints on xenon capture from gas mixtures are governed in part by the minimum reversible work of separation and the volumetric throughput required to contact sufficient gas. For an ideal gas mixture at environmental temperature and pressure, the minimum reversible work to separate a dilute component scales approximately as:

w_min≈RT₀ln(1/y)

where R is the ideal gas constant, T₀ is the environmental temperature, and y is the mole fraction of the target component. For xenon in true ambient air at approximately 87 parts per billion by volume, the minimum reversible work at approximately 298 K is approximately 40 kJ per mole of xenon recovered, corresponding to approximately 1.6 to 1.7 kJ per liter of xenon at standard temperature and pressure. This minimum decreases logarithmically as feed xenon concentration increases; for example, at approximately 1 part per million the minimum is approximately 34 kJ per mole, at approximately 100 parts per million the minimum is approximately 23 kJ per mole, and at approximately 1 percent the minimum is approximately 11 kJ per mole. In practice, the dominant energy expenditures in dilute-feed xenon capture are associated with moving and conditioning large volumes of air rather than with the reversible work of separation itself.

The volumetric throughput scaling is more constraining than the energy scaling for dilute-feed embodiments. At atmospheric xenon concentration of approximately 87 parts per billion, the volume of air containing one liter of xenon at standard conditions is approximately 11,500 cubic meters. This throughput requirement decreases linearly with increasing xenon concentration, such that localized environments with elevated xenon concentration—including recirculated atmospheres, exhaled-gas streams, scavenging streams, and enriched breathing zones—require orders of magnitude less air handling per unit of xenon recovered. This physical reality motivates the multi-regime architecture disclosed herein, in which the system may operate across a range of feed concentrations from true ambient through enriched or recirculated atmospheres, with appropriately adapted cycle parameters, bed sizing, regeneration timing, and downstream handling.

In certain embodiments involving true ambient-air capture at a blower capacity of approximately 500 to 1500 cubic feet per minute, the feed-limited maximum xenon production rate is approximately 1.5 to 5 liters per day at standard temperature and pressure, depending on blower capacity and capture efficiency. This production rate may be suitable for supplemental xenon inventory replenishment, idle-mode ambient harvesting, or low-demand applications. In certain embodiments involving enriched-feed or recirculated-atmosphere capture at xenon concentrations of approximately 0.1 to 50 percent by volume, the feed-limited production rate is orders of magnitude higher per unit of air handled, enabling economically favorable real-time recapture during administration sessions.

### Scalable and Geographically Optimized Ambient Harvesting

The thermodynamic and volumetric scaling relationships described above are not limited to room-scale or facility-scale embodiments. In certain embodiments, the xenon capture platform is scaled to process air at rates of approximately 5,000 to 10,000 cubic feet per minute, or approximately 10,000 to 100,000 cubic feet per minute, or greater than approximately 100,000 cubic feet per minute, using correspondingly larger or more numerous xenon-selective stages, pretreatment subsystems, regeneration subsystems, and downstream handling capacity. The minimum reversible work of separation scales logarithmically with feed dilution and is independent of system throughput, while the volumetric throughput requirement scales linearly with throughput, such that the fundamental thermodynamic architecture disclosed herein is valid at any practical scale. In large-scale ambient-harvesting embodiments, multiple parallel structured contactors, rotating-wheel contactors, moving-bed contactors, or banks of modular harvesters may be employed, with shared pretreatment, shared regeneration, shared thermal management, and shared downstream quality assurance.

In certain embodiments, the geographic location of an ambient-air xenon capture installation is selected to exploit favorable environmental conditions that reduce pretreatment burden, increase effective throughput, or improve energy economics. Environmental variables relevant to site selection include ambient humidity, ambient temperature, prevailing wind speed, wind consistency, atmospheric particulate loading, elevation, barometric pressure, solar irradiance, and availability of waste heat or renewable energy. In certain embodiments, arid or semi-arid environments—including desert regions, high-altitude plateaus, polar or subpolar zones, or other naturally dry climates—are preferred for ambient-air xenon capture because the reduced moisture burden substantially lowers dehydration energy, reduces or eliminates one or more pretreatment stages, and allows more of the available energy budget to be directed toward xenon-selective capture and regeneration rather than water removal. As disclosed herein, water co-adsorption can reduce xenon working capacity by a factor of approximately 10 to 40 or more; accordingly, locating an ambient-harvesting installation in a region where ambient relative humidity is below approximately 20 percent or where ambient dew point is below approximately minus 10 degrees Celsius may yield a proportional improvement in xenon production rate per unit of sorbent mass.

In certain embodiments, high-wind environments are preferred because sustained natural wind reduces or eliminates the energy required for active gas movement. In such embodiments, wind-driven airflow is directed through one or more xenon-selective stages using wind-catcher structures, wind towers, funnel inlets, converging duct geometries, venturi-assisted flow paths, or wind-turbine-integrated airflow paths as described elsewhere herein. In certain embodiments, a wind-driven ambient harvester operates with no active fan or blower power, deriving all gas-movement energy from prevailing wind. In certain embodiments, a wind-driven system supplements fan-driven flow during periods of sufficient wind velocity and reverts to powered operation during calm periods.

In certain embodiments, the combination of low humidity and high wind velocity creates a synergistic advantage for ambient-air xenon capture, because the low humidity reduces the dominant energy load (dehydration) while the high wind velocity reduces or eliminates the secondary energy load (gas movement). Regions exhibiting both characteristics—such as arid trade-wind zones, high-altitude desert plateaus, coastal desert corridors, and continental interior steppe environments—may therefore represent particularly favorable geographic positions for large-scale ambient xenon harvesting installations. In certain embodiments, site-selection criteria further include proximity to renewable energy resources (solar, wind, geothermal), proximity to xenon demand centers, transportation infrastructure, regulatory environment, and land cost.

In certain embodiments, the scalable ambient-harvesting architecture disclosed herein is expected to become increasingly economically favorable as xenon-selective adsorbent materials improve over time, because the energy and throughput scaling laws described herein amplify the benefit of higher working capacities and faster kinetics at larger scales. An adsorbent material with twice the working capacity of current materials would approximately halve the bed mass required at any given throughput, and the resulting cost and energy savings compound with scale.

### Comparative Energy Economics

The energy efficiency of the ambient xenon capture architecture disclosed herein may be compared against conventional cryogenic production methods to illustrate the magnitude of the thermodynamic advantage achievable through geographic optimization and low-pressure-drop structured contactor design.

In conventional industrial practice, xenon is produced exclusively as a minor co-product of large-scale cryogenic air separation units (ASUs) that liquefy air at temperatures of approximately 77 to 90 Kelvin and pressures of approximately 5 to 6 bar, primarily to produce oxygen and nitrogen. The energy required for xenon recovery from such a process depends on the allocation methodology. The marginal incremental energy consumed by the krypton-xenon recovery subsystem —that is, the additional energy beyond what the ASU would consume if it simply vented these trace gases—is estimated at approximately 2 to 27 kilowatt-hours per liter of xenon at standard temperature and pressure, based on vendor data for industrial krypton-xenon extraction modules and engineering estimates of the recovery column energy as approximately 0.1 to 1 percent of total ASU consumption. The full-allocation energy—the total ASU energy divided by xenon output, appropriate for comparison against a hypothetical dedicated xenon-from-air process—is estimated at approximately 500 to 2,500 kilowatt-hours per liter, reflecting the enormous energy required to cryogenically process the approximately 11.5 million liters of air that contain one liter of xenon at 87 parts per billion.

The minimum thermodynamic work of separation for xenon from air is governed by the expression W_min=RT ln(1/y_Xe), yielding approximately 40.3 kilojoules per mole or approximately 0.0005 kilowatt-hours per liter of xenon at standard temperature and pressure. Conventional cryogenic plants operate at approximately 4,000 to 40,000 times this thermodynamic minimum for xenon specifically, owing to the enormous irreversibility of cooling and liquefying the entire air stream to access a trace component.

In the ambient capture architecture disclosed herein, the total energy per liter of xenon at standard temperature and pressure is dominated by three components: (i) dehydration energy to achieve the moisture levels required for effective xenon-selective adsorption (dew point of approximately minus 73 degrees Celsius or below), (ii) mechanical energy to move the requisite air volume through the structured contactor, and (iii) thermal energy for sorbent regeneration. In temperate conditions (approximately 25 degrees Celsius, approximately 50 percent relative humidity), the reversible minimum dehydration energy alone is approximately 9.4 kilowatt-hours per liter of xenon at standard temperature and pressure, because the water-to-xenon molar ratio in humid air exceeds approximately 25,000 to 1, and this dehydration energy exceeds the reversible minimum for airflow work by a factor of approximately 8 to 10. In these temperate conditions, the total energy for ambient capture exceeds approximately 10 kilowatt-hours per liter, which is not competitive with marginal cryogenic recovery from an existing ASU.

However, in geographically optimized conditions—specifically at sites where the ambient temperature is below approximately minus 40 degrees Celsius such that the ambient dew point already approaches or meets the sorbent moisture protection threshold—the dehydration energy component is reduced by approximately 100 to 300 times or eliminated entirely. At locations where ambient ice-saturation vapor pressure is below approximately 0.16 Pascals (corresponding to ambient temperatures below approximately minus 75 degrees Celsius), the environment has effectively performed the deep dehydration, and the dominant remaining energy term is the mechanical work to move air through the structured contactor, which scales linearly with pressure drop and air volume. For structured monolith or laminate contactors with pressure drops of approximately 10 to 200 Pascals, the mechanical energy component at sub-ambient temperatures is approximately 0.04 to 1.2 kilowatt-hours per liter of xenon at standard temperature and pressure. The thermal regeneration energy for xenon desorption (using an isosteric heat of adsorption of approximately 40 kilojoules per mole) contributes approximately 0.0005 to 0.001 kilowatt-hours per liter of xenon at standard temperature and pressure, which is negligible relative to the mechanical work at ambient-capture scale.

At wind-driven sites where prevailing wind velocities of approximately 15 to 22 meters per second generate dynamic pressures of approximately 150 to 340 Pascals—exceeding the pressure drop of the structured contactor—the mechanical airflow energy is derived entirely from wind, reducing the purchased energy to only the thermal regeneration component and parasitic control loads. Under these conditions, the total purchased energy for ambient xenon capture is predicted to be in the range of approximately 0.1 to 0.6 kilowatt-hours per liter of xenon at standard temperature and pressure, representing an improvement of approximately 5 to 500 times relative to marginal cryogenic recovery from an existing ASU, and approximately 1,000 to 7,000 times relative to dedicated cryogenic production from air. The energy per liter achieved at geographically optimized sites is comparable to that reported for vacuum-pressure-swing-adsorption recovery of xenon from semiconductor waste gas at approximately 0.1 mole percent xenon concentration, despite the ambient air feedstock being approximately 1,000 times more dilute, because the geographic advantages (zero dehydration, zero blower power, enhanced sorbent capacity due to sub-ambient temperature) effectively compensate for the lower feedstock concentration.

In certain embodiments, the wind-driven ambient capture system, when operated at geographically favorable sites and powered entirely by wind energy, produces xenon with zero direct greenhouse gas emissions and a substantially lower ecological footprint than conventional cryogenic air separation, which requires grid-powered cryogenic refrigeration and processes millions of liters of air through energy-intensive phase changes. This ecological advantage, combined with the energy efficiency described above, supports the economic viability of distributed ambient xenon harvesting at favorable geographic locations as a complement to or substitute for conventional centralized cryogenic production.

### Qualitative Regime Shift and Non-Obviousness of the Disclosed Architecture

The energy economics disclosed herein reflect a qualitative regime shift in the approach to xenon production, not merely an incremental improvement over prior methods. Conventional cryogenic xenon production operates by a “liquefy-everything” paradigm: the entire air stream is cooled to cryogenic temperatures, liquefied, and fractionated, with xenon appearing as a trace co-product of an energy-intensive phase change applied indiscriminately to all atmospheric constituents. The disclosed ambient-capture architecture operates by a fundamentally different paradigm: xenon is selectively extracted from air at or near ambient conditions by exploiting the differential thermodynamic affinity of xenon for appropriately chosen selective media, without requiring any phase change of the bulk air stream.

This paradigm difference has several non-obvious consequences that would not be apparent to a person of ordinary skill in the art accustomed to the cryogenic paradigm. First, the energy scaling with respect to xenon concentration is qualitatively different: in cryogenic production, the energy cost per liter of xenon is dominated by the energy to cool and liquefy the entire air stream and is only weakly dependent on xenon concentration, whereas in the disclosed ambient-capture architecture, the energy cost is dominated by pretreatment (dehydration) and air-handling costs that can be reduced by orders of magnitude through geographic site selection—a degree of freedom that is physically meaningless in the cryogenic paradigm because cryogenic systems already operate at temperatures far below any terrestrial ambient condition. Second, the integration of dehydration elimination through geographic site selection with wind-driven passive airflow and temperature-enhanced sorbent capacity produces a multiplicative energy advantage that is not predictable from analysis of any single subsystem improvement but emerges from the architectural combination. Third, the same platform that operates at the lowest xenon concentrations in ambient air also operates at the highest xenon concentrations in recirculated administration environments, with the same core principles of selective adsorption, fraction-cut enrichment, and thermal-swing regeneration scaled across a dynamic range of approximately six orders of magnitude in feed concentration—a multi-regime adaptability that has no counterpart in the cryogenic approach and that enables the closed-loop capture-administer-recapture-regenerate-reuse cycle that is central to the economic viability of the disclosed platform.

### Regeneration and Release Modes

In certain embodiments, regeneration of a xenon-selective stage is achieved by changing one or more of temperature, pressure, purge composition, electrical state, electromagnetic field, mechanical state, or combinations thereof. Non-limiting examples include temperature swing adsorption, vacuum swing adsorption, pressure swing adsorption, vacuum-temperature swing adsorption, purge-assisted desorption, resistive heating, Joule heating, induction heating, microwave heating, radiofrequency heating, radiant heating, convective heating, fluid-loop heating, field-assisted release, mechanical agitation, or any hybrid sequence thereof. The invention expressly contemplates regeneration that is batch, segmented, partial, full, rapid-cycle, slow-cycle, continuous, or quasi-continuous. In certain embodiments, one or more regeneration temperatures, pressure levels, pressure-ramp profiles, hold times, purge compositions, purge flow rates, electrical inputs, or combinations thereof are selected according to media class, feed regime, humidity burden, impurity burden, target purity, target recovery, contactor geometry, cycle history, or combinations thereof, and may differ materially between ambient-air, recirculated-atmosphere, and waste-stream embodiments.

In some embodiments, a structured contactor is heated directly because the support is conductive or because a conductive network is embedded in or attached to the support. In certain embodiments, the medium is heated indirectly through the shell, a heat exchanger, a circulating thermal fluid, infrared radiation, or a heated purge stream. In certain embodiments, a first reduced pressure is applied before heating to remove free gas and weakly retained non-product gases; a second reduced pressure is applied during or after heating; or distinct pressures are applied to different volumes or different stages at different times. In certain embodiments, regeneration is shared across multiple beds or modules using a common heat source, common vacuum source, or common purge source.

In certain embodiments, regeneration is intentionally incomplete or selectively staged. For example, a first release step may remove primarily residual void gas and weakly bound contaminants; a second release step may produce a xenon-enriched fraction; and a third release step may remove a lower-value tail fraction or one or more late-releasing impurities. This staged approach may be advantageous when seeking to improve product purity, reduce recompression burden, or preserve the utility of a downstream polishing stage.

In certain embodiments, regeneration is assisted by a cryogenic micro-trap or cold trap positioned downstream of the xenon-selective stage and configured to condense or freeze xenon from the desorbed gas stream at a temperature of approximately 120 to 165 Kelvin. In certain embodiments, the micro-trap is operated at a temperature of approximately 130 to 140 Kelvin, which is below xenon's normal boiling point of approximately 165 Kelvin, such that even at the reduced xenon partial pressures encountered during vacuum-assisted desorption, xenon vapor pressure is sufficiently low to drive condensation or desublimation onto the trap surface while nitrogen, oxygen, and argon remain in the gas phase. In certain embodiments, the cryogenic micro-trap is physically compact, having an internal volume of less than approximately 100 milliliters, such that the condensed or frozen xenon is collected in a reduced dead-volume geometry. In certain embodiments, the cryogenic micro-trap is later warmed to release collected xenon at substantially higher purity than the desorbed stream, achieving a polishing function in addition to a collection function. In certain embodiments, the cryogenic micro-trap is cooled by a miniature Stirling cryocooler, pulse-tube cooler, Gifford-McMahon cooler, Joule-Thomson stage, thermoelectric cascade, liquid nitrogen, liquid argon, or other cryogenic source. In certain embodiments, the micro-trap serves as a downstream concentrator that receives desorbed gas from multiple regeneration cycles or from multiple beds, thereby accumulating xenon across several cycles before a single product-release event.

### Multi-Bed Architecture and Cycle Optimization

In certain embodiments, the xenon-selective capture subsystem comprises two, three, four, or more beds or modules arranged for coordinated cycling. The selection of bed count involves a trade-off between working capacity utilization, cycle dead time, capital cost, and system complexity. In certain embodiments, a three-bed architecture is employed in which one bed is in adsorption mode, one bed is in regeneration mode, and one bed is in cooling or standby mode at any given time. This three-bed arrangement offers a favorable balance because each bed may be regenerated at higher temperature and for a longer duration than in a two-bed arrangement of equivalent throughput, thereby achieving more complete xenon release and correspondingly lower residual loading at the start of each adsorption cycle. In certain embodiments, the working capacity required per bed in a three-bed system is approximately 0.40 millimoles per kilogram or less, compared to approximately 0.80 millimoles per kilogram in a two-bed system of the same total throughput, enabling the use of sorbent materials with moderate but practical working capacities under cyclic conditions. In certain embodiments, four or more beds are employed to further reduce the per-bed working-capacity requirement, to provide redundancy, or to enable continuous product flow. In certain embodiments, dead-volume management between beds is coordinated with the multi-bed cycle: a first void-clearing step evacuates or purges residual gas from the connecting manifold and downstream lines before the xenon-rich desorption step begins, such that void gas from the preceding bed does not dilute the product fraction from the current bed.

In certain embodiments, the multi-bed system employs a rotary valve, rotary distribution manifold, or indexed valve assembly configured to route gas to and from each bed according to the cycle phase without requiring independent actuation of each individual bed valve. In certain embodiments, the rotary valve reduces the total dead volume of the manifold and valve network by providing short, direct flow paths between the valve and each bed, reduces the number of individual solenoid or pneumatic valves required, and improves cycle timing repeatability. In certain embodiments, the manifold assembly is configured such that the total internal free-gas volume between the bed outlet and the downstream collection or analyzer stage is less than approximately 10 percent, or less than approximately 5 percent, or less than approximately 2 percent of the internal free-gas volume of the bed itself. In certain embodiments, the pump-down time for the bed-to-collection manifold segment follows the relationship t≈(V/S)×ln(p₀/p₁), where V is the manifold volume, S is the pumping speed, p₀ is the initial pressure, and p₁ is the target pressure, and this pump-down time is configured to be less than approximately 10 percent of the total regeneration interval, such that dead-volume evacuation does not materially reduce the time available for product-fraction collection.

To minimize xenon loss during bed-switching transitions, the gas routing subsystem is configured with a manifold dead volume of less than 50 mL in absolute terms and less than 10% of the total contactor internal volume. Switching valves are positioned within 30 cm of the bed outlet to reduce the volume of gas trapped in connecting conduits during transitions. In embodiments employing a rotary valve, the valve body incorporates sweep ports that flush residual gas from the dead space into either the product stream or the feed stream, depending on the phase of the adsorption cycle, thereby recovering xenon that would otherwise be lost to atmospheric venting.

### Staged Concentration Cascades

In many embodiments, the concentration core includes a staged concentration cascade. A primary collector receives the initial feed and captures or enriches xenon from that feed. A secondary concentrator then receives a xenon-bearing fraction derived from the primary collector and produces a gas or fraction of higher xenon concentration or lower impurity burden. An optional polishing stage may then remove one or more additional impurities, prepare a certified product, or route the gas to blending, storage, delivery, or reuse. The primary collector may be relatively large and configured for throughput; the secondary concentrator may be relatively small and configured for purity or further enrichment; and the polishing stage may be selected according to the requirements of the target application.

In certain embodiments, the output of the primary collector is not sent directly to bulk storage or a user environment, but instead is routed to a secondary micro-bed, a reduced-volume intermediate vessel, a secondary adsorption stage, a membrane stage, a cryogenic stage, or a chromatographic stage. In certain embodiments, multiple primary collectors may feed one secondary concentrator. In certain embodiments, a secondary stage may serve as a temporary xenon buffer and may later desorb to a still smaller polishing or storage stage. In certain embodiments, moderate compression may be applied only after an initial enrichment threshold has been reached.

In certain embodiments, continuous or quasi-continuous xenon recovery is achieved by operating a plurality of xenon-selective modules in staggered adsorption and regeneration states, by using one or more rotating wheels or segmented moving contactors, by using moving-bed or cartridge-exchange architectures, or by combinations thereof. In certain embodiments, two or more modules share heat, vacuum, purge, cooling, or analyzer resources. In certain embodiments, one or more moving or exchanged modules are removed from a capture path and routed to an on-site or off-site regeneration workflow while one or more different modules remain in service.

As used herein, “primary collector” and “primary xenon collector” are used interchangeably to refer to a first-stage collection or enrichment element within a staged concentration cascade.

### Dead-Volume Management and Microvolume Collection

In trace-feed embodiments, product purity and productability may be dominated not only by media selectivity but also by residual void gas present during release and collection. Accordingly, in certain embodiments the concentration core is configured to minimize or manage dead volume throughout the flow path. Non-limiting examples include shortening manifolds, positioning valves and gauges close to the separation stage, minimizing unused shell space, using purgeable or evacuable line segments, using shaped or nested internal supports to reduce nonproductive internal volume, and using small intermediate collection vessels instead of collecting directly into large shells or tanks.

In certain embodiments, a first reduced pressure is applied to a relatively large volume to remove nonproductive gas, while a deeper reduced pressure is applied only to a much smaller downstream volume. This architecture may materially improve xenon mole fraction in the collected cut while avoiding the full energy or hardware burden of pulling a very large vessel to deep vacuum. In certain embodiments, the system may evacuate a primary vessel only to a moderate vacuum, isolate the primary vessel, and then perform deeper vacuum operations on a smaller downstream concentrator or collection volume. In certain embodiments, the system may perform an evacuate-isolate-collect sequence or other valve choreography designed to keep nonproductive residual gas from dominating the final collection fraction.

### Fraction-Cut Collection and Analyzer-Guided Routing

In many embodiments, the system does not treat the desorbed or released gas as a single homogeneous product. Instead, the system may separate the release into one or more fractions based on one or more of elapsed time, temperature, temperature rate, pressure, pressure rate, vacuum depth, flow, composition, detector output, predicted composition, or combinations thereof. A first non-product fraction may correspond primarily to residual void gas and/or weakly retained components. A second fraction may correspond to a xenon-enriched or target-product window. A third fraction may correspond to a tail fraction or a lower-value or impurity-rich fraction. Any one of these fractions may be vented, recycled, repurified, recompressed, routed back to an upstream stage, routed to a secondary stage, or sent to storage.

In certain embodiments, the transition between fractions is determined by a gas analyzer or other detector. In certain embodiments, analyzer-guided routing is based on xenon concentration alone; in other embodiments it is based on xenon concentration together with one or more of oxygen, nitrogen, argon, krypton, carbon dioxide, moisture, total pressure, flow, or detector-derived composite quality score. In certain embodiments, the system may route an early forecut away from product storage until a xenon-enriched peak is detected or predicted, and may route a later fraction to repolishing or discard after the peak subsides.

In certain embodiments, the enrichment achievable by fraction-cut collection during thermal regeneration depends on the ratio of isosteric heats of adsorption between xenon and the principal competing species (nitrogen, oxygen, argon). For silver-exchanged zeolites, the isosteric heat of xenon adsorption is typically in the range of approximately 35 to 60 kJ per mole, whereas the isosteric heat for nitrogen is typically in the range of approximately 14 to 20 kJ per mole. This differential of approximately 20 to 40 kJ per mole causes xenon to desorb at a different rate and at a different point in the thermal ramp than nitrogen and oxygen, thereby enabling time-resolved separation of a xenon-enriched product fraction from a forecut rich in weakly adsorbed species and void gas. In certain embodiments operating on true ambient air at approximately 87 parts per billion, the molar ratio of void gas to adsorbed xenon may exceed approximately 10,000:1, such that the forecut volume is orders of magnitude larger than the product cut. Under such conditions, fraction-cut collection may achieve enrichment factors of approximately 10 to 100 relative to single-fraction (homogeneous) desorbed gas collection, with the enrichment advantage being largest at the lowest feed concentrations and decreasing as feed xenon concentration increases toward the Langmuir saturation regime. In certain embodiments, two or more sequential fraction-cut stages may be cascaded to achieve additional enrichment, for example from approximately 100 parts per million to approximately 1 to 5 percent xenon by volume in two stages.

### Polishing, Purification, and Output States

After one or more concentration stages, the xenon-bearing fraction may be polished or otherwise further processed. In certain embodiments, polishing may remove one or more of oxygen, nitrogen, argon, krypton, carbon dioxide, water, volatile contaminants, radon, reactive gases, or combinations thereof. Polishing may comprise a secondary adsorption stage, a membrane stage, a microporous crystalline membrane stage, a cryogenic stage, a cold trap, a micro-cryotrap, a chromatographic stage, a carbon-dioxide-sieving stage, an oxidation or reduction stage, or combinations thereof.

The output state of the concentration core may vary by embodiment and by intended use. In certain embodiments, the output is substantially pure xenon. In certain embodiments, the output is a xenon-enriched gas that is not yet pure but is suitable for further storage, blending, certification, or secondary purification. In certain embodiments, the output is a certified xenon product ready for oxygen blending or direct delivery. In certain embodiments, the system is optimized for therapeutic or wellness use and therefore need not achieve semiconductor-grade or ultra-high-purity xenon, but instead outputs a xenon-bearing fraction suitable for preparation of a non-hypoxic xenon-containing mixture. In other embodiments, the system is optimized for scientific, industrial, or imaging applications and therefore includes additional downstream purification.

### Multi-Bed, Continuous, and Intensified Cycle Embodiments

In certain embodiments, the concentration core includes two or more separation modules operated in coordinated fashion such that at least one module is in adsorption mode while at least one different module is in regeneration, cooling, standby, or collection mode. This enables quasi-continuous or continuous operation and may increase average recovery, throughput, or energy efficiency. In certain embodiments, two or more modules share a common vacuum source, heat source, cooling loop, analyzer, or storage manifold. In certain embodiments, two or more modules are operated with staggered timing such that a downstream concentrator or product tank receives a more continuous flow of xenon-bearing gas.

In certain embodiments, one or more separation stages comprise rapid-cycle structured contactors with intensified regeneration. For example, a foil, laminate, monolith, or heat-exchanger-like body carrying xenon-selective media may be electrically heated and rapidly cooled to shorten cycle times and improve bed utilization. In certain embodiments, a rotating wheel or segmented contactor passes through an adsorption zone and a regeneration zone in cyclic manner. In certain embodiments, a multi-bed system uses a large front-end collector together with a rapidly cycling back-end concentrator.

### Facility Recapture Infrastructure

In certain embodiments, the invention comprises facility recapture infrastructure configured to recover xenon from one or more facility capture paths rather than, or in addition to, recovering xenon from true ambient outdoor air. Such facility recapture infrastructure may be deployed in hospitals, surgical centers, dental practices, veterinary clinics, outpatient clinics, medical offices, recovery units, wellness facilities, laboratories, greenhouses, growth rooms, hatcheries, produce-storage facilities, food-distribution facilities, livestock or companion-animal environments, transport environments, industrial environments, residential environments, or other xenon-enabled spaces.

In certain embodiments, xenon recovery from a facility capture path may be adsorption-based, membrane-based, cryogenic, condensation-based, freezing-based, or hybrid, and a given facility may use different recovery modes for different capture paths or different purity targets. Thus, a local scavenging path, a room recirculation path, a return-air path, and an exhaust path may each be served by different xenon-selective recovery hardware while still participating in one common regeneration, quality-assurance, storage, or reuse architecture.

A facility capture path may begin at or near any location where xenon-containing gas is likely to be present in materially elevated concentration relative to the local background. Non-limiting examples include a waste anesthetic gas scavenging interface, an anesthesia machine exhaust or disposal line, a ventilator exhaust line, a breathing-circuit-adjacent pathway, a local hood, a dental face-zone suction path, a procedure-room or recovery-bay recirculation device, a room air purifier, a return grille, a branch return duct, a nonrecirculating exhaust duct, a mechanical-room air handler or skid, a greenhouse recirculation device, a produce-storage recirculation device, a storage-room air handler, a refrigerated-container recirculation path, a truck-trailer air path, a shipboard-container return path, a livestock-enclosure exhaust path, a veterinary holding-room recirculation path, or a session-end purge pathway from a xenon-enabled room, booth, pod, canopy, bay, cabin, or treatment environment.

In certain embodiments, the facility recapture infrastructure comprises one or more modules configured to integrate with an anesthetic gas scavenging system, a waste anesthetic gas disposal pathway, a ventilator exhaust path, or another clinically controlled waste-gas pathway. In such embodiments, a xenon-containing waste stream may be routed through a xenon capture module positioned in-line, side-stream, parallel, bypass, or cartridge form. In certain embodiments, the xenon capture module comprises a dual-layer or multi-layer structure. A first layer may be configured to reduce carbon dioxide, water, or both. A second layer may be configured to selectively capture xenon from the remaining gas stream.

In certain embodiments, xenon is delivered through a sealed or substantially sealed interface, including an anesthesia mask, nasal hood, nasal cannula, ventilator circuit interface, helmet, or other patient interface, and xenon leaked or vented from that interface is recovered from the surrounding room or facility environment via one or more local or delayed environmental recapture paths. Such embodiments may use a room recirculator, air purifier, return-air path, return-duct path, exhaust path, or combinations thereof, and may route recovered xenon to regeneration, storage, blending, certification, or reuse.

In certain embodiments, xenon is recovered from procedure-room exhaust, recovery-bay air, post-anesthesia care unit pathways, critical-care rooms, or other room-scale or bay-scale facility pathways. In some facilities, xenon-bearing gas may be routed toward an exhaust point or mechanical-room discharge location. In such embodiments, a facility capture module may be positioned in or adjacent to the exhaust path, such as at a branch duct, terminal duct, discharge plenum, rooftop housing, mechanical-room skid, or another accessible portion of the exhaust chain. In certain embodiments, a controller interlocked with facility ventilation controls is configured to prevent recirculation of xenon-contaminated or incompletely processed gas except through an authorized xenon recovery path, purge path, exhaust path, or combinations thereof.

In certain embodiments, the facility recapture infrastructure is integrated with one or more HVAC return pathways, return grilles, recirculation loops, fan-coil systems, dedicated room air handlers, or mechanical-room air processing systems. In such embodiments, the invention preferably targets xenon-enabled spaces in which xenon has been intentionally introduced or is expected to be present above ordinary background levels. In certain embodiments, a xenon capture module is configured as a return-grille insert, a return-duct cartridge, a bypass side-stream module, an in-duct structured contactor, a replaceable filter cassette, a purifier-integrated module, or a permanently installed bed.

In certain embodiments, xenon recovery hardware for a facility capture path is embodied as an article configured for installation in an airflow path of a return grille, return duct, room recirculator, air purifier, HVAC return module, canopy return, booth return, or other recirculation or exhaust path. Such an article may comprise a frame, cassette, insert, cartridge, monolith, or other housing carrying one or more xenon-selective media and optionally one or more pretreatment regions configured to reduce water, carbon dioxide, particulates, or combinations thereof upstream of a xenon-selective region. In certain embodiments, the article includes a visible saturation indicator, a machine-readable state element, a compatibility-controlled interface, or combinations thereof, and is removable, replaceable, serviceable, regenerable, or permanently installed. In certain embodiments, the article includes a fluid coupling, docking geometry, keyed feature, latch feature, service geometry, or combinations thereof to facilitate regeneration, replacement, extraction, or servicing.

In certain embodiments, the facility recapture infrastructure comprises one or more room recirculators, air purifiers, portable purifiers, tower units, wall-mounted units, ceiling-mounted recirculators, or similar air-processing devices modified to include a xenon-selective capture stage. In certain embodiments, the purifier or recirculator performs a dual function: maintaining air quality in a clinic, room, office, operatory, bay, waiting area, laboratory, wellness area, residence, greenhouse area, produce-storage room, refrigerated trailer, shipboard container, kennel, stall, pen, coop, or other environment while also capturing xenon that has entered that local environment during or after an intentional xenon-use session.

### Comfort-First Administration Environments

In certain embodiments, the invention comprises one or more comfort-first xenon administration environments configured to deliver xenon to a user in a manner that is more comfortable, less claustrophobic, lower-resistance, and more pressure-neutral than many conventional closed or tightly sealed gas-delivery systems, while still enabling economically meaningful local recapture and/or delayed local environmental recapture.

In many embodiments, the administration environment is semi-open rather than fully closed. A semi-open environment may define a controlled breathing zone without requiring a tight face seal, without requiring a fully closed recirculating breathing loop, and without requiring the user to breathe against substantial resistance. At the same time, the environment may include one or more delivery paths, one or more local capture paths, one or more environmental recapture paths, and one or more safety/interlock paths.

In certain embodiments, the administration environment defines a controlled breathing zone adjacent to, surrounding, directed toward, or otherwise influencing the user's nose and/or mouth. The breathing zone may be established by one or more of directed gas plumes, low-velocity diffusers, canopies, hoods, headspaces, partial enclosures, collars, circumoral frames, headrests, local air curtains, local suction boundaries, perimeter returns, or combinations thereof.

In many embodiments, xenon administration is paired with two layers of recapture: local capture, in which xenon leakage or exhaled xenon is captured near the user or near the intended breathing zone; and delayed local environmental recapture, in which xenon remaining in a pod, booth, room, bay, cabin, or other xenon-enabled space is later or concurrently recovered through recirculation, purifier loops, return paths, exhaust paths, or combinations thereof.

In certain embodiments, the administration environment is configured as a transparent or semi-transparent canopy, pod, recliner module, lounger, bedside enclosure, or partial headspace structure. The use of transparent or semi-transparent walls, panels, or shields may reduce perceived claustrophobia while still defining a controllable gaseous headspace. Such structures may be open-bottom, open-front, side-open, partially enclosed, retractable, or otherwise configured to permit rapid ingress and egress without requiring a sealed face mask or tube.

In certain embodiments, the controlled breathing zone is created or stabilized using one or more air curtains, directed clean-gas sheets, curtain-like jets, rear plenums, headspace shaping flows, or other flow-field management structures that form an aerodynamic rather than physical boundary. In such embodiments, the user may experience a virtual mask or virtual breathing zone formed by airflow rather than by contact hardware.

In certain embodiments, the administration environment is configured for dental, clinic, office-based, chair-side, or minor-procedure settings in which user comfort and staff access are both important. In such embodiments, the system may define a controlled breathing zone around the patient's nose and/or mouth while leaving the treatment field sufficiently accessible for a clinician. In certain embodiments, the delivery and capture geometry preserves direct clinician line-of-sight to the oral treatment field and permits instrument access to the oral treatment field while xenon delivery and xenon capture continue. In certain embodiments, a first capture path is positioned near the patient or treatment field and a second capture path is positioned in the room or recirculation environment.

In certain embodiments, the administration environment is adapted for recovery bays, bedside care, ambulances, vehicles, transport units, home use, office use, or other settings where user comfort, rapid setup, and low-resistance breathing are particularly important. In such embodiments, a canopy, over-bed headspace, seat-adjacent plenum, vehicle headspace, or localized room corner system may be used to define the controlled breathing zone.

In certain embodiments, the administration environment is configured as a single-user booth, multi-user lounge, spa recliner environment, shared room with individualized breathing zones, treatment alcove, wellness pod, or related semi-open environment. In such embodiments, xenon may be delivered in a manner intended to feel more like a comfortable space or ambient headspace than a medical mask or breathing circuit.

In certain embodiments, the administration environment includes one or more monitoring and safety subsystems configured to verify that user breathing conditions and room/environment conditions remain within selected bounds. Monitored variables may include xenon concentration in one or more local zones, oxygen concentration, carbon dioxide concentration, pressure, flow, occupancy, user presence, capture-path status, valve state, cartridge state, ventilation state, or combinations thereof. In many embodiments, the system includes oxygen assurance logic such that xenon delivery is limited, interrupted, or modified unless a selected minimum oxygen condition is met.

In certain embodiments, the administration environment is adapted for veterinary palliative care, hospice care, comfort-centered terminal care, or other legally authorized end-of-life settings in which reduced distress, low-resistance breathing, caregiver access, monitoring, and a calm surrounding environment are important. In such embodiments, the disclosed systems are not limited to curative, performance, or recovery-oriented use.

In certain embodiments, semi-open xenon administration uses local capture adjacent the controlled breathing zone without a delayed local environmental recapture subsystem. In other embodiments, semi-open xenon administration uses delayed local environmental recapture from a surrounding space without a dedicated local capture subsystem adjacent the controlled breathing zone. In still other embodiments, both local capture and delayed local environmental recapture are used together.

In certain embodiments, a semi-open xenon administration environment provides the controlled breathing zone and safety architecture irrespective of whether xenon-selective recapture is contemporaneous, delayed, partial, shared, or absent from the immediate administration apparatus, such that the delivery environment may be practiced independently of a particular recapture economics or backend recovery workflow.

The xenon-oxygen mixture density provides a physical containment advantage in semi-open administration embodiments. For a 50/50 molar mixture of xenon and oxygen at body temperature of approximately 310 K, the mixture density is approximately 2.7 to 3.0 times the density of room air at approximately 298 K, calculated from ideal-gas relations using the mixture molar mass of approximately 81.5 grams per mole compared to approximately 29 grams per mole for air. This density contrast causes the xenon-enriched mixture to settle and pool within the controlled breathing zone in a manner analogous to a dense liquid in a basin. The Richardson number—a dimensionless ratio of buoyancy forces to shear forces—exceeds approximately 100 under typical room conditions with gentle air circulation at velocities below approximately 0.5 meters per second, indicating strongly buoyancy-dominated stratification. Under such conditions, turbulent mixing between the dense breathing-zone gas and the lighter room air is substantially suppressed, and the gas-curtain flow required to maintain target xenon concentration may be reduced by a factor of approximately 2 to 4 compared to a design that does not account for buoyancy. In certain embodiments, the gas-curtain slot is positioned and oriented to reinforce the natural density stratification, and the total curtain flow rate is selected in the range of approximately 15 to 25 cubic feet per minute for a breathing zone of approximately 35 to 50 liters, which is substantially lower than the approximately 40 to 70 cubic feet per minute that would be required for a neutrally buoyant delivery gas.

The buoyancy-assisted containment described above enables rich-loop recirculation architectures in which xenon losses from the breathing zone are sufficiently low that continuous xenon-selective adsorption of the full loop gas is unnecessary. In certain embodiments employing rich-loop recirculation, the primary gas loop maintains a xenon-enriched atmosphere in the controlled breathing zone and routes the loop gas through a carbon dioxide scrubber and an oxygen replenishment stage without passing the bulk loop gas through a xenon-selective adsorption stage. In such embodiments, the primary xenon loss mechanism is leakage from the breathing zone boundary, which is substantially mitigated by the buoyancy-assisted containment described herein. Based on first-principles mass balance analysis, the net xenon consumption per session under rich-loop recirculation may be in the range of approximately 2 to 10 liters per session, depending on breathing-zone geometry, curtain design, session duration, and room recovery efficiency, compared to approximately 10 to 20 liters per session for an architecture relying on dilute-recapture from room air. In certain embodiments, leaked xenon is recovered from the room environment via one or more delayed environmental recapture modules, with recovery efficiencies of approximately 60 to 90 percent, further reducing net xenon consumption. In certain embodiments, the combination of rich-loop recirculation and environmental recapture enables a “xenon-neutral” operating mode in which the ambient harvesting rate from a continuous-operation xenon capture module approximately equals or exceeds the net xenon loss rate across a day's administration sessions, such that the facility's xenon inventory is maintained or grows over time without requiring external xenon purchases beyond initial inventory.

In embodiments where the semi-open administration apparatus operates within a mechanically ventilated space, the system may include a ventilation-interlock mode in which the controller communicates with building HVAC controls to reduce or redirect supply-air flow within the breathing zone during xenon delivery. The local-capture flow rate is configured to exceed cross-draft velocity by at least a factor of two (≥2×), ensuring that exhaled xenon-enriched gas is entrained before room currents can dilute it. In certain embodiments, draft-management elements—such as partial enclosures, deflector panels, or air curtains operating at 0.15 to 0.25 m/s diffuser velocity—may surround the breathing zone to attenuate lateral airflow without creating a sealed enclosure. This ventilation-interlock architecture allows the semi-open administration apparatus to maintain high recapture efficiency (≥85%) even in clinical or commercial spaces with active HVAC systems.

### Active Atmosphere-Concentration Management in Xenon-Enabled Spaces

In certain embodiments, a xenon-enabled space includes both a xenon delivery subsystem and a xenon recovery subsystem operated under feedback control to maintain xenon concentration in a selected range within at least a portion of the space. One or more xenon sensors, oxygen sensors, carbon dioxide sensors, occupancy sensors, or combinations thereof may be used to modulate xenon addition, xenon removal, purge operation, room recirculation, and delivery timing. In certain embodiments, xenon addition, xenon removal, target xenon concentration, target xenon concentration range, target xenon concentration trajectory, delivery timing, session duration, or combinations thereof are modulated based at least in part on one or more user states associated with one or more users in the xenon-enabled space. Non-limiting examples of such user-state inputs include electroencephalography (EEG) signals or derived EEG features, oxygen-saturation state, respiratory rate or respiratory pattern, heart-rate state, heart-rate-variability state, motion or activity state, posture, blood-pressure state, perfusion state, temperature state, skin-conductance state, arousal state, sedation state, level-of-consciousness state, sleep state, relaxation state, clinician-entered target state, supervisory input, or combinations thereof. In certain embodiments, control logic is based on an individual user state, an aggregate state across multiple users, a most conservative user state, a weighted combination of user states, or a supervisory override.

In certain embodiments, the xenon-enabled space comprises a plurality of user-associated subzones or localized breathing zones, and xenon addition, xenon removal, or both are modulated on a subzone-by-subzone basis based on the corresponding user state or user states while a broader room-level xenon state is separately maintained, limited, or supervised. In one non-limiting class of embodiments, a user seated in a chair, recliner, booth, bay, or other xenon-enabled space is monitored using a forehead-mounted EEG sensor or other user-state sensor while the controller titrates xenon concentration, target range, target trajectory, session duration, or combinations thereof toward a desired therapeutic, relaxation, meditation, recovery, or supervisory target state. In certain embodiments, xenon concentration or xenon state within the xenon-enabled space is determined directly by one or more xenon sensors, indirectly by one or more xenon-state estimators, or by combinations thereof. In certain embodiments, the system functions as a xenon atmosphere-conditioning appliance configured to increase, decrease, hold, or re-establish a target xenon concentration while maintaining a selected minimum oxygen condition and while routing recovered xenon to regeneration, storage, blending, certification, or reuse. In certain embodiments, the xenon recovery subsystem includes a room recirculator, air purifier, return-air module, HVAC return module, local canopy return, booth return, or combinations thereof.

In certain embodiments, the xenon atmosphere-conditioning appliance or associated controller is configured to receive one or more remote setpoints, remote monitoring inputs, remote authorization inputs, maintenance-state inputs, service-state inputs, or combinations thereof over a wired or wireless network connection. In certain embodiments, such inputs are used to permit, restrict, enable, disable, or otherwise modify xenon addition, xenon removal, purge operation, regeneration scheduling, compatibility-controlled release, or combinations thereof. In certain embodiments, cumulative xenon delivered, cumulative xenon recovered, regeneration history, cartridge or module state, and quality-assurance state are recorded locally, remotely, or both. In certain embodiments, operation may be restricted when a selected service, compatibility, maintenance, or authorization condition is not satisfied.

In certain embodiments, the xenon atmosphere-conditioning system is embodied as a standalone appliance comprising a housing, a gas-moving subsystem configured to circulate air from a xenon-enabled space through the housing, a xenon delivery subsystem configured to add xenon to the xenon-enabled space, and a xenon recovery subsystem disposed within or fluidly coupled to the housing and configured to remove xenon from circulated air. In certain embodiments, the appliance is freestanding, wall-mounted, ceiling-mounted, integrated into a room recirculator or air purifier, or combinations thereof. In certain embodiments, the appliance is configured for clinic, wellness, recovery-bay, home, office, vehicle, or transport use.

In certain embodiments, a xenon atmosphere-management system is room-integrated and distributed, with xenon delivery outlets, recapture inlets, sensors, and control elements disposed at different locations in or adjacent to the xenon-enabled space rather than within a single housing. In certain embodiments, such distributed elements are positioned in ceilings, walls, canopies, booths, return plenums, headrests, seat-adjacent structures, HVAC return structures, or combinations thereof.

### Biological Conditioning Environments for Plants, Animals, Produce, and Other Biological Materials

In certain embodiments, xenon is introduced into or recaptured from a biological-conditioning environment containing one or more non-human biological targets, including without limitation plant material, harvested produce, seeds, seedlings, eggs, embryos, tissues, organs, cultures, livestock, companion animals, poultry, aquaculture species, or combinations thereof. Such environments may be operated at ambient pressure, near-ambient pressure, or other non-pressurized conditions and may include growth chambers, greenhouse zones, incubators, kennels, stalls, pens, coops, hatcheries, storage rooms, pallet enclosures, refrigerated trailers, shipboard containers, warehouse modules, or similar environments.

In certain embodiments, a xenon-containing atmosphere or locally xenon-enriched recirculation loop is used for preservation, storage, transport, cultivation, stress mitigation, recovery, or other biological handling objectives. In certain embodiments, the environment includes one or more recirculation paths, return paths, purifier paths, or exhaust paths carrying xenon to one or more xenon recovery modules or xenon recovery inserts during or after a conditioning interval. In certain embodiments, control is coordinated with temperature, humidity, oxygen concentration, carbon dioxide concentration, ethylene concentration, occupancy state, ripening state, spoilage state, or combinations thereof.

In one non-limiting class of embodiments, harvested produce or other plant material is transported or stored within a refrigerated room, pallet enclosure, truck trailer, ship hold, or intermodal container while a xenon delivery subsystem establishes or replenishes a xenon-containing atmosphere and a recirculation subsystem routes withdrawn gas through one or more xenon recovery modules for delayed local environmental recapture, regeneration, and reuse.

In another non-limiting class of embodiments, a veterinary holding environment, livestock compartment, animal transport bay, recovery enclosure, or similar environment is provided with xenon through a local breathing zone, enclosure headspace, or conditioned recirculation flow while oxygen assurance, purge capability, local capture, delayed local environmental recapture, or combinations thereof are maintained.

### Personal, Wearable, Portable, and Distributed Body-Worn Xenon Systems

In certain embodiments, the disclosure provides a distinct category of personal xenon devices configured for use by or on a single user and not dependent on conventional anesthesia-machine architecture, bulky closed breathing circuits, or room-scale installations. Such devices may be configured for home use, sleep or relaxation support, recovery, travel, outdoor or expedition use, social or hospitality settings, retreat settings, supervised clinical use outside traditional anesthesia-machine architecture, or combinations thereof. In certain embodiments, the same architectural family is scalable from low-dose enrichment embodiments to more tightly controlled wellness embodiments and to supervised higher-acuity embodiments using analogous delivery, capture, monitoring, and reuse logic.

In certain embodiments, the personal xenon device creates a personal breathing microenvironment adjacent to the user's nose and/or mouth without requiring a fully closed recirculating breathing loop, without requiring a tight face seal, and without requiring saturation of an entire room. The localized microenvironment may be established by a nasal interface, nasal prongs, a nostril interface, a nostril insert, a nasal clip, a cannula, a circumoral interface, a partial face structure, a transparent lower-face shell, a collar-like boundary, a gaiter-integrated interface, a scarf-or wrap-integrated interface, a textile-integrated interface, a pendant-adjacent diffuser, a headrest-adjacent structure, or combinations thereof.

In certain embodiments, exhaled or leaked xenon is intercepted before full ambient dilution by a local capture subsystem positioned at or adjacent to a lower-face region, neck region, collar region, chest region, shoulder region, textile region, or another personal microenvironment region of the user. The local capture subsystem may comprise one or more inlets, including a collar inlet, a necklace-like capture ring, a gaiter-integrated inlet, a shoulder-mounted inlet, rings, slots, plumes, capture boundaries, pendant structures, chest pods, textile channels, or exhalation receivers. In certain embodiments, the system passively captures naturally exhaled gas. In certain embodiments, the system additionally or alternatively receives user-directed exhalation into an exhalation receiver when higher recovery efficiency is desired.

In certain embodiments, the personal xenon device employs a distributed body-worn architecture in which lightweight face-adjacent delivery or sensing elements are separated from heavier elements including one or more source vessels, xenon recovery modules, pumps, valves, batteries, control electronics, regeneration subsystems, dehumidification subsystems, or combinations thereof. Such heavier elements may be positioned in a collar, pendant, chest pod, vest, jacket, harness, belt pack, backpack, or other user-carried structure. In certain embodiments, the personal delivery interface and the local capture subsystem are configured to preserve visibility of at least a portion of the user's lower face and to permit speech, ambulation, work, relaxation, social interaction, or combinations thereof during xenon delivery. In certain embodiments, the personal delivery interface and the local capture subsystem are configured to permit visual social interaction while xenon delivery continues. In certain embodiments, the resulting interface is less stigmatizing, less claustrophobic, and more compatible with non-clinical or mixed-use settings than conventional closed masks or tightly sealed breathing hardware, thereby improving adherence, discretion, and broader deployability.

### Personal Moisture Management, Portable Regeneration, and Humidity Reuse

In certain personal, wearable, or portable embodiments, gas captured from the personal breathing microenvironment is routed first through a moisture-management subsystem before xenon-selective recovery. The moisture-management subsystem may reduce water vapor by condensation, sorption, membrane separation, dehumidification wheels, desiccants, or combinations thereof. In certain embodiments, water removed from captured gas is stored, condensed, sorbed, or otherwise recovered and later routed to a humidification subsystem configured to humidify gas delivered back to the user, thereby improving comfort and reducing dryness while reducing external water demand.

In certain embodiments, xenon recovered from the captured gas is retained in one or more xenon-selective media and later released by a portable or dockable regeneration subsystem. The regeneration subsystem may use heating, pressure reduction, mild vacuum, or combinations thereof and may release xenon into a reduced-volume collection region to reduce dead-volume dilution. In certain embodiments, released xenon is reused directly, blended with oxygen or air, stored temporarily, or routed to a dock, cradle, station, or service workflow for later handling. In certain embodiments, one or more personal subsystems, including an exhalation receiver, pendant, chest pod, collar unit, belt-carried subsystem, xenon recovery module, or combinations thereof, are configured to dock with a cradle, charger, bedside unit, refill unit, service station, or regeneration station for charging, regeneration, refill, data exchange, synchronization, software update, or combinations thereof.

### Cartridge, Docking, Regeneration, Quality-Assurance, and Logistics Ecosystem

In certain embodiments, the invention comprises an integrated cartridge, module, docking, regeneration, quality-assurance, blending, storage, and logistics ecosystem configured to support xenon capture, xenon recapture, xenon concentration, xenon reuse, or combinations thereof across multiple feed regimes and multiple deployment environments.

In certain embodiments, a xenon capture element is embodied as a removable cartridge, replaceable cassette, reloadable vessel, slide-in drawer, rigid canister, purifier module, duct module, room module, pod module, scavenging module, wearable module, headspace module, or permanently installed contactor. The invention expressly includes both removable and fixed forms. The same regeneration, quality-assurance, certification, release-gating, and compatibility-control logic may apply to removable, replaceable, reloadable, semi-permanent, and permanently installed xenon recovery modules.

In certain embodiments, a module, vessel, or xenon-bearing batch progresses through one or more chain-of-custody states selected from loaded, awaiting regeneration, in regeneration, awaiting certification, certified, awaiting blending, dispensed, returned to service, quarantined, downgraded, retired, or combinations thereof. Such states may be embodied physically, electronically, optically, mechanically, logically, or in hybrid form, and may be updated at one or more handoffs, docking events, regeneration events, analysis events, release events, or combinations thereof. In certain embodiments, the chain-of-custody state is tracked locally on the module or vessel. In certain embodiments, it is tracked in a local or distributed record system.

In certain embodiments, regenerated modules are returned to service separately from recovered xenon-bearing product. For example, a regeneration center may return regenerated modules to a capture site while routing certified xenon-bearing product to storage, blending, a different facility, or a different end user. In certain embodiments, the same quality-assurance, certification, release-gating, and compatibility-control logic applies whether the xenon recovery module is removable, replaceable, reloadable, or permanently installed. In certain embodiments, one workflow is used for module return and a different workflow is used for xenon-product release, thereby allowing the ecosystem to function across on-site, off-site, centralized, distributed, single-actor, and multi-actor operating models.

In certain embodiments, each module or cartridge includes one or more identity elements and one or more state indicators. A state indicator may include one or more of a colorimetric indicator, electronic memory, RFID or NFC tag, optical code, mechanical index, pressure-drop signal, mass-change signal, downstream xenon signal, embedded temperature or pressure sensor, flow history, regeneration count, time-in-service counter, or combinations thereof. A module's state may include one or more of saturation level, last regeneration date, number of regeneration cycles, source-stream class, intended use class, impurity exposure history, quality verification status, and compatibility class.

In certain embodiments, the ecosystem comprises one or more docking or regeneration stations configured to receive one or more modules, connect physically and/or fluidically to those modules, regenerate xenon-selective media therein, recover one or more xenon-bearing fractions, cool or recondition the modules, and prepare the modules for return to service, storage, or retirement. A docking or regeneration station may be configured for single-module operation, multi-module batch operation, rack-based operation, carousel-based operation, drawer-based operation, conveyor-based operation, or combinations thereof.

In certain embodiments, regeneration is performed on-site at the point of capture. In certain embodiments, regeneration is performed off-site by a separate service provider. In certain embodiments, capture is decentralized but regeneration is centralized. In certain embodiments, modules are transported through a controlled logistics loop to and from a regeneration center. In certain embodiments, the regeneration center returns regenerated modules and separately returns certified xenon-bearing product, whereas in other embodiments the regeneration center returns regenerated modules only and retains or separately distributes the recovered xenon product.

In certain embodiments, the ecosystem includes a quality-assurance and certification subsystem configured to measure, infer, verify, or record one or more quality attributes of a recovered xenon-bearing fraction or product. Such quality attributes may include xenon concentration, oxygen concentration, carbon dioxide concentration, moisture content, pressure, source class, impurity class, product class, intended use class, regeneration history, leakage status, or combinations thereof. The measured or inferred result may be stored as a printed certificate, electronic certificate, machine-readable authorization, physical tag, logical release token, or combinations thereof.

In certain embodiments, one or more interfaces in the ecosystem are keyed, indexed, coded, or otherwise restricted according to concentration class, certification class, intended use class, module type, or compatibility class. This keyed or coded behavior may be mechanical, dimensional, magnetic, optical, electronic, logical, fluidic, or combinations thereof. In certain embodiments, a capture module physically cannot dock with an incompatible regeneration station. In certain embodiments, a regenerated product vessel cannot be dispensed through an incompatible downstream interface.

In certain embodiments, xenon recovered from one or more modules is routed to an intermediate vessel, buffer vessel, storage cylinder, tank, distribution line, blending manifold, local delivery device, or another downstream product state. In certain embodiments, recovered xenon is stored as a xenon-rich gas pending later polishing or blending. In certain embodiments, it is immediately blended with oxygen, air, or another gas to form a non-hypoxic xenon-containing mixture. In certain embodiments, it is routed to a secondary concentration or polishing stage before final certification.

In certain embodiments, xenon recovered from a local capture path, delayed local environmental recapture path, or facility gas path is not purified to the highest available purity class before reuse. Instead, the recovered xenon-bearing gas may be assigned to a local reuse class and reused within the same environment, the same site, or a different xenon-enabled space of the same site when one or more selected oxygen, concentration, impurity, or certification conditions are satisfied. In other embodiments, the same recovered xenon-bearing gas is routed to additional purification or concentration stages before reassignment to a higher product class.

In certain embodiments, a module, vessel, interface, or appliance carries one or more external indicia corresponding to a concentration class, intended use class, compatibility class, certification state, service state, or combinations thereof. Such external indicia may be visual, geometric, tactile, optical, machine-readable, or combinations thereof. In certain embodiments, the external indicia cooperate with a keyed or compatibility-controlled interface. In certain embodiments, the external indicia allow a user, inspector, service provider, regulator, or counterparty to determine a class or state of the module, vessel, or appliance without accessing internal data structures.

### Central Site Xenon Utility and Exchange-Vessel Workflows

In certain embodiments, a site includes a plurality of xenon-enabled spaces together with a central or semi-central xenon utility subsystem configured to receive xenon from one or more local capture paths, delayed local environmental recapture paths, facility gas paths, or combinations thereof, and to redistribute xenon-containing gas to one or more xenon-enabled spaces.

In certain embodiments, xenon-bearing gas is stored, transported, blended, dispensed, or exchanged in a xenon-bearing gas vessel comprising a pressure vessel, cylinder, bottle, cartridge, tank, or other container having an outlet interface with a compatibility-controlled geometry or other compatibility-controlled feature. In certain embodiments, the vessel further includes at least one external indicium corresponding to a concentration class, intended-use class, certification state, local reuse class, service state, or combinations thereof. Such an external indicium may be visual, geometric, tactile, optical, machine-readable, or combinations thereof, and may allow a user, service provider, regulator, or automated system to determine a class or state of the vessel without accessing internal data structures. In certain embodiments, the external indicium cooperates with a keyed, indexed, coded, or otherwise compatibility-controlled interface.

In certain embodiments, the central or semi-central xenon utility subsystem comprises one or more regeneration stations, concentration stages, quality-assurance or certification stages, source vessels, source manifolds, blending manifolds, exchange-vessel interfaces, or combinations thereof.

In certain embodiments, a first set of vessels, cartridges, or modules contains xenon-bearing gas or loaded capture media returned from one or more xenon-enabled spaces, and a second set of vessels, cartridges, or modules contains regenerated, certified, blended, or otherwise prepared xenon product routed back to one or more xenon-enabled spaces.

In certain embodiments, xenon recovered from a local capture path, a delayed local environmental recapture path, or another facility gas path is assigned to a local reuse class and is reused within the same xenon-enabled space, within a different xenon-enabled space of the same site, or within another site of a service network when one or more selected oxygen, concentration, impurity, certification, or intended-use conditions are satisfied. In other embodiments, the same recovered xenon-bearing gas is routed to further concentration, purification, polishing, certification, or combinations thereof before assignment to a higher product class. In certain embodiments, a local reuse class corresponds to a physical destination selected from a local reuse vessel, a local reuse manifold branch, a local reuse dispenser, a local reuse appliance inlet, or combinations thereof.

In certain embodiments, redistribution occurs within the same site. In certain embodiments, redistribution occurs across multiple sites in a service network. In certain embodiments, xenon recovered from one space is reused in the same space, a different space in the same facility, or a different facility, depending on quality class, intended use class, certification state, or combinations thereof.

In certain embodiments, a xenon recovery module or other xenon capture article is single-use, replaceable, refreshable, retired, or otherwise processed after loading, and xenon is recovered from that article by thermal, vacuum, cryogenic, chemical, dissolution, or hybrid extraction rather than by immediate in situ regeneration. The article may then be discarded, recycled, reprocessed, or returned to service.

### Monitoring, Safety, Control Logic, and Eligibility Gating

In certain embodiments, the concentration core, facility recapture infrastructure, administration environments, and cartridge ecosystem are controlled using one or more measured, inferred, or predicted physical variables, including without limitation temperature, pressure, reduced pressure, pressure rate of change, humidity, carbon dioxide concentration, oxygen concentration, xenon concentration, saturation state, breakthrough state, valve state, flow, differential pressure, adsorbent age, regeneration history, power availability, storage capacity, safety state, demand forecast, or combinations thereof. In administration embodiments, one or more control actions may additionally be based on one or more user states associated with one or more users. Such user states may be measured directly, inferred indirectly, predicted, or received from a wearable, bedside, chair-mounted, canopy-mounted, or remote monitoring device and may include physiological, neurological, electrophysiological, biometric, behavioral, positional, activity-related, or supervisory states. In certain embodiments, the resulting control logic increases, decreases, holds, tapers, ramps, or otherwise modulates xenon concentration, target range, target trajectory, xenon delivery timing, session duration, purge criteria, recapture timing, or combinations thereof based on the user state or user states. Control may be local to a single module, shared across a cluster, or hierarchical.

In certain embodiments, the control system selects among multiple operating modes, including a capture mode, enrichment mode, regeneration mode, fractionation mode, polishing mode, purge mode, storage mode, blending mode, maintenance mode, and safe-shutdown mode. In certain embodiments, the control system selects among multiple feed streams or routes one feed stream to different concentration pathways. In certain embodiments, the control system may suspend or intensify a regeneration cycle based on current or forecasted renewable power, facility demand, or quality-control criteria.

In certain embodiments, the system includes alarms or interlocks that prevent or limit xenon administration or continued recapture operation when one or more safety thresholds are not met. In certain embodiments, a xenon-enabled room or bay may be actively purged or diluted if oxygen concentration falls below a selected threshold, if xenon concentration outside an intended area exceeds a selected threshold, if recapture flow is not verified, or if a module is saturated or improperly seated. In certain embodiments, patient-circuit isolation is preserved by one-way valves, compliant buffers, pressure regulators, or dedicated interfaces.

In certain embodiments, purge operation uses a clean-air pathway, an oxygen-containing pathway, or another purge source physically isolated from a xenon recapture path.

### Operational Methods for Xenon-Enabled Sites

In certain embodiments, a facility operator, site operator, or other system operator performs a method comprising establishing a controlled breathing zone in a xenon-enabled space, delivering a xenon-containing gas mixture to the controlled breathing zone without requiring a fully closed recirculating breathing loop, capturing at least a first portion of exhaled or leaked xenon via a local capture path positioned adjacent the controlled breathing zone, and routing air from the xenon-enabled space through a delayed local environmental recapture path during or after administration. The delayed local environmental recapture path may include one or more room recirculators, purifier loops, return-air paths, return-duct paths, exhaust paths, or combinations thereof.

In certain embodiments, a facility operator, site operator, clinician, or other administration-site actor performs a method that ends before regeneration. By way of example, the administration-site actor may establish the controlled breathing zone, deliver the xenon-containing gas mixture, capture at least a portion of exhaled or leaked xenon via a local capture path positioned adjacent the controlled breathing zone, and optionally route xenon-bearing air through a delayed local environmental recapture path, after which one or more loaded xenon recovery modules, inserts, vessels, or other xenon-bearing articles are removed, sealed, docked, packaged, or otherwise handed off for later regeneration, extraction, certification, blending, storage, reuse, or combinations thereof by another actor. The administration-site workflow therefore remains part of the present disclosure even when later regeneration, quality assurance, certification, or redistribution occurs at a different time, place, or by a different party.

In certain embodiments, the method further comprises regenerating at least one xenon recovery module that received xenon from the local capture path or the delayed local environmental recapture path, determining at least one quality attribute of gas released during regeneration, and routing at least a portion of the released gas to storage, blending, certification, repolishing, or reuse based on the quality attribute. In certain embodiments, regeneration occurs on-site. In certain embodiments, regeneration occurs off-site after a controlled transport or handoff of one or more loaded modules. In certain embodiments, the same site operator controls the delivery environment, the local capture path, the delayed recapture path, and the regeneration workflow. In other embodiments, one or more of those functions are performed by different actors while remaining part of the same physical xenon recovery ecosystem.

In certain embodiments, the operational method includes one or more safety, purge, and release-gating steps. By way of example, xenon delivery may be suspended, interrupted, or prevented unless a selected minimum oxygen condition is met; a purge mode may be initiated at session end or in response to a fault; and a recovered xenon-bearing fraction may be routed differently depending on its measured or inferred quality state, intended use class, or certification state. These operational methods are applicable to hospital rooms, operating rooms, recovery bays, clinics, dental operatories, wellness environments, transport environments, home environments, offices, and other xenon-enabled spaces.

In certain embodiments, analogous operational methods are used in biological-conditioning environments containing produce, seeds, tissues, livestock, companion animals, or other biological targets, with xenon delivery, recirculation, recapture, monitoring, purge, and later regeneration performed during storage, transport, cultivation, veterinary care, or other biological-handling workflows.

### Respiration-Synchronized Control and Wearable User-State Monitoring

In certain personal, wearable, or portable embodiments, xenon delivery, xenon capture, blower or pump duty cycle, valve timing, regeneration timing, or combinations thereof are synchronized to an actual, inferred, or predicted respiration-phase state. Respiration-phase state may be determined from one or more pressure signals, flow signals, acoustic signals, thermal signals, gas signals, chest-motion signals, accelerometry signals, photoplethysmography signals, impedance signals, or combinations thereof. In certain embodiments, xenon delivery is increased or initiated during inhalation or predicted inhalation and reduced or interrupted during exhalation or predicted exhalation. In certain embodiments, xenon capture is increased during exhalation or predicted exhalation. In certain embodiments, the controller modulates xenon delivery, xenon capture, or both based on one or more respiration-phase states, oxygen states, xenon states, user states, or combinations thereof.

In certain embodiments, the controller additionally monitors or infers one or more user-state variables including oxygen saturation, respiratory rate, delivered xenon concentration, exhaled xenon concentration, motion state, sleep state, relaxation state, exertion state, supervisory mode, or combinations thereof, and modifies delivery, capture, purge, alarms, authorization, or mode selection accordingly. In certain embodiments, the one or more user-state variables further include electroencephalography (EEG) signals, derived EEG features, other neurological or electrophysiological signals, heart-rate state, heart-rate-variability state, blood-pressure state, perfusion state, skin-conductance state, temperature state, arousal state, sedation state, level-of-consciousness state, clinician-entered target state, or combinations thereof. In certain embodiments, the controller modifies delivered xenon concentration, target xenon range, target xenon trajectory, session duration, alarms, authorization, or combinations thereof based on such user-state variables. In certain embodiments, the controller is configurable to operate in a first personal-use mode and a second supervised mode differing in one or more xenon concentration limits, monitoring requirements, authorization requirements, purge criteria, capture criteria, or combinations thereof.

### Power, Deployment, Distributed Systems, and Co-Processing

In certain embodiments, the xenon capture platform is powered by one or more of solar energy, wind energy, stored electrical energy, grid power, generator power, waste heat, recovered process energy, or combinations thereof. In certain embodiments, the platform is fully off-grid. In certain embodiments, it is hybrid. In certain embodiments, it is primarily grid-powered but uses renewables opportunistically.

In certain embodiments, the platform is implemented as a clustered, networked, or distributed arrangement of nodes. Such nodes may be physically co-located or geographically separated. In certain embodiments, each node performs first-stage capture and one or more downstream functions are centralized, such as regeneration, certification, blending, storage, or distribution. In certain embodiments, some nodes operate primarily in ambient-air mode, while others operate primarily in facility-recapture or locally enriched mode. In certain embodiments, the platform may aggregate recovered xenon-bearing fractions from multiple nodes into one inventory and one downstream quality-assurance architecture.

In certain embodiments, the xenon capture platform is co-processed with air cleaning, atmospheric water harvesting, carbon-dioxide handling, oxygen assurance, oxygen concentration, or combinations thereof. Such embodiments may derive benefit from shared air handling, shared pretreatment, shared heat management, shared logistics, or coproduct value. In certain embodiments, xenon capture is integrated with HVAC systems, air purifiers, mechanical-room air handlers, or environmental-control systems. In certain embodiments, xenon capture is integrated with atmospheric water harvesting or moisture-handling systems such that water removal and xenon capture share conditioning infrastructure. In certain embodiments, xenon capture is integrated with carbon-dioxide-handling infrastructure such that carbon-dioxide reduction occurs upstream of, alongside, or downstream of xenon-selective capture stages.

### REPRESENTATIVE EMBODIMENTS

### Wind-Powered Autonomous Harvester

In one non-limiting embodiment, the capture platform comprises a vertical-axis wind turbine having one or more blade-adjacent or blade-integrated air pathways, a central collection region, one or more xenon-selective or pretreatment stages, one or more regeneration subsystems, and one or more storage, blending, or output subsystems. Ambient air is routed through or adjacent to the rotating structure, conditioned, passed through one or more xenon-selective stages, and then routed toward a central or downstream concentration path. The turbine simultaneously supplies mechanical or electrical power to the system. In certain variants, oxygen-rich or conditioned side streams are retained for later blending or for other use. In certain variants, condensed water, dried air, or conditioned air are coproducts. The exact airflow geometry may vary widely and may include hollow blades, external ducts, central swirl chambers, auger-like internal flow paths, or related flow-shaping structures.

In certain variants, the rotor or turbine provides airflow handling, preconditioning, or power while xenon selectivity is provided primarily in a fixed or removable xenon-selective stage positioned in a blade-adjacent, nacelle, central-axis, or downstream flow path rather than by centrifugal separation alone.

### Modular Non-Turbine Xenon Harvester

In another non-limiting embodiment, the platform comprises a modular non-turbine xenon harvester including a high-flow intake or routing system, a pretreatment subsystem, a xenon-selective separation stage, a regeneration subsystem, an intermediate collection subsystem, and an optional compression and/or storage subsystem. One representative first prototype configuration of this branch comprises a substantially horizontal shell containing structured xenon-selective media and operated in adsorption and regeneration cycles with vacuum-assisted collection. Other forms may employ laminates, monoliths, radial-flow cartridges, purifier modules, duct modules, or other structured contactors rather than tray supports. This representative first prototype configuration is illustrative only and does not limit the broader modular non-turbine branch.

### Tray-in-Shell Worked Embodiment

In one non-limiting worked embodiment, a xenon-selective adsorption stage is housed in a substantially horizontal shell or fuselage carrying multiple tray-supported xenon-selective media layers. A gas stream is drawn through the shell during adsorption. After a selected adsorption interval, the internal volume of the shell is isolated, evacuated, and heated. Xenon-bearing gas released from the media is routed to an intermediate vessel, optionally with staged off-gassing or forecut handling, and is then routed to compression, storage, further concentration, or combinations thereof. In certain embodiments, the media comprise Ag-SSZ-13 carried on coated stainless mesh trays. In certain embodiments, heating is supplied by external drum heaters, shell heaters, or other thermal elements. In certain embodiments, a dry scroll pump, intermediate collection vessel, and booster compressor are used. This worked embodiment is expressly illustrative and is not intended to limit the broader concentration core to a tray shell, a specific shell volume, a specific heater arrangement, or a specific adsorbent chemistry.

### Structured Laminate Rapid-Swing Embodiment

In another representative embodiment, a xenon-selective medium is embedded in or coated onto a foil, laminate, monolith, or heat-exchanger-like support to create a low-pressure-drop structured contactor. The contactor is operated in rapid-cycle or intensified swing mode, optionally using direct electrical heating, embedded conductive traces, or other low-inertia regeneration techniques. A first structured module may function as a front-end collector, while a second smaller structured module functions as a secondary concentrator or microvolume collector. In certain embodiments, multiple such modules are operated in staggered adsorption and regeneration states.

### Scavenging Cartridge and Regeneration Station

In one non-limiting embodiment, a hospital, surgical center, or clinic installs replaceable xenon capture cartridges in one or more waste anesthetic gas scavenging pathways. Each cartridge contains a first region for carbon dioxide and/or moisture handling and a second region with xenon-selective media. Following use, the cartridge is removed and placed in a regeneration station located in a utility area. The regeneration station heats the cartridge, desorbs the xenon-bearing fraction, cools or reconditions the cartridge, and returns the cartridge to service or storage. The desorbed xenon-bearing gas is routed to an intermediate vessel, storage tank, cylinder, blending manifold, or concentration core for further handling.

### Indoor Purifier and Room Recirculation Network

In another non-limiting embodiment, one or more air purifiers or room recirculators in a clinic, office, dental practice, laboratory, or other enclosed environment are modified to include xenon-selective capture modules. Each purifier continues to perform conventional air-cleaning functions and also recovers xenon from the room air during or after xenon use. Saturated modules are exchanged and regenerated on-site or off-site. Multiple such devices may be distributed throughout a site and coordinated as a distributed recapture network.

### Transparent Recliner Canopy with Perimeter Capture

In one non-limiting embodiment, a seated or reclined user rests in a chair or lounger beneath a transparent or semi-transparent canopy. A xenon-containing mixture is introduced through an upper plenum or distributed diffuser to create a low-resistance controlled breathing zone near the user's face. A perimeter capture ring or edge slot positioned near the lower canopy opening removes leaked and exhaled xenon. The canopy may be open-bottom or partially open to permit rapid ingress and egress, while still supporting effective local capture. A post-session purge mode clears remaining xenon from the canopy volume and routes it toward a capture module or facility return path. In certain embodiments, the canopy geometry, perimeter capture geometry, purge timing, and recapture routing may be adapted for bedside, clinic, home, transport, or other semi-open deployments while preserving the same basic architecture of controlled breathing-zone delivery, local capture, delayed local environmental recapture, and purge operation.

### Air-Curtain Virtual-Mask Embodiment

In another non-limiting embodiment, one or more delivery nozzles or plenums produce a shaped xenon-containing flow that forms an aerodynamic breathing zone around the face without requiring a sealed mask. A rear plenum, side plenum, or perimeter extraction path captures the resulting local flow field and exhaled xenon. The system may transition between xenon-delivery mode and clean-air purge mode depending on session state or safety state. This embodiment may be incorporated into a chair, headrest, bedside support, clinic workstation, transport seat, or other installation.

### Dental Face-Zone Dual-Capture Embodiment

In another non-limiting embodiment, a patient in a dental or clinic chair receives xenon through a low-profile patient-zone delivery structure while a first capture path removes xenon near the face and a second capture path recovers residual xenon from the room or operatory recirculation environment. The first capture path may include an extraoral suction arm, circumoral return, open shield with integrated extraction, or similar local capture hardware. The second capture path may include a purifier-integrated capture module, room recirculator, return-air module, or other delayed local environmental recapture subsystem.

In certain embodiments, any xenon delivery interface, whether sealed, substantially sealed, semi-open, open-headspace, canopy-based, cannula-based, mask-based, circuit-based, or otherwise configured to deliver a xenon-containing gas mixture to a user, is paired with one or more facility capture paths positioned in or fluidly coupled to a surrounding environment. The one or more facility capture paths may include one or more scavenging lines, room recirculators, air purifiers, return grilles, return ducts, exhaust paths, mechanical-room paths, or combinations thereof, and may recover xenon that escapes the xenon delivery interface for later regeneration, concentration, certification, blending, storage, reuse, or combinations thereof.

### Off-Site Regeneration Service Loop

In another non-limiting embodiment, a facility does not regenerate capture modules on-site. Instead, loaded modules are sealed or otherwise prepared for transport and shipped or otherwise transferred to a regeneration center. The regeneration center receives the modules, verifies identity and condition, regenerates the modules, performs quality assurance on the recovered xenon-bearing fraction, and either returns regenerated modules and certified xenon product separately or routes the certified xenon product into a separate distribution channel. The capture facility therefore participates in the ecosystem even though regeneration and certification occur elsewhere. In certain embodiments, the off-site regeneration workflow includes one or more chain-of-custody states, separate module-return and product-return workflows, and compatibility-controlled release of regenerated xenon-bearing product based on one or more quality attributes.

In certain embodiments, a regeneration center, service provider, utility operator, or other downstream actor performs a method comprising receiving one or more loaded xenon recovery modules from a xenon-enabled site, regenerating or otherwise processing the one or more loaded xenon recovery modules to release a xenon-bearing fraction, determining or accessing at least one quality attribute of the xenon-bearing fraction, and routing the xenon-bearing fraction to storage, blending, certification, reuse, repolishing, discard, or combinations thereof based on the at least one quality attribute. In certain embodiments, the regeneration-site method is operationally distinct from the administration-site method, and the different methods are performed by different actors within a shared xenon recovery ecosystem.

### Permanent Installed HVAC or Mechanical-Room Module

In another non-limiting embodiment, a permanently installed xenon recovery module is positioned in a return duct, return grille, mechanical room, or other stationary facility capture path. The module includes one or more xenon-selective stages and is regenerated in situ or through a service process. Downstream quality assurance, certification, and release gating are the same or substantially the same as for a removable cartridge embodiment.

### Keyed Concentration-Class Release Architecture

In another non-limiting embodiment, a recovered xenon-bearing product or blended xenon-containing mixture is stored in a pressure vessel, bottle, cartridge, or cylinder having a keyed or indexed interface corresponding to a selected concentration class or intended use class. A compatible filling connector, docking interface, or dispensing interface confirms compatibility before filling or dispensing. A certification gate may additionally require that the product meet a selected oxygen threshold, xenon concentration threshold, or quality class before the keyed interface is unlocked, energized, or otherwise released.

### Produce Preservation and Transport Container

In another non-limiting embodiment, harvested produce or other plant material is stored or transported within a storage room, pallet enclosure, refrigerated trailer, shipboard container, or other controlled volume. A xenon delivery subsystem establishes or replenishes a xenon-containing atmosphere within the controlled volume or a recirculating branch thereof at ambient or near-ambient pressure. Gas withdrawn from the controlled volume is routed through one or more xenon recovery inserts or xenon recovery modules during or after a conditioning interval and recovered xenon is routed to regeneration, storage, blending, or reuse. In certain variants, controller logic coordinates xenon with temperature, humidity, carbon dioxide, oxygen, ethylene, ripening state, spoilage state, or combinations thereof.

### Veterinary Holding or Livestock Transport Enclosure

In another non-limiting embodiment, a veterinary holding environment, kennel, stall, livestock compartment, animal transport bay, or recovery enclosure is configured to deliver xenon through a semi-open local breathing zone, enclosure headspace, or conditioned recirculation flow while oxygen assurance, purge capability, local capture, delayed local environmental recapture, or combinations thereof are maintained. The embodiment may be used for calm transport, peri-procedural support, recovery, stress mitigation, or other animal-care contexts.

### Comfort-Centered Care Environment

In another non-limiting embodiment, a semi-open xenon administration environment is adapted for palliative, hospice, veterinary, or other legally authorized end-of-life or terminal-care contexts in which reduced distress, quiet breathing, caregiver access, and a calm surrounding environment are important. The environment may use the same controlled breathing zone, oxygen assurance, purge, local capture, delayed local environmental recapture, monitoring, and backend recovery architectures described elsewhere herein while clarifying that the platform is not limited to curative, performance, or recovery-oriented use.

### Distributed Wearable Personal Xenon System

In one non-limiting embodiment, a personal xenon system is distributed across multiple body-worn or user-carried subsystems. A lightweight face-adjacent delivery interface establishes a localized xenon-enriched breathing microenvironment adjacent the nose and/or mouth without requiring a tight face seal, while a separate local capture subsystem positioned at a collar, neck, chest, shoulder, or garment region intercepts exhaled or leaked xenon before full ambient dilution. One or more flexible conduits couple the delivery and capture subsystems to a user-carried subsystem containing a source vessel, xenon recovery module, gas mover, valves, controller, battery, dehumidification subsystem, regeneration subsystem, or combinations thereof. By separating face-adjacent elements from heavier process elements, the embodiment may improve weight distribution, comfort, thermal management, mobility, discretion, and user adherence while preserving the same core delivery, recapture, regeneration, monitoring, and reuse architecture described elsewhere herein. The exact distribution of mass and function may vary widely and may place heavier elements in a pendant, chest pod, vest, jacket, harness, belt pack, backpack, bedside support, or other user-carried structure.

### Gaiter/Collar/Lower-Face Hybrid Personal Interface

In another non-limiting embodiment, a personal xenon interface is formed as a hybrid lower-face and neck architecture comprising one or more of a nasal interface, nostril interface, circumoral frame, transparent lower-face shell, collar-like boundary, gaiter-integrated structure, scarf-or wrap-integrated structure, textile-integrated structure, or combinations thereof. A delivery element establishes a xenon-enriched breathing microenvironment adjacent to the user's nose and/or mouth, while one or more lower-face, neck, or collar capture inlets receive exhaled or leaked xenon for routing to a xenon recovery module. In certain variants, the same hybrid structure includes integrated sensing, flow shaping, humidification, draft management, acoustic management, visual-transparency features, or combinations thereof. In certain variants, the structure preserves visibility of at least a portion of the user's face and permits speech, ambulation, work, rest, or social interaction during xenon delivery, thereby providing a personal interface that is less stigmatizing, less claustrophobic, and more compatible with non-clinical use settings than conventional closed masks or tightly sealed breathing hardware. The exact ergonomic form may vary widely and may be rigid, semi-rigid, textile-based, flexible, articulated, transparent, translucent, or hybrid.

### Chest-Pod or Pendant Exhalation Receiver

In another non-limiting embodiment, a local exhalation receiver is positioned at a chest, sternum, clavicle, pendant, collar, or other body-adjacent location such that natural exhalation, downward-directed exhalation, or user-directed exhalation is received before full ambient dilution. The receiver may comprise a chest pod, pendant housing, collar receiver, slot inlet, flared inlet, funnel-like inlet, ring structure, plume receiver, or combinations thereof, and may be passive, actively assisted by suction, or selectively actuated during exhalation or predicted exhalation. Gas received by the exhalation receiver is routed through one or more conduits to dehumidification, xenon-selective recovery, temporary storage, regeneration, monitoring, or combinations thereof. In certain variants, the user may exhale normally for convenience, exhale more deliberately toward the receiver when higher recovery efficiency is desired, or alternate between such modes. In certain variants, the receiver is dockable with a cradle, charging station, regeneration station, refill station, or data interface for later charging, service, xenon recovery, synchronization, or combinations thereof.

### Personal Humidity-Recycling Xenon System

In another non-limiting embodiment, a personal xenon system includes a moisture-management subsystem positioned upstream of a xenon-selective recovery subsystem such that water vapor in captured exhaled gas is reduced before contact with one or more xenon-selective media. Moisture reduction may occur by condensation, desiccant capture, membrane separation, sorption, a dehumidification wheel, or combinations thereof. Xenon recovered from the resulting drier gas is retained in one or more xenon-selective media and later released by heating, pressure reduction, mild vacuum, or combinations thereof into a reduced-volume collection region configured to limit dead-volume dilution. In certain variants, water removed from the exhaled gas is stored, condensed, sorbed, or otherwise recovered and later routed to a humidification subsystem that humidifies gas delivered back to the user, thereby improving comfort, reducing dryness, and allowing user-derived moisture to participate in a partial comfort loop. In certain variants, the same thermal or control architecture is used to coordinate moisture recovery, xenon release, reconcentration, blending, storage, and later delivery.

### Supervised Personal-to-Clinical Continuum Embodiment

In another non-limiting embodiment, a personal or body-worn xenon platform is operable in a continuum of modes ranging from personal-use enrichment or wellness operation to more tightly controlled supervised operation using substantially the same core architecture. A first mode may be configured for home use, sleep or relaxation support, recovery, travel, or other personal contexts with selected concentration limits, portable recapture, user-state monitoring, and simplified workflows. A second mode may be configured for supervised clinical, peri-procedural, post-procedural, or higher-acuity use with tighter concentration control, enhanced monitoring, additional authorization or lockout logic, clinician override, stricter purge criteria, richer sensing, more formal data logging, or combinations thereof. In certain variants, the same delivery interface family, local capture subsystem, dehumidification subsystem, xenon recovery module, regeneration subsystem, and docking ecosystem are used across both modes, while software state, hardware population, connected accessories, or compatibility-controlled interfaces differentiate authorized use classes. This embodiment supports a scalable product family in which a common xenon architecture spans consumer, professional, and supervised settings without requiring conventional anesthesia-machine architecture in every mode.

### Variants, Equivalents, and Future-Proofing

The xenon platform disclosed herein is not limited to one feed regime, one biological target class, one machine format, one sorbent class, one membrane class, one power source, one facility type, one regeneration location, or one business model. Various embodiments may employ different xenon-selective media, contactor geometries, regeneration modalities, interface types, recapture locations, deployment architectures, and workflow arrangements, including the representative examples described herein.

True ambient-air capture remains part of the present disclosure as a foundational, future-improvable embodiment. Conditioned ambient air, recirculated or locally enriched atmospheres, and higher-concentration waste or process streams are equally within the scope of the present disclosure and may become preferred operating regimes under current materials and process economics. As xenon-selective adsorbent materials, membrane materials, dehydration technologies, structured contactor fabrication methods, and regeneration techniques continue to advance, whether through incremental optimization, new material classes, computational materials discovery, or fundamental breakthroughs, the systems and methods disclosed herein are expected to benefit directly and proportionally from such advances. The disclosed architecture is designed to be the beneficiary of, rather than limited by, future improvements in any one precursor technology or component capability, because the system-level integration, process workflows, multi-regime adaptation, fraction-cut enrichment, recapture strategies, and administration environments are defined by architectural relationships that are independent of the specific performance level of any one underlying material or subsystem. Accordingly, the present disclosure is intended to encompass embodiments in which one or more precursor technologies—including but not limited to adsorbent capacity, adsorbent selectivity, membrane permeance, dehydration efficiency, regeneration speed, thermal management capability, sensor sensitivity, or controller sophistication—are materially superior to those available at the time of filing, and in which such improvements translate into higher throughput, lower energy consumption, smaller footprint, lower cost, or expanded application domains for the disclosed systems and methods.

### Atmospheric Water Harvesting Co-Processing

In certain embodiments, the water removed during the dehydration of feed gas for xenon capture is treated as a co-product rather than a waste stream. At a processing rate of approximately 1,000 cubic feet per minute under conditions of approximately 25 degrees Celsius and 50 percent relative humidity, the dehydration subsystem may remove approximately 15 to 25 kilograms per hour of water from the air stream. In arid regions where potable or agricultural water is scarce, this water may be collected, filtered, and stored for beneficial reuse, offsetting part or all of the operating cost of the dehydration subsystem. In certain embodiments, the condensed water is routed to a collection vessel, optionally passed through a particulate or biological filter, and stored for local use, agricultural irrigation, cooling-system makeup, or other purposes. In certain embodiments, the water co-product is used within the xenon platform itself, for example as cooling water, as a thermal ballast, as humidification feedwater for delivered gas in an administration environment, or as a source of distilled water for clinical use. The integration of atmospheric water harvesting with xenon capture represents a synergistic co-processing opportunity in which the energy expended for dehydration produces two valuable products—dry feed gas for xenon capture and recovered water —rather than one. In certain embodiments, the economic value of the recovered water in arid environments materially improves the overall economic return of the xenon capture installation.

### Unified Platform Continuum: Industrial Bulk Harvesting Through Clinical Delivery

The systems and methods disclosed herein are expressly designed to span a continuous operational continuum from large-scale industrial bulk xenon harvesting in geographically optimized outdoor locations through medium-scale facility-integrated capture and recapture, to small-scale clinic, wellness, dental, veterinary, home, or personal-use environments optimized for the administration of xenon to biological targets. This continuum is not merely an enumeration of separate inventions but reflects a unified platform architecture in which the same core principles—multi-regime xenon-selective capture, staged concentration with fraction-cut enrichment, thermal integration of dehydration with regeneration, buoyancy-assisted containment, and recapture from semi-open administration environments—apply at every scale, with the specific parameters (bed mass, contactor area, blower capacity, dehydration staging, cycle timing, product handling, and administration geometry) scaled according to throughput requirements and feed conditions.

At the industrial-harvesting end of the continuum, installations comprising arrays of approximately 10 to 100 or more contactor modules, each of approximately 0.25 to 1.0 square meters of cross-sectional area, process ambient air at rates of approximately 10,000 to 100,000 cubic feet per minute or more at geographically favorable sites, producing approximately 50 to 200 liters or more of xenon per day for bulk storage, distribution, or on-site conversion to certified products. At the facility-integration end, capture modules integrated into HVAC systems, return-air ducts, or mechanical-room skids process recirculated or semi-enclosed atmospheres at moderate flow rates, recovering xenon that would otherwise be lost to exhaust or dilution. At the clinical-delivery end, semi-open administration pods, air-curtain delivery systems, dental operatory systems, wearable devices, and personal breathing microenvironments deliver xenon to individual users at concentrations of approximately 10 to 70 percent with local recapture efficiencies of approximately 85 to 99 percent. The xenon captured at any point on this continuum may feed any other point: bulk-harvested xenon supplies clinical delivery; clinical recapture feeds regeneration and reuse; facility-integrated capture supplements purchased supply; and the logistics, certification, quality-assurance, and chain-of-custody infrastructure disclosed herein coordinates the flow of xenon across the continuum.

This unified continuum approach is non-obvious because conventional xenon infrastructure treats bulk production (cryogenic air separation) and point-of-use delivery (anesthesia machines with sealed circuits) as entirely separate industries with separate technology stacks, separate supply chains, and no architectural integration. The present disclosure reconceives these as a single platform family in which the same underlying principles of selective adsorption, thermal-swing regeneration, fraction-cut enrichment, and recapture economics apply across all scales and feed regimes.

### Illustrative Embodiments and Predicted Performance

The following illustrative embodiments describe predicted performance characteristics based on first-principles thermodynamic analysis and published adsorption data for the classes of materials disclosed herein. These embodiments are provided to further illustrate the principles of the invention and are not intended to limit the scope of the claims.

### Illustrative Embodiment A: Ambient-Air Xenon Capture Module

In one illustrative embodiment, a xenon recovery module is configured for true ambient-air capture and comprises a blower rated at approximately 1000 cubic feet per minute, a pretreatment subsystem including a particulate filter and a regenerable desiccant stage configured to reduce inlet moisture to below approximately 100 parts per million, and a xenon-selective adsorption stage comprising three beds of approximately 10 kilograms each of silver-exchanged zeolite material (such as Ag-ETS-10 or Ag-SSZ-13) disposed in a structured monolith or laminate contactor geometry. The three beds are operated in a staggered thermal-swing adsorption cycle such that at least one bed is in adsorption mode, at least one is in regeneration mode, and at least one is in cooling or standby mode at any given time. Regeneration is performed at a temperature in the range of approximately 200 to 220 degrees Celsius under moderate vacuum, with a cycle period of approximately 10 to 30 minutes per bed. Based on published adsorption data for silver-exchanged zeolites at atmospheric xenon levels, each adsorption-regeneration cycle is predicted to yield approximately 15 to 30 milliliters of xenon at standard temperature and pressure per 10 kilogram bed, corresponding to a daily production rate of approximately 1.5 to 3.5 liters of xenon at standard conditions, approaching the feed-limited ceiling of approximately 3.25 liters per day at 1000 cubic feet per minute and 87 parts per billion atmospheric xenon.

### Illustrative Embodiment B: Semi-Open Administration Pod with Local Recapture

In another illustrative embodiment, a semi-open administration apparatus defines a controlled breathing zone of approximately 35 to 50 liters of internal free-gas volume around a user's head and upper torso. The apparatus comprises an open-front clamshell enclosure, a gas-curtain delivery system configured to deliver a xenon-oxygen mixture from slots positioned at approximately the brow and temple regions at a total flow rate of approximately 15 to 25 cubic feet per minute and a slot velocity of approximately 0.5 to 1.5 meters per second, upper return ports positioned to capture the initial expiratory jet, lower return ports positioned to capture dense xenon-enriched gas that tends to settle by gravity, and a recirculation loop comprising a carbon dioxide scrubber, an oxygen top-up stage, and a water management stage. In this embodiment, the primary recirculation loop maintains a xenon-oxygen mixture at a target xenon concentration and continuously removes metabolic carbon dioxide and excess moisture without routing the bulk loop gas through a zeolite adsorption stage. A secondary leak-capture stream of approximately 3 to 8 cubic feet per minute is drawn from the periphery of the breathing zone and routed to a xenon recovery module for recapture. Between sessions, the loop gas inventory of approximately 40 to 60 liters is recovered into a pressurized buffer vessel at approximately 4 to 6 bar for reuse in subsequent sessions, with a target recovery of approximately 99 percent or greater. Based on published data for closed-circuit xenon delivery systems, this architecture is predicted to achieve maintenance xenon consumption rates comparable to or lower than approximately 2 to 4 liters per hour at xenon concentrations of approximately 30 to 70 percent in the breathing zone.

### Illustrative Embodiment C: Geographically Optimized Wind-Driven Ambient Harvester

In another illustrative embodiment, a xenon recovery system is configured for deployment at a geographically favorable site exhibiting sustained high wind speeds and sub-ambient temperatures, such as a katabatic wind corridor in a polar or subpolar coastal region where mean wind velocities are approximately 20 to 22 meters per second and mean ambient temperatures are approximately 250 to 260 Kelvin (approximately minus 23 to minus 13 degrees Celsius). The system comprises a stagnation plenum or wind-catcher inlet aligned with the prevailing wind direction and configured to recover wind dynamic pressure of approximately 150 to 340 Pascals, a structured monolith contactor having a cross-sectional area of approximately 0.25 square meters, a cell density of approximately 300 to 500 cells per square inch, and a pressure drop of no greater than approximately 200 Pascals at a superficial velocity of approximately 2 meters per second, such that the wind dynamic pressure is sufficient to drive air through the contactor without any active fan or blower power. The structured contactor is loaded with silver-exchanged zeolite material (such as Ag-ETS-10, Ag-SSZ-13, or Ag-ZSM-5) whose effective working capacity is enhanced by a factor of approximately 15 to 30 times relative to operation at 298 Kelvin due to the sub-ambient temperature, in accordance with the van't Hoff equation for exothermic adsorption processes with an isosteric heat of adsorption of approximately 40 to 50 kilojoules per mole.

At the sub-ambient temperatures characteristic of this deployment, the ambient dew point is well below approximately minus 73 degrees Celsius, and the ambient air is naturally drier than the moisture threshold required for effective silver-exchanged zeolite operation, such that no active dehydration stage is required. The elimination of the dehydration subsystem removes the dominant energy load of temperate-climate ambient capture, which is estimated at approximately 9 to 10 kilowatt-hours per liter of xenon at standard temperature and pressure in humid temperate conditions, and reduces total system energy to the thermal regeneration component and parasitic control loads.

In this embodiment, the passive wind-driven volumetric flow rate through the 0.25 square meter contactor at a superficial velocity of approximately 2 meters per second is approximately 0.5 cubic meters per second, or approximately 30 cubic meters per minute. At the atmospheric xenon mole fraction of approximately 87 parts per billion and a xenon capture efficiency of approximately 80 to 95 percent, the xenon capture rate is approximately 0.10 to 0.13 millimoles per minute. Over a continuous harvesting cycle of approximately 7 days (approximately 10,080 minutes), the total xenon captured per contactor module is approximately 1.0 to 1.3 moles, corresponding to approximately 22 to 29 liters at standard temperature and pressure. Regeneration is performed at the end of each cycle by heating the contactor mass of approximately 30 to 40 kilograms (sorbent plus housing) from the ambient temperature of approximately 253 Kelvin to approximately 490 Kelvin, requiring a sensible heat input of approximately 2.0 to 2.5 kilowatt-hours per cycle, plus a parasitic control load of approximately 50 Watts continuous (approximately 8.4 kilowatt-hours per week). The total energy consumption per cycle is therefore approximately 10 to 11 kilowatt-hours, yielding a specific energy of approximately 0.3 to 0.5 kilowatt-hours per liter of xenon at standard temperature and pressure.

This specific energy is approximately 5 to 90 times lower than the marginal incremental energy for xenon recovery from an existing cryogenic air separation unit (approximately 2 to 27 kilowatt-hours per liter), and approximately 1,000 to 7,000 times lower than the full-allocation energy for dedicated cryogenic xenon production from air (approximately 500 to 2,500 kilowatt-hours per liter). The energy advantage is robust: even if prevailing wind speed drops by approximately 50 percent, reducing the dynamic pressure quadratically and the volumetric flow proportionally, the weekly xenon yield decreases to approximately 8 to 10 liters per module while the fixed parasitic load causes the specific energy to rise to approximately 1.0 to 1.1 kilowatt-hours per liter—still substantially below the cryogenic baseline. In certain embodiments, multiple contactor modules of approximately 0.25 to 1.0 square meters each are arranged in parallel arrays of approximately 10 to 100 modules, sharing common regeneration infrastructure and product collection systems, to achieve aggregate production rates of approximately 50 to 200 liters of xenon per day at a single geographic installation.

### System Integration Synergy

The integrated architecture disclosed herein is configured to achieve performance exceeding the arithmetic sum of independently optimized subsystems. Based on first-principles thermodynamic, adsorption kinetic, fluid dynamic, and mass-balance analyses, the combination of (i) multi-regime feed adaptation, (ii) fraction-cut enrichment during thermal swing regeneration, (iii) cascaded thermal integration between dehydration subsystems and xenon sorbent regeneration, (iv) buoyancy-assisted semi-open containment exploiting the high molecular weight of xenon, and (v) rich-loop recirculation with room-level environmental recapture produces an overall system efficiency improvement of approximately 3 to 4 times compared to a system constructed from the same components without integration synergies. In certain embodiments, the operating cost per administration session, inclusive of xenon makeup, electrical energy, and consumables, is in the range of approximately 40 to 80 dollars per session at prevailing xenon prices, compared to an estimated 200 to 300 dollars per session for a non-integrated system of comparable capability. These performance characteristics are emergent properties of the integrated architecture and are not predictable from analysis of any subsystem in isolation.

In certain embodiments, the synergy factor remains above approximately 3 across a range of operating conditions, including variations in xenon purchase price, electricity cost, session frequency, room recovery efficiency, and environmental humidity, indicating that the integration advantage is a robust architectural property rather than an artifact of favorable assumptions at a single operating point. When the capture subsystem is deployed at geographically optimized sites as described in Illustrative Embodiment C, the integration synergy extends further: the geographic elimination of dehydration energy, combined with wind-driven airflow and temperature-enhanced sorbent capacity, compounds multiplicatively with the downstream integration advantages of fraction-cut enrichment, buoyancy-assisted containment, and rich-loop recirculation, such that the total system energy budget from atmospheric capture through patient administration may be reduced by an additional factor of approximately 10 to 100 relative to a geographically non-optimized but architecturally integrated system.

What is claimed is:

1. A system for recovering and reusing xenon, the system comprising:
a xenon concentration subsystem configured to recover xenon from a xenon-containing gas mixture via at least one xenon-selective separation stage and to produce a xenon-enriched gas;
a delivery subsystem configured to deliver a xenon-containing gas to a controlled breathing zone adjacent to a user without requiring a fully closed recirculating breathing loop or a tight face seal on the user;
a recapture subsystem configured to recover xenon from at least one of (i) a local capture path adjacent to the controlled breathing zone or (ii) a delayed local environmental recapture path associated with a surrounding space;
a regeneration or xenon-release processing subsystem configured to release xenon captured by the recapture subsystem for storage, further concentration, or redelivery; and
a controller configured to coordinate operation of the xenon concentration subsystem, the delivery subsystem, the recapture subsystem, and the regeneration or xenon-release processing subsystem.

2. The system of claim 1, further comprising a pretreatment stage configured to reduce moisture, carbon dioxide, or both from the xenon-containing gas mixture upstream of the at least one xenon-selective separation stage, and wherein the xenon concentration subsystem comprises a staged concentration architecture having a primary xenon collector and a secondary concentrator with a collection region of smaller internal free-gas volume than the primary xenon collector.

3. The system of claim 1, wherein the controller is configured to operate the system in at least two of: (i) an ambient-air capture mode in which the xenon-containing gas mixture comprises ambient atmospheric air, (ii) an enriched-feed capture mode in which the xenon-containing gas mixture comprises a locally enriched or recirculated atmosphere, (iii) a scavenging or exhaust-capture mode in which the xenon-containing gas mixture comprises a scavenging stream or exhaust stream, and (iv) a session-end recovery mode, and wherein the controller is further configured to transition between modes based on at least one of measured xenon concentration, inferred feed composition, or session state.

4. The system of claim 1, wherein the at least one xenon-selective separation stage comprises at least one of: a membrane stage having a xenon-to-oxygen selectivity of at least 5 and a xenon permeance of at least 50 gas permeation units, a cryogenic micro-trap maintained at a temperature between 120 K and 150 K with an internal volume of less than 100 mL, or a hybrid combination thereof.

5. The system of claim 1, wherein the system is powered at least in part by wind power, solar power, or recovered process energy, and wherein at least a portion of gas movement through the xenon concentration subsystem is driven by wind-powered airflow without active fan or blower power during periods of sufficient wind velocity.

6. A xenon administration and recovery system comprising:
a delivery subsystem configured to create a controlled breathing zone adjacent to a user's nose, mouth, or both without requiring a fully closed recirculating breathing loop or a tight face seal on the user, the delivery subsystem being configured to deliver a xenon-containing gas to the controlled breathing zone at substantially ambient pressure;
a local capture subsystem positioned adjacent to the controlled breathing zone and configured to capture at least a portion of exhaled or leaked xenon-containing gas during administration;
a delayed local environmental recapture subsystem configured to recover xenon from a surrounding space associated with the administration during or after a delivery session;
a safety subsystem configured to verify that oxygen concentration in or adjacent to the controlled breathing zone remains above a selected threshold and to initiate a purge operation in response to a fault condition; and
a controller configured to coordinate operation of the delivery subsystem, the local capture subsystem, the delayed local environmental recapture subsystem, and the safety subsystem.

7. The system of claim 6, wherein the delivery subsystem comprises at least one of a delivery plenum, nozzle, diffuser, or manifold configured to generate a shaped flow field that forms or sustains the controlled breathing zone, and wherein the local capture subsystem comprises at least one of an extraction plenum, slot, ring, or return path positioned to capture at least part of the shaped flow field after passage through the controlled breathing zone.

8. The system of claim 6, wherein a xenon-containing gas mixture delivered to the controlled breathing zone has a density at least 1.5 times that of surrounding ambient air, and wherein the controlled breathing zone is configured such that buoyancy-assisted stratification retards upward dispersion of the xenon-containing gas mixture from the controlled breathing zone, the system achieving a Richardson number exceeding 10 in the controlled breathing zone during steady-state delivery.

9. The system of claim 6, further comprising a ventilation-interlock mode in which the controller communicates with building heating, ventilation, and air-conditioning controls to reduce or redirect supply-air flow within or adjacent to the controlled breathing zone during xenon delivery, and wherein a local-capture flow rate maintained by the local capture subsystem exceeds a cross-draft velocity within the controlled breathing zone by at least a factor of two.

10. The system of claim 6, wherein the delivery subsystem is configured to deliver a xenon-containing gas mixture to a patient while leaving an oral treatment field accessible to a clinician, and wherein the local capture subsystem comprises a first capture path adjacent to the patient and the delayed local environmental recapture subsystem comprises a second capture path in an operatory, treatment room, or surrounding space.

11. The system of claim 6, wherein xenon delivery concentration, xenon delivery flow rate, or both are modulated based at least in part on one or more user states selected from the group consisting of electroencephalography signals, oxygen-saturation state, respiratory rate, heart-rate-variability state, sedation state, relaxation state, and combinations thereof.

12. A regeneration and quality-management system for xenon recovery modules, the system comprising:
a docking interface configured to receive a xenon recovery module containing a xenon-selective capture medium bearing adsorbed or captured xenon;
a regeneration subsystem configured to release a xenon-bearing fraction from the xenon recovery module;
an analyzer or data subsystem configured to determine at least one quality attribute of the xenon-bearing fraction released from the xenon recovery module;
a release-control subsystem configured to classify the xenon-bearing fraction based on the at least one quality attribute and to authorize or restrict routing of the xenon-bearing fraction to a selected destination based on the classification; and
a compatibility-controlled interface configured to permit or restrict coupling, filling, transfer, or dispensing of a xenon-bearing gas based on the classification.

13. The system of claim 12, wherein the release-control subsystem is configured to route the xenon-bearing fraction to at least one destination selected from the group consisting of a local reuse pathway, a higher-purity pathway, a repolishing pathway, a blending manifold, and a discard pathway, and wherein the compatibility-controlled interface differentiates the at least one destination by physical geometry, coded connector, or electronic authorization.

14. The system of claim 12, further comprising a xenon-bearing gas vessel having a compatibility-controlled geometry cooperating with at least one external indicium corresponding to a concentration class, intended-use class, or certification state, wherein the compatibility-controlled geometry prevents coupling between vessels or interfaces of incompatible classes.

15. The system of claim 12, further comprising a transport or handoff pathway configured to move the xenon recovery module or a xenon-bearing gas between a first site at which xenon was captured and a second site at which the docking interface is located, and a chain-of-custody subsystem configured to track a state of the xenon recovery module through at least loaded, in-regeneration, certified, and returned-to-service states.

16. The system of claim 12, further comprising a service workflow in which the system receives a loaded xenon recovery module from a remote xenon-enabled site, regenerates the xenon recovery module, classifies the released xenon-bearing fraction, routes the classified fraction to a destination, and updates a chain-of-custody state associated with the xenon recovery module.

17. A personal xenon administration and recovery system comprising:
a personal delivery interface configured to create a localized xenon-enriched breathing microenvironment adjacent to a user's nose, mouth, or both without requiring a fully closed recirculating breathing loop or a tight face seal on the user;
a local capture subsystem positioned at or adjacent to a personal microenvironment region of the user and configured to intercept at least a portion of exhaled or leaked xenon-containing gas before full ambient dilution;
at least one xenon recovery module comprising a xenon-selective capture medium configured to recover xenon from gas intercepted by the local capture subsystem;
a dehumidification subsystem positioned upstream of the xenon-selective capture medium and configured to reduce moisture content of gas intercepted by the local capture subsystem; and
a controller configured to modulate xenon delivery, capture, or both based on at least one of respiration-phase state, oxygen state, xenon state, or user state.

18. The system of claim 17, wherein the system employs a distributed body-worn architecture in which a first subsystem is positioned adjacent the user's face and a second subsystem containing at least one of a source vessel, the at least one xenon recovery module, a pump, a battery, or the controller is positioned at a collar, pendant, chest pod, vest, belt pack, or backpack remote from the user's face.

19. The system of claim 17, wherein the local capture subsystem comprises an exhalation receiver configured to receive natural or user-directed exhalation, the exhalation receiver being positioned at a collar receiver, chest pod, pendant, shoulder-mounted inlet, or textile-integrated capture channel.

20. The system of claim 17, further comprising a moisture recycling loop wherein water removed by the dehumidification subsystem is recovered, stored, or routed to a humidification subsystem configured to humidify gas delivered to the user by the personal delivery interface.

21. The system of claim 17, further comprising a dockable regeneration subsystem configured to receive the at least one xenon recovery module or a portion thereof and to release recovered xenon into a collection region having a smaller internal free-gas volume than a housing volume of the at least one xenon recovery module, the dockable regeneration subsystem comprising a dock, cradle, or station configured for at least one of charging, regeneration, refill, or data exchange.

22. A xenon capture system comprising:
a wind-driven airflow structure configured to direct ambient air through an airflow path without requiring continuous active fan or blower power during periods of sufficient wind velocity;
a xenon-selective separation stage disposed in the airflow path and supported by a structured contactor configured to operate at a pressure drop of less than 500 pascals at a design flow rate;
a regeneration subsystem configured to release a xenon-bearing fraction from the xenon-selective separation stage; and
a collection or storage subsystem configured to receive and store the xenon-bearing fraction.

23. The system of claim 22, wherein the system is configured for deployment at a geographically selected site exhibiting at least two of: (i) a mean ambient temperature below a selected temperature threshold, (ii) a mean ambient relative humidity below a selected humidity threshold, and (iii) a mean wind speed above a selected wind-speed threshold, such that dehydration energy required upstream of the xenon-selective separation stage is reduced relative to a temperate-climate baseline.

24. The system of claim 22, further comprising a plurality of modular xenon capture units arranged in a parallel array, each modular xenon capture unit comprising an independent xenon-selective separation stage, and a shared regeneration, collection, or storage subsystem servicing the plurality of modular xenon capture units, wherein each modular xenon capture unit is independently serviceable.

25. The system of claim 22, further comprising at least one co-processing subsystem selected from the group consisting of an atmospheric water harvesting subsystem, a carbon-dioxide capture or management subsystem, an air purification subsystem, and an oxygen concentration subsystem, the at least one co-processing subsystem being integrated with or co-located with the xenon capture system.

26. A method of operating a xenon-enabled site, the method comprising:
delivering a xenon-containing gas mixture to a controlled breathing zone in a xenon-enabled space without requiring a fully closed recirculating breathing loop;
recovering xenon from at least one of (i) a local capture path adjacent to the controlled breathing zone or (ii) a delayed local environmental recapture path associated with the xenon-enabled space;
regenerating or otherwise processing at least one xenon recovery module that captured the recovered xenon to release a xenon-bearing fraction;
determining at least one quality attribute of the xenon-bearing fraction; and
routing the xenon-bearing fraction to at least one of storage, reuse, blending, further concentration, or certification based on the at least one quality attribute.

27. The method of claim 26, wherein regenerating comprises separating the xenon-bearing fraction into at least a first fraction and a second fraction based on at least one of time, temperature, pressure, or detected composition, and collecting the second fraction in a collection region having a smaller internal free-gas volume than a housing of the at least one xenon recovery module.

28. The method of claim 26, further comprising verifying that oxygen concentration in or adjacent to the controlled breathing zone remains above a selected threshold during delivering, and initiating a purge operation in response to at least one of a fault condition, an occupancy change, a capture-path fault, or a ventilation fault.

29. The method of claim 26, further comprising receiving one or more loaded xenon recovery modules from a remote xenon-enabled site, regenerating the one or more loaded xenon recovery modules, classifying the released xenon-bearing fraction based on the at least one quality attribute, and updating a chain-of-custody state associated with the one or more loaded xenon recovery modules through at least loaded, in-regeneration, certified, and returned-to-service states.

30. The method of claim 26, wherein delivering comprises modulating at least one of xenon concentration, flow rate, or delivery timing based on at least one user state selected from the group consisting of a physiological state, a neurological state, an electrophysiological state, a biometric state, and a behavioral state, and the method further comprises permitting the user to breathe at substantially ambient pressure throughout delivery without a tight face seal.
